SEQUENCE LISTING (119 sequences)

SEQ ID NO: 1 - protein, 20 aa, Artificial Sequence
  Signal Peptide
METDTLLVFVLLVWVPAGNG

SEQ ID NO: 2 - protein, 21 aa, Artificial Sequence
  Hinge1
GSSSSSSSSGSCDKTHTCPPC

SEQ ID NO: 3 - protein, 20 aa, Artificial Sequence
  Hinge2
GSSSSSSSGSCDKTHTCPPC

SEQ ID NO: 4 - protein, 19 aa, Artificial Sequence
  Hinge3
GSSSSSSGSCDKTHTCPPC

SEQ ID NO: 5 - protein, 18 aa, Artificial Sequence
  Hinge4
GSSSSSGSCDKTHTCPPC

SEQ ID NO: 6 - protein, 16 aa, Artificial Sequence
  Hinge5
GSSSGSCDKTHTCPPC

SEQ ID NO: 7 - protein, 20 aa, Artificial Sequence
  Hinge6
GSSSSSSSSGSDKTHTCPPC

SEQ ID NO: 8 - protein, 19 aa, Artificial Sequence
  Hinge7
GSSSSSSSGSDKTHTCPPC

SEQ ID NO: 9 - protein, 18 aa, Artificial Sequence
  Hinge8
GSSSSSSGSDKTHTCPPC

SEQ ID NO: 10 - protein, 17 aa, Artificial Sequence
  Hinge9
GSSSSSGSDKTHTCPPC

SEQ ID NO: 11 - protein, 15 aa, Artificial Sequence
  Hinge10
GSSSGSDKTHTCPPC

SEQ ID NO: 12 - protein, 13 aa, Artificial Sequence
  Hinge11
GSGSDKTHTCPPC

SEQ ID NO: 13 - protein, 13 aa, Artificial Sequence
  Linker-1
DKTHGSGSSSSSS

SEQ ID NO: 14 - protein, 11 aa, Artificial Sequence
  Linker-2
DKTHGSGSSSS

SEQ ID NO: 15 - protein, 8 aa, Artificial Sequence
  Linker3
DKTHGSGS

SEQ ID NO: 16 - protein, 9 aa, Artificial Sequence
  Linker-4
GSGSSSSSS

SEQ ID NO: 17 - protein, 6 aa, Artificial Sequence
  Linker-5
GSGSSS

SEQ ID NO: 18 - protein, 4 aa, Artificial Sequence
  Linker-6
GSGS

SEQ ID NO: 19 - protein, 8 aa, Artificial Sequence
  Linker-7
GGGSGGGS

SEQ ID NO: 20 - protein, 218 aa, Artificial Sequence
  Fc-N297S-knob
PAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 218 aa, Artificial Sequence
  Fc-N297S-hole
PAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 218 aa, Artificial Sequence
  Fc-Knob-US20160200833A1
PAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 218 aa, Artificial Sequence
  Fc-hole-US20160200833A1
PAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 24 - protein, 217 aa, Artificial Sequence
  Cambridge-Fc-knob
PAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 217 aa, Artificial Sequence
  Cambridge-Fc-hole
PAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 106 aa, Artificial Sequence
  IGG1-CL-kappa
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 103 aa, Artificial Sequence
  IGG1-CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 28 - protein, 330 aa, Artificial Sequence
  IGG1-CH1CH2sCH3-knob
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 330 aa, Artificial Sequence
  IGG1-CH1CH2sCH3-RF-knob
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNRFTQKSLSLSPGK

SEQ ID NO: 30 - protein, 330 aa, Artificial Sequence
  IGG1-CH1CH2sCH3-hole
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 31 - protein, 330 aa, Artificial Sequence
  IGG1-CH1CH2sCH3-RF-hole
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 32 - protein, 675 aa, Artificial Sequence
  scCD40L-Fc-knob_a
QIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 670 aa, Artificial Sequence
  scCD40L-Fc-knob_b
QIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 675 aa, Artificial Sequence
  scCD40L-Fc-hole_a
QIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 35 - protein, 670 aa, Artificial Sequence
  scCD40L-Fc-hole_b
QIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 681 aa, Artificial Sequence
  scCD27L-Fc-knob_a
QSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 676 aa, Artificial Sequence
  scCD27L-Fc-knob_b
QSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 681 aa, Artificial Sequence
  scCD27L-Fc-hole_a
QSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 39 - protein, 676 aa, Artificial Sequence
  scCD27L-Fc-hole_b
QSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 618 aa, Artificial Sequence
  scGITRL-Fc-knob_a
QPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 613 aa, Artificial Sequence
  scGITRL-Fc-knob_b
QPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 618 aa, Artificial Sequence
  scGITRL-Fc-hole_a
QPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 613 aa, Artificial Sequence
  scGITRL-Fc-hole_b
QPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 452 aa, Artificial Sequence
  aCD95L-HC-knob
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 452 aa, Artificial Sequence
  aCD95L-HC-hole
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 46 - protein, 452 aa, Artificial Sequence
  aCD95L-HC-RF-hole
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 47 - protein, 219 aa, Artificial Sequence
  aCD95L-LC
DIQMTQSPSSLSASVGDRVTITCKASQSIRTSLVWYQQKPGKAPKLLIYKASDLPSGVPSRFSGSGSGTDFTLTISSLQPEDFATYYCQSYDFRDTINNGHSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 451 aa, Artificial Sequence
  aCEA-HC-knob
EVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 451 aa, Artificial Sequence
  aCEA-HC-hole
EVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 50 - protein, 451 aa, Artificial Sequence
  aCEA-HC-RF-hole
EVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 51 - protein, 213 aa, Artificial Sequence
  aCEA-LC
QTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 450 aa, Artificial Sequence
  aPD-L1-HC-knob
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 450 aa, Artificial Sequence
  aPD-L1-HC-hole
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 54 - protein, 450 aa, Artificial Sequence
  aPD-L1-HC-RF-hole
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 55 - protein, 217 aa, Artificial Sequence
  aPD-L1-LC
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 676 aa, Artificial Sequence
  aCEA-hc-scCD40L-RBD
EVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSSASTKGPSVFPFAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 57 - protein, 677 aa, Artificial Sequence
  aCD95L-hc-scCD40L-RBD
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 58 - protein, 675 aa, Artificial Sequence
  aPDL1-hc-scCD40L-RBD
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 59 - protein, 121 aa, Artificial Sequence [sequence omitted]

SEQ ID NO: 60 - protein, 110 aa, Artificial Sequence
  aCD137-VL
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFGGGTKVEIKR

SEQ ID NO: 61 - protein, 116 aa, Artificial Sequence
  aMeso-VH
EVQLVESGGGLVQPGGSLRLSCAASGYTFTTYWMHWVRQAPGKGLEWVGYIRPSTGYTEYNQKFKDRFTISADTSKNTAYLQMNSLRAEDTAVYYCARSRWLLDYWGQGTLVTVSS

SEQ ID NO: 62 - protein, 113 aa, Artificial Sequence
  aMeso-VL
DIQMTQSPSSLSASVGDRVTITCKSSQSVLYSSNQKNYLAWFQQKPGKAPKLLIYWASTRESGVPSRFSGSGSGTDFTLTISSLQPEDFATYFCHQYLSSYTFGQGTKVEIKR

SEQ ID NO: 63 - protein, 116 aa, Artificial Sequence
  aCD25-VH
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTTLTVSS

SEQ ID NO: 64 - protein, 107 aa, Artificial Sequence
  aCD25-VL
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGSGTKVEVKR

SEQ ID NO: 65 - protein, 113 aa, Artificial Sequence [sequence omitted]

SEQ ID NO: 66 - protein, 108 aa, Artificial Sequence
  aPD1-a-VL
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKR

SEQ ID NO: 67 - protein, 120 aa, Artificial Sequence [sequence omitted]

SEQ ID NO: 68 - protein, 112 aa, Artificial Sequence
  aPD1-b-VL
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKR

SEQ ID NO: 69 - protein, 439 aa, Artificial Sequence
  scCD40L-RBD
QIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKL

SEQ ID NO: 70 - protein, 445 aa, Artificial Sequence
  scCD27L-RBD
QSLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 71 - protein, 382 aa, Artificial Sequence
  scGITRL-RBD
QPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 72 - protein, 472 aa, Artificial Sequence
  scCD137L- RBD
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRV

SEQ ID NO: 73 - protein, 462 aa, Artificial Sequence
  scLIGHT-RBD
EVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMV

SEQ ID NO: 74 - protein, 120 aa, Artificial Sequence
  aPD-L1-VH
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 75 - protein, 111 aa, Artificial Sequence
  aPD-L1-VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVEIKR

SEQ ID NO: 76 - protein, 15 aa, Artificial Sequence
  Streptag-II element-1
SSSSSSAWSHPQFEK

SEQ ID NO: 77 - protein, 16 aa, Artificial Sequence
  Streptag-II element-2
GSSSSSSAWSHPQFEK

SEQ ID NO: 78 - protein, 18 aa, Artificial Sequence
  Hinge17
GSGSSSGSCDKTHTCPPC

SEQ ID NO: 79 - protein, 17 aa, Artificial Sequence
  Hinge12
GSGSGGGSDKTHTCPPC

SEQ ID NO: 80 - protein, 15 aa, Artificial Sequence
  Hinge13
GSGSGGGSTHTCPPC

SEQ ID NO: 81 - protein, 11 aa, Artificial Sequence
  Hinge14
GSGSTHTCPPC

SEQ ID NO: 82 - protein, 11 aa, Artificial Sequence
  Hinge15
GSDKTHTCPPC

SEQ ID NO: 83 - protein, 17 aa, Artificial Sequence
  Hinge16
GSGSSSGSDKTHTCPPC

SEQ ID NO: 84 - protein, 674 aa, Artificial Sequence
  scCD40L-Fc-knob_c
QIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSGNGSQIAAHVISEASSKTTSVLQWAEKGYYTMSNNLVTLENGKQLTVKRQGLYYIYAQVTFCSNREASSQAPFIASLSLKSPGRFERILLRAANTHSSAKPCGQQSIHLGGVFELQPGASVFVNVTDPSQVSHGTGFTSFGLLKLGSGSSSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 708 aa, Artificial Sequence
  scCD137L-V1-Fc-knob_a
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 703 aa, Artificial Sequence
  scCD137L-V1-Fc-knob_b
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 708 aa, Artificial Sequence
  scCD137L-V1-Fc-hole_a
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 88 - protein, 641 aa, Artificial Sequence
  scCD137L-V1-Fc-hole_b
RVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSGNGSQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 89 - protein, 715 aa, Artificial Sequence
  scCD137L-V2-Fc-knob_a
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 710 aa, Artificial Sequence
  scCD137L-V2-Fc-knob_b
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 715 aa, Artificial Sequence
  scCD137L-V2-Fc-hole_a
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 92 - protein, 710 aa, Artificial Sequence
  scCD137L-V2-Fc-hole_b
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 93 - protein, 707 aa, Artificial Sequence
  scCD137L-V3-Fc-knob_a
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 702 aa, Artificial Sequence
  scCD137L-V3-Fc-knob_b
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 707 aa, Artificial Sequence
  scCD137L-V3-Fc-hole_a
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 96 - protein, 702 aa, Artificial Sequence
  scCD137L-V3-Fc-hole_b
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 97 - protein, 698 aa, Artificial Sequence
  scLIGHT-Fc-knob_a
EVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 693 aa, Artificial Sequence
  scLIGHT-Fc-knob_b
EVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 698 aa, Artificial Sequence
  scLIGHT-Fc-hole_a
EVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSSSGSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 100 - protein, 693 aa, Artificial Sequence
  scLIGHT-Fc-hole_b
EVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSGNGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGSGSDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 101 - protein, 683 aa, Artificial Sequence
  aCD95L-hc-scCD27L-RBD
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 102 - protein, 681 aa, Artificial Sequence
  aPDL1-hc-scCD27L-RBD
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRPGSGSGNGSESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 103 - protein, 620 aa, Artificial Sequence
  aCD95L-hc-scGITRL-RBD
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSQPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 104 - protein, 618 aa, Artificial Sequence
  aPDL1-hc-scGITRL-RBD
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHGSGSSSSSSQPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFISGSGSGNGSEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 105 - protein, 479 aa, Artificial Sequence
  scCD137L-V2-RBD
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPE

SEQ ID NO: 106 - protein, 471 aa, Artificial Sequence
  scCD137L-V3-RBD
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPE

SEQ ID NO: 107 - protein, 476 aa, Artificial Sequence
  scCD137L-V4-RBD
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGNGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 108 - protein, 468 aa, Artificial Sequence
  scCD137L-V5-RBD
QGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPGSGSGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTP

SEQ ID NO: 109 - protein, 218 aa, Artificial Sequence
  Fc-N297S
PAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 218 aa, Artificial Sequence
  FC-WT
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 330 aa, Artificial Sequence
  IGG1-CH1CH2CH3-RF
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 112 - protein, 330 aa, Artificial Sequence
  IGG1-CH1CH2sCH3-RF
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 113 - protein, 121 aa, Artificial Sequence
  aCEA-a-VH
EVQLLESGGGLVQPGGSLRLSCATSGFTFTDYYMNWVRQAPGKGLEWLGFIGNKANGYTTEYSASVKGRFTISRDKSKSTLYLQMNTLQAEDSAIYYCTRDRGLRFYFDYWGQGTLVTVSS

SEQ ID NO: 114 - protein, 107 aa, Artificial Sequence
  aCEA-a-VL
QTVLTQSPSSLSVSVGDRVTITCRASSSVTYIHWYQQKPGLAPKSLIYATSNLASGVPSRFSGSGSGTDYTFTISSLQPEDIATYYCQHWSSKPPTFGQGTKVEVKR

SEQ ID NO: 115 - protein, 121 aa, Artificial Sequence
  aCEA-b-VH
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 116 - protein, 116 aa, Artificial Sequence
  aCEA-b-VL
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 117 - protein, 122 aa, Artificial Sequence
  aCD95L-VH
EVQLVESGGGLVQPGGSLRLSCAASGFSFSDHYWMCWVRQAPGKGLEWVACIYTADSDSYYADSVKGRFTISKDSSKNTLYLQMNSLRAEDTAVYYCARNGAYAGGPYGDLWGQGTLVTVSS

SEQ ID NO: 118 - protein, 113 aa, Artificial Sequence
  aCD95L-VL
DIQMTQSPSSLSASVGDRVTITCKASQSIRTSLVWYQQKPGKAPKLLIYKASDLPSGVPSRFSGSGSGTDFTLTISSLQPEDFATYYCQSYDFRDTINNGHSFGQGTKVEIKR

SEQ ID NO: 119 - protein, 416 aa, Artificial Sequence
  scCD27L-V2-RBD
DVAELQLDHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVGSGSDVAELQLDHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVGSGSDVAELQLDHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWV

(19) United States
(12) Patent Application Publication
(10) Pub. No.: US 2023/0212260 A1
(43) Pub. Date: Jul. 6, 2023

(54) MULTI-SPECIFIC IMMUNE MODULATORS

(71) Applicant: Apogenix AG, Heidelberg (DE)

(72) Inventors: Meinolf Thiemann, Schriesheim (DE); Oliver Hill, Neckarsteinach (DE); Christian Gieffers, Heidelberg (DE); Katharina Billian-Frey, Heidelberg (DE)

(21) Appl. No.: 17/998,879

(22) PCT Filed: May 17, 2021
(86) PCT No.: PCT/EP2021/063005

§ 371 Date: Nov. 15, 2022

(30) Foreign Application Priority Data
May 15, 2020 (EP) 20175060.1

(51) Int. Cl. C07K 14/705; C07K 16/28

(52) U.S. Cl. CPC C07K 14/70575; C07K 16/2827; C07K 2317/31; C07K 2317/35; C07K 2317/55; C07K 2317/526; C07K 2319/30

(57) ABSTRACT

The invention relates to the field of immunology and immuno-oncology. More specifically, the invention relates to multi-specific and bi-specific cytokine and antibody derivatives capable of cell and/or tissue targeting to locally enhance the immune response and to reduce systemic toxicity.

### DESCRIPTION

The diverse functions of the immune system are orchestrated by a complex and delicately balanced interplay of stimulatory and inhibitory signals. Many key regulators of immune cell function belong to the so-called tumor necrosis factor superfamily (TNFSF) and their cognate receptors, the so-called TNF receptor superfamily. The TNFSF consists of 19 structurally related ligands, each binding to one or more of the 29 members of the TNF receptor superfamily.

TNFSF receptors are of great importance in the anti-tumor immune response and the regulation of inflammatory processes. They are expressed by a wide variety of immune cells including T cells and antigen-presenting cell populations, such as dendritic cells and macrophages, as well as by tumor cells themselves. This diverse expression pattern highlights the critical role that TNFSF receptors play in many parts of the body and in the various phases of the anti-tumor immune response.

WO 2010/010051 discloses trivalent protein moieties as singular single-chain fusion proteins. Said fusion proteins comprise three soluble, stalk depleted TNF superfamily (TNFSF) receptor binding domains connected by short (3-8) amino acids based linkers. They are substantially non-aggregating and well suited for therapeutic applications. Further trivalent single-chain TNFSF receptor binding domains with reduced immunogenicity and altered stability are disclosed in WO 2015/164588, WO 2016/177771, WO 2017/068183, WO 2017/068180, WO 2017/068185, WO 2017/072080 and WO 2017/068192 (contents of all aforementioned patent applications incorporated by reference herein in their entirety).

Despite already disclosed trivalent TNFSF protein moieties, a need remains for specific targeting constructs thereof. Such targeting constructs will allow for locally enhanced or locally enriched TNF receptor superfamily (TNFRSF) agonistic activity. Consequently, one objective of the present invention was providing multi- and bi-functional fusion proteins comprising at least two different trivalent TNFSF protein moieties or a trivalent TNFSF protein moiety and a specific antigen binding moiety useful for tissue or cell targeting or activity modulation.

The invention further relates to a nucleic acid molecule encoding a fusion protein as described herein and to a cell or a non-human organism transformed or transfected with a nucleic acid molecule as described herein.

The invention also relates to a pharmaceutical or diagnostic composition comprising as an active agent a multi-specific fusion protein, a nucleic acid molecule, or a cell as described herein.

The invention also relates to a multi-specific fusion protein, a nucleic acid molecule, or a cell as described herein for use in therapy, e.g., the use of a fusion protein, a nucleic acid molecule, or a cell as described herein for the preparation of a pharmaceutical composition in the prophylaxis and/or treatment of disorders caused by, associated with and/or accompanied by dysfunction of TNFSF cytokines, particularly proliferative disorders, such as tumors, e.g. solid or lymphatic tumors; infectious diseases; inflammatory diseases; metabolic diseases; autoimmune disorders, e.g. rheumatoid and/or arthritic diseases; degenerative diseases, e.g. neurodegenerative diseases such as multiple sclerosis; apoptosis-associated diseases or transplant rejections.

### DESCRIPTION OF THE FIGURES

FIG. 1 Schematic layout of bispecific Fab-Fc/scTNFSF-RBD-Fc heteromeric fusion proteins (so called single-arm-bispecific or SAB proteins). Hetero-dimerization of trivalent scTNFSF-RBD-Fc and the Fab-Fc is based on either by wild-type or specific CH3-domain variants of respective Fc- moieties.

FIG. 2 Schematic layout of bispecific, hexavalent scTNFSF ligands. Hetero-dimerization of two trivalent scTNFSF-RBD-Fc fusion proteins is based on the CH3 domain. This can be achieved either by wild-type or specific CH3-domain variants of the respective Fc- moieties.

FIG. 3 Schematic layout of bispecific, trivalent targeting constructs; construction based on direct or linker mediated fusion of the Fab heavy chain moiety to the trivalent scTNFSF-RBD

FIG. 4 Cellular in vitro activity of GITR agonists is shown with a GITR Luciferase reporter gene assay. aPDL1-scGITRL-SAB shows significant GITR agonism which is lower compared to scGITRL-Fc

FIG. 5 Cellular in vitro activity of GITR agonists is shown with a GITR Luciferase reporter gene assay. aPDL1-scGITRL-SAB shows significant GITR agonism which is lower compared to scGITRL-Fc and higher compared to aPDL1-scGITRL(trivalent)

FIG. 6 Cellular in vitro activity of GITR agonists is shown with a GITR Luciferase reporter gene assay. aPDL1-scGITRL-SAB shows significant GITR agonism which is higher compared to aPDL1-scGITRL(trivalent). Activities is clearly enhanced for both compounds by cross-linking with anti-human Fc (x-link)

FIG. 7 Cellular in vitro activity of PD-L1 inhibitors is shown with a PDL1 Luciferase reporter gene assay. aPDL1-scGITRL-SAB shows significant PDL1 inhibition which is in a similar range compared to aPDL1-mAb

FIG. 8 Cellular in vitro activity of PD-L1 inhibitors is shown with a PDL1 Luciferase reporter gene assay. aPDL1-scCD27L-SAB shows significant PDL1 inhibition which is in a similar range compared to aPDL1-mAb.

FIG. 9 Cellular in vitro activity of PDL1 inhibitors is shown with a PDL1 Luciferase reporter gene assay. aPDL1-scCD40L-SAB shows significant PDL1 inhibition which is in a similar range compared to aPDL1-mAb.

FIG. 10 Cellular in vitro activity of PDL1 inhibitors is shown with a PDL1 Luciferase reporter gene assay. aPDL1-scCD40L and aPDL1-scCD40L-Fc show significant PDL1 inhibition which are in a similar range compared to aPDL1-mAb

FIG. 11 Cellular in vitro activity of PDL1 inhibitors is shown with a PDL1 Luciferase reporter gene assay. aPDL1-scCD40L-Fc shows significant PDL1 inhibition which is in a similar range compared to aPDL1-mAb

FIG. 12 Cellular in vitro activity of PD-L1 inhibitors is shown with a PDL1 Luciferase reporter gene assay. aPDL1-scCD40L-SAB and aPDL1-scCD40L show significant PDL1 inhibition which are in a similar range compared to aPDL1-mAb.

FIG. 13 In vitro activity of CD95L inhibitors is shown with a Jurkat A3 cellular assay by assessing antagonism of CD95L-induced apoptosis. Apoptosis inhibition is paralleled by a decrease in Caspase 3/7 activity. aCD95L-scCD40L, aCD95L-mAb and aCD95L-scCD40L-Fc show significant apoptosis inhibition which are in a similar range. aCD95L-mAb and aCD95L-scCD40L-Fc are a bit more active than aCD95L-scCD40L.

FIG. 14 In vitro activity of CD95L inhibitors is shown with a Jurkat A3 cellular assay by assessing antagonism of CD95L-induced apoptosis. Apoptosis inhibition is paralleled by a decrease in Caspase 3/7 activity. aCD95L-scCD40L and aCD95L-mAb show significant apoptosis inhibition which are in a similar range. aCD95L-mAb is a bit more active than aCD95L-scCD40L.

FIG. 15 Cellular in vitro activity of CD27 agonists is shown with a CD27 Luciferase reporter gene assay. aPDL1-scCD27L-SAB shows slight CD27 agonism which is clearly enhanced by cross-linking with anti-human Fc (x-link). scCD27L-Fc activity is higher and can be further enhanced by cross-linking with anti-human Fc (x-link).

FIG. 16 Cellular in vitro activity of CD40 agonists is shown with a CD40 Luciferase reporter gene assay. Trimeric CD40L and trimeric CD40L (stab) [stab = stabilized] show a basal activity in this assay. aPDL1-scCD40L-SAB and aPDL1-scCD40L show a clearly higher CD40 agonism which indicates the contribution of PDL1-targeting for CD40 agonistic activity. This finding is confirmed by the lower activity of aCD95L-scCD40L (this compound targets CD95L instead of PD-L1).

FIG. 17 Cellular in vitro activity of CD40 agonists is shown with a CD40 Luciferase reporter gene assay. Trimeric CD40L shows a basal activity in this assay. In comparison, aCD95L-scCD40L shows a higher CD40 agonism.

FIG. 18 Cellular in vitro activity of CD40 agonists is shown with a CD40 Luciferase reporter gene assay. scCD40L-Fc, aPDL1-scCD40L-SAB and aPDL1-scCD40L-Fc show a clear CD40 agonism which can be clearly enhanced by cross-linking with anti-human Fc (x-link). The hexavalent CD40L-formats scCD40L-Fc and aPDL1-scCD40L-Fc show higher agonistic activity than the trivalent CD40L-format aPDL1-scCD40L-SAB.

FIG. 19 Cellular in vitro activity of CD40 agonists is shown with a CD40 Luciferase reporter gene assay. scCD40L-Fc, aPDL1-scCD40L and trimeric CD40L (stab) show a clear CD40 agonism which can be clearly enhanced by cross-linking with StrepMAB Immo (x-link). The hexavalent CD40L-format scCD40L-Fc shows higher agonistic activity than the trivalent CD40L-format aPDL1-scCD40L with both formats being clearly more active than trimeric CD40L (stab) [stab = stabilized]

FIG. 20 Cellular in vitro activity of CD40 agonists is shown with a CD40 Luciferase reporter gene assay. CD40 agonists at a constant concentration (as indicated) were incubated with increasing concentrations of aPDL1-mAb of up to 100 µg/ml. As expected, CD40 agonism of scCD40L-Fc is not affected by aPDL1-mAb competition since this molecule does not comprise a PDL1-targeting domain. In contrast, aPDL1-scCD40L-SAB and aPDL1-scCD40L show clearly reduced CD40 agonism with increasing concentrations of competing aPDL1-mAb. In conclusion, the PD-L1-targeting domains of aPDL1-scCD40L-SAB and aPDL1-scCD40L clearly contribute to their CD40 agonistic activity

FIG. 21 Cellular in vitro activity of CD40 agonists is shown with a CD40 Luciferase reporter gene assay. CD40 agonists at a constant concentration (as indicated) were incubated with increasing concentrations of aPDL1-mAb of up to 100 µg/ml. As expected, CD40 agonism of scCD40L-Fc is not affected by aPDL1-mAb competition since this molecule does not comprise a PDL1-targeting domain. In contrast, aPDL1-scCD40L-SAB shows a clearly reduced CD40 agonism with increasing concentrations of competing aPDL1-mAb. In conclusion, the PDL1-targeting domain of aPDL1-scCD40L-SAB clearly contributes to its CD40 agonistic activity

FIG. 22 ELISA demonstrating the binding of CEA targeting constructs to CEA. aCEA-Fab, aCEA-scCD40L and aCEA-scCD40L-Fc bind to human CEA in a dose-dependent manner. Binding of aCEA-Fab and aCEA-scCD40L-Fc is comparable and clearly stronger than that of aCEA-scCD40L.

FIG. 23 ELISA demonstrating the binding of CEA targeting constructs to CEA. aCEA-Fab and aCEA-scCD40L bind to human CEA in a dose-dependent manner. Binding of aCEA-Fab is clearly stronger than that of aCEA-scCD40L.

FIG. 24 Depicts exemplary scTNF RBD sequences well suited for multi-specific and bispecific TNF superfamily fusion protein assemblies of the first, second and third aspect of the invention.

FIG. 25 Schematic layout of bispecific, trivalent targeting constructs; construction based on direct or linker mediated fusion of one (A) or two (B) single-domain antibody moieties (VHH) to the trivalent scTNFSF-RBD .

FIG. 26 Schematic layout of bispecific VHH-Fc/scTNFSF-RBD-Fc heteromeric fusion proteins (so called Single arm bispecific (SAB) proteins, based on one (A) or two (B) single-domain antibody moieties (VHH)). Hetero-dimerization of trivalent scTNFSF-RBD-Fc and the VHH—Fc is based on either wild-type or specific CH3-domain variants of respective Fc— moieties.

FIG. 27 Schematic layout of bispecific scFv-Fc/scTNFSF-RBD-Fc heteromeric fusion proteins (so called Single arm bispecific (SAB) proteins, based on a scFv antibody fragment). Hetero-dimerization of trivalent scTNFSF-RBD-Fc and the scFv-Fc is based on either wild-type or specific CH3-domain variants of respective Fc- moieties.

FIG. 28 Schematic layout of bispecific, hexavalent targeting Fab-scTNFSF-RBD-Fc fusion proteins. Construction is based on direct fusion of a Fab domain to the trivalent scTNFSF-RBD-Fc and subsequent homodimerization of Fab-scTNFSF-RBD-Fc via the Fc domain e.g. aPDL1-scCD40L-Fc.

FIG. 29 Cellular in vitro activity of CD137 agonists is shown with a CD137 Luciferase reporter gene assay. Cellular cross-linking scCD137L-Fc (A) and Urelumab (B) with HT1080 cells shows a moderate increase in activity, whereas the non-targeting control aCD95L-scCD137L-SAB (C) shows no relevant increase in activity. D aPDL1-scCD137L-SAB shows slight CD137 agonism which is massively boosted by cross-linking with HT1080 cells (99% of the HT1080 cells do express PD-L1). Peak activities are approx. 16-fold higher than the activity observed for the hexavalent scCD137L-Fc (see A).

FIG. 30 Cellular in vitro activity of CD27 agonists is shown with a CD27 Luciferase reporter gene assay. A Cellular cross-linking of the non-targeting control aCD95L-scCD27L-SAB with MDA-MB231 cells shows no increase in activity. B aPDL1-scCD27L-SAB shows slight CD27 agonism which is massively boosted by cross-linking with MDA-MB231 cells (99% of the MDA-MB231 cells do express PD-L1). Peak activities are approx. 3-fold higher than the activity observed for the hexavalent scCD27L-Fc (see A).

FIG. 31 Naive human T cell activation assay. Staining intensity decreases with every cell division, i.e., undivided cells are most positive for the tag it violet stain. Naive Pan T cells were isolated from Human PBMCs. Day 0 stimulation with 1 µg/ml coated anti-CD3 (OKT3) and addition of medium or 100 ng/ml aPDL1-scCD137L-SAB or 100 ng/ml aCD95L-scCD137L-SAB. Day 5 flow cytometry. The bispecific molecules aPDL1-scCD137L-SAB and aCD95L-scCD137L-SAB lead to a similar proliferation of T cells in the presence of anti-CD3 stimulation (24.6 and 25.2 % proliferation vs. 16.0% proliferation for CD3 stimulation alone).

FIG. 32 Monocytes were isolated from buffy coats from healthy human donors employing standard kits (Stem Cell). Differentiation of monocytes was achieved by adding 50 ng/ml GM-CSF for 3 days followed by 50 ng/ml GM-CSF + 50 ng/ml IL-4 for further 3 days. Cells were then treated with 100 ng/ml of the indicated compounds for 24 h followed by flow cytometry assessing CD86 and CD83 expression. The bispecific molecule aPDL1-scCD40L-SAB combining trivalent scCD40L with the anti-PD-L1 antibody fragment is the most potent activator of dendritic cells with an activation level (CD86+ / CD83+) of 88.31%. Thus, combining both moieties in one molecule is far more effective than having these moieties added as two separate molecules, i.e. aPD-L1 antibody + CD40L(trimer) which show an activation level of only 39.48 %. aCD40 and CD40L(trimer) both show a moderate level of activity (40.59 % and 46.10 %), whereas aPD-L1 has the same activity as medium control.

FIG. 33 Monocytes were isolated from buffy coats from healthy human donors employing standard kits (Stem Cell). Differentiation of monocytes was achieved by adding 50 ng/ml GM-CSF for 3 days followed by 50 ng/ml GM-CSF + 50 ng/ml IL-4 for further 3 days. Cells were then treated with 100 ng/ml of the indicated compounds for 24 h followed by flow cytometry assessing CD86 and CD83 expression. The bispecific molecules combining trivalent scCD40L with the anti-PD-L1 antibody fragment are very potent activators of dendritic cells with an activation level (CD86+ / CD83+) similar to that of scCD40L-Fc: 73.00% for aPDL1-scCD40L-SAB, 87.18% for aPDL1-scCD40L(trivalent) and 96.12 % for aPDL1-scCD40L-Fc (format shown in FIG. 28). In contrast, the bispecific molecule aCD95L-scCD40L-SAB which targets CD95L instead of PD-L1 has only a moderate level of activation due to the lack of CD95L expression on monocytes. Low activation levels are also seen for aCD40 and CD40L(trimer).

FIG. 34 Cellular in vitro activity of GITR agonists is shown with a GITR Luciferase reporter gene assay. aPDL1-scGITRL shows significant GITR agonism which is lower compared to scGITRL-Fc. Activity of the trivalent GITR agonist aPDL1-scGITRL is clearly enhanced by cross-linking with anti-human Fc (x-link) to the level observed for the hexavalent GITR agonist scGITRL-Fc.

### DETAILED DESCRIPTION OF THE INVENTION

### Definitions

Dimer formation: As used herein, dimerization means, that a polypeptide chain upon folding is capable to form a stable structure with a second polypeptide chain upon folding and that a certain dimerization domain implemented into the polypeptide chains is enforcing this process. Dimer formation takes places between these specific domains present in each of the both polypeptides. Examples for dimerization domains are well known in the art. In natural human IgA-, IgD- and IgG antibodies, the CH3-domain is the driving force for the dimerization of the heavy-chains. In natural IgE or IgM antibodies the CH4-domain is the structural and functional equivalent to the IgG-CH3 domain enforcing their heavy-chain dimerization. The CH3-domain or their equivalents are selective only for themselves. This means, that any polypeptide comprising a functional CH3-domain either by nature or by engineering approaches is capable to form a dimer with a second polypeptide comprising a functional CH3-domain due to the CH3/CH3 dimer formation.

Hetero-dimerization of two CH3-domain comprising polypeptides to a functional bispecific fusion protein is achieved by co-expression of both polypeptides in a suitable host cell ensuring the presence of both chains simultaneously during protein folding. During the protein synthesis in the host-cell, any CH3-domain combination of the present polypeptide chains will be formed: heterodimers as well as homodimers. The wanted heterodimeric protein product needs to be purified afterwards by suitable chromatographic procedures. Methods for co-expression of CH3-comprising polypeptides and subsequent purification concepts for the heterodimeric product are well known in the art. The CH3-domains used can be either wild-type or they can comprise point mutations stabilizing a certain assembly e.g. as described by Carter et al. (Merchant, A., Zhu, Z., Yuan, J. et al. An efficient route to human bispecific IgG. Nat Biotechnol 16,677-681 (1998). https://doi.org/10.1038/nbt0798-677). For the generation of multi-specific immune modulators of the current invention, the usage of CH3-domain derived dimerization technologies is highly preferred. In a preferred embodiment, the CH3 domains implemented into both fusion protein polypeptides is a natural occurring sequence. In a preferred embodiment, the CH3 domains comprise point mutations, which are intended to stabilize the current dimerization product. It is highly preferred, that the stabilizing mutations result in covalent linkage of the both polypeptides, e.g. by cystines between the CH3-domains of a current assembly, thereby inhibiting the CH3-domain dissociation. As a consequence, interchain exchange reaction of the purified heterodimeric product and subsequent multimer and/or homodimer formation during the production are reduced. In a preferred embodiment, the CH3 domains comprise point mutations which preferentially lead to heterodimer formation during protein expression, e.g. knobs into hole (KiH) technology. In addition to the KiH technology, other more recent technologies to generate CH3 based heterodimerization domains have been developed employing either electrostatic steering or immunoglobulin domain interface exchange or a combination of both. The basic technologies present in the field are described in Skegro et al. J Biol Chem. 2017 Jun 9;292(23):9745-9759), Gunasekaran et al. J Biol Chem. 2010 Jun 18;285(25):19637-46, Sampei et al. PLoS One. 2013;8(2):e57479, Von Kreudenstein et al. MAbs. 2013 Sep-Oct;5(5):646-54, Davis et al. Protein Eng Des Sel. 2010 Apr;23(4):195-202.

Antibody: The terms “full length antibody”, “intact antibody”, “whole antibody” and “natural antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. As used herein, typical IgG derived constant heavy chain domains used in the context of the invention are SEQ-ID:28, SEQ-ID:29, SEQ-ID:30, SEQ-ID:31, SEQ-ID:111, SEQ-ID:112 all defined to start with Ala118 according to the EU numbering. As used herein a typical IgG derived CH1 domain used in the context of the invention is SEQ-ID:27, and a CLkappa is SEQ-ID: 26. The CH1 and CH2 domains are connected via a hinge region which stabilizes the antibody by cysteine bridges. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or µ (IgM), some of which may be further divided into subtypes, e.g. y1 (IgG1), y2 (IgG2), y3 (IgG3), y4 (IgG4), α1 (IgA1) and α2 (IgA2). The light chain of an antibody may be assigned to one of two types, called kappa (κ) and lambda (λ).In addition, hybrid light chain formats can be engineered comprising lambda VL and kappa CL, and vice versa. In a preferred embodiment, a light chain is based on a kappa LC or a hybrid LC composed of VLlambda/CLkappa for improved solubility and faster folding kinetics. As used herein a typical CL kappa domain used in the context of the invention is SEQ-ID: 26.

Antibody fragment: An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies, triabodies, tetrabodies, cross-Fab fragments; linear antibodies; single-chain antibody molecules (e.g. scFv); and single domain antibodies (e.g. VHH). For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). For a review on bispecific antibody fragment based constructs see, Brinkmann U, Kontermann RE. MAbs. 2017 Feb/Mar; 9(2):182-212. Single-domain antibodies are antibody fragments comprising all or a portion of the heavy chain variable domain or all or a portion of the light chain variable domain of an antibody. The first single domain antibodies were derived from the variable domain of the antibody heavy chain from camelids (nanobodies or VHH fragments). Furthermore, the term single domain antibody includes an autonomous human heavy chain variable domain (aVH) or VNAR fragments derived from sharks. In certain embodiments, a single domain antibody is a human single domain antibody (Domantis, Inc., Waltham, Mass.; see e.g. U.S. Pat. No. 6,248,516 B1). Methods for the preparation of antibody fragments are familiar to those skilled in the art. Widely used methods include proteolytic digestion or recombinant production in host cells. A non-limiting overview of methods of preparation of antibodies and antibody fragments is shown in US20160200833A1.

Fab-Fragment and scFv fragment: The term “Fab fragment” refers to an antibody fragment comprising a light chain fragment composed of a VL domain and a constant domain of a light chain (CL), and a VH domain and a first constant domain (CH1) of a heavy chain. The CH1 and CL domains can either contain wild-type sequences or point mutations for improved association (CH1: L128F, EU numbering). A “single-chain variable fragment (scFv)” is a fusion protein of the variable regions of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96).

TNF-SF: The term “TNF ligand family member” or “TNF family ligand” or “TNF superfamily” (TNF-SF) refers to a pro-inflammatory cytokine. Cytokines in general, and in particular the members of the TNF ligand superfamily, play a crucial role in the stimulation and coordination of the immune system. At present, nineteen cytokines have been identified as members of the TNF (tumor necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The TNF-SF ectodomain comprises the stalk region and the C-terminal located sequence known as TNF homology domain (THD), which has 20-30% amino acid identity between the superfamily members. The C-terminal part of the TNF ectodomain is also responsible for the TNF ligands to form trimeric complexes that are recognized by their specific receptors. These trimeric complexes are the binding competent structures as the receptor binding takes place at the protomer interfaces of the so called TNF-SF Receptor-binding-domain (RBD). In other words: the C-terminal regions of three individual TNF-SF polypeptides form a functional unit and trimer formation is a structural prerequisite for proper receptor recruitment of the human TNF-SF members.

Fc-Domain: The term “Fc domain” or “Fc region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. However, as often used herein, the Fc extends from amino acid residue P230 to amino acid K447 (CH2: 230-340, CH3: 341-447). The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The position N297 of the CH2 domain is glycosylated in a native sequence and required for Fc receptor binding. In one embodiment, a mutation at N297 abrogates Fc receptor binding. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote hetero-dimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Edelman, G.M. et al., Proc. Natl. Acad. USA, 63, 78-85 (1969).

The “knob-into-hole” technology is described e.g. in U.S. Pat. No. 5,731,168; U.S. Pat. No. 7,695,936. Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU numbering. As used herein, typical IgG derived Fc-domains used in the context of the invention are SEQ-ID:20, SEQ-ID:21, SEQ-ID:22, SEQ-ID:23, SEQ-ID:24, SEQ-ID:25, SEQ-ID:20, SEQ-ID:109 and SEQ-ID:110 all defined to start with Pro230 according to the EU numbering.

A “region equivalent to the Fc region of an immunoglobulin” is intended to include naturally occurring allelic variants of the Fc region of an immunoglobulin (e.g. D356E/L358M) as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

As used herein, the terms single-chain TNF-SF receptor binding domain, single-chain TNFSF receptor binding domain and TNF-SF RBD and TNFSF RBD are used synonymously for the above mentioned trivalent non-aggregating TNF-SF receptor binding domains. In addition, when referring to said receptor binding domains, the expression ‘single-chain’ is often abbreviated as ‘sc’, e.g. scTNFSF-RBD.

As used herein, anti PD-L1 antibodies or antibody fragments with anti-PD-L1 specificity are often referred to as “aPDL1” or “aPD-L1” antibodies or respective antibody fragments. The same is done for other antibody specificities; for example, for anti-CD95L, aCD95L is also used and for anti-CEA, aCEA is also used.

In the instant description, the protein assemblies of the first aspect of the invention are referred to as “single-arm bispecifics” or SABs.

In addition, especially when naming molecules or protein assemblies of the invention, the term antibody is often abbreviated to “AB” or “Ab”.

In addition, the terms “heteromeric fusion proteins” and “heteromeric protein assemblies” or “protein assemblies” are used interchangeably.

### EMBODIMENTS

According to the present invention, the multi-specific TNF superfamily fusion protein assemblies comprise at least (i) one protein moiety which comprises a single-chain TNF superfamily receptor binding domain and (ii) a protein moiety capable of specific binding to a cell surface antigen or an activity modulating effector.

In a first aspect of the invention, the bispecific TNF superfamily fusion protein assembly comprises at least
(a) a single-chain TNF-SF receptor binding domain fused to
(b) a first peptide linker fused to
(c) a first (hetero-)dimerization domain and
(d) an antigen binding or interacting protein moiety fused to
(e) a second peptide linker fused to
(f) a second (hetero-)dimerization domain

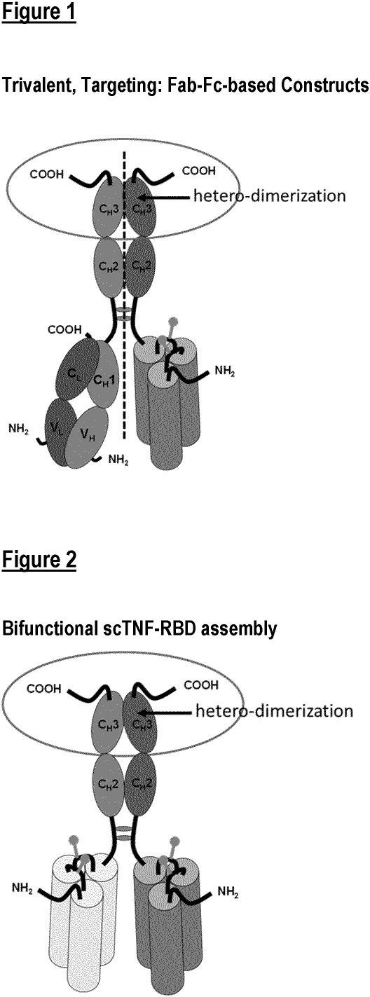

A general overview of a multi-specific TNF superfamily fusion protein assembly of the first aspect of the invention is given in FIG. 1.

As depicted in FIG. 1, a typical multispecific immune-modulator of the invention is a protein-unit comprising an IgG antibody-derived heavy and light chain assembly on one side and a trivalent single-chain TNFSF-RBD-Fc fusion polypeptide on the other side. The hetero-dimerization of both halves of the protein-unit is enforced by the CH3-domains and additionally stabilized by the hinge interchain cysteines. The co-expression and correct assembly of three polypeptide chains is necessary to form this functional bispecific protein unit. This fusion protein format is called Ab-scTNFSF-SAB (SAB=single-arm-bispecific).

In a preferred embodiment, the Ab-scTNFSF-SAB multispecific immune-modulator combines anti-PD-L1 (aPDL1) targeting with CD40 agonism. This specific assembly is called aPDL1-scCD40L-SAB. A non-limiting example comprises as mature protein the polypeptides SEQ-ID:33 (scCD40L-Fc-knob_b) or SEQ-ID:84 (scCD40L-Fc-knob_c) with SEQ-ID:55 (aPD-L1-LC) and SEQ-ID:54 (aPD-L1-HC-RF-hole).

In a preferred embodiment, the Ab-scTNFSF-SAB multispecific immune-modulator combines anti-PD-L1 (aPDL1) targeting with CD27 agonism. This specific assembly is called aPDL1-scCD27L-SAB. A non-limiting example comprises as mature protein the polypeptides SEQ-ID:39 (scCD27L-Fc-knob_b), SEQ-ID:55 (aPD-L1-LC) and SEQ-ID:54 (aPD-L1-HC-RF-hole).

A further preferred embodiment employs the TNFSF module SEQ-ID:119 (scCD27L-V2-RBD).

In a preferred embodiment, the Ab-scTNFSF-SAB multispecific immune-modulator combines anti-PD-L1 targeting with GITR agonism. This specific assembly is called aPDL1-scGITRL-SAB. A non-limiting example comprises as mature protein the polypeptides SEQ-ID:41 (scGITRL-Fc-knob_b), SEQ-ID:55 (aPD-L1-LC) and SEQ-ID:54 (aPD-L1-HC-RF-hole).

In a preferred embodiment, the Ab-scTNFSF-SAB multispecific immune-modulator combines anti-PD-L1 targeting with CD137 agonism. This specific assembly is called aPDL1-scCD137L-SAB. Non-limiting examples comprise as mature proteins the polypeptides SEQ-ID:86 (scCD137L-V1-Fc-knob_b) or SEQ-ID:90 (scCD137L-V2-Fc-knob_b) or SEQ-ID:94 (scCD137L-V3-Fc-knob_b) combined with SEQ-ID:55 (aPD-L1-LC) and SEQ-ID:54 (aPD-L1-HC-RF-hole). A further preferred embodiment employs the TNFSF module SEQ-ID:107 (scCD137L-V4-RBD) or SEQ-ID:108 (scCD137L-V5-RBD).

In a preferred embodiment, the Ab-scTNFSF-SAB multispecific immune-modulator combines anti-PD-L1 targeting with HVEM/LTbR- agonism. This specific assembly is called aPDL1-scLIGHT-SAB. A non-limiting example comprises as mature protein the polypeptides SEQ-ID:98 (scLIGHT-Fc-knob_b), SEQ-ID:55 (aPD-L1-LC) and SEQ-ID:54 (aPD-L1-HC-RF-hole).

In a preferred embodiment, the aforementioned PD-L1 specific Ab-scTNFSF-SAB (SAB=single-arm-bispecific) multispecific immune modulators (aPDL1-scCD40L-SAB, aPDL1-scCD27L-SAB, aPDL1-scGITRL-SAB, aPDL1-scCD137L-SAB, aPDL1-scLIGHT-SAB) comprise the same antigen-specific sequences (the VHCH and the VLCL of an aPD-L1 antibody) and the same trivalent scTNFSF-module but different CH3 domain sequences in their Fc part. The CH3 domain can be mutated or can be wild-type but still is capable to form a dimer with its counterpart leading the structural assembly as illustrated in FIG. 1. Non-limiting examples of IgG-derived CH3 domains are represented as part of SEQ-ID:44, SEQ-ID:45, SEQ-ID:48, SEQ-ID:49, SEQ-ID:52, SEQ-ID:53, SEQ-ID:28, SEQ-ID:29, SEQ-ID:30, SEQ-ID:31, SEQ-ID:111, SEQ-ID:112, SEQ-ID:20, SEQ-ID:21, SEQ-ID:22, SEQ-ID:23, SEQ-ID:24, SEQ-ID:25, SEQ-ID:109, SEQ-ID:110.

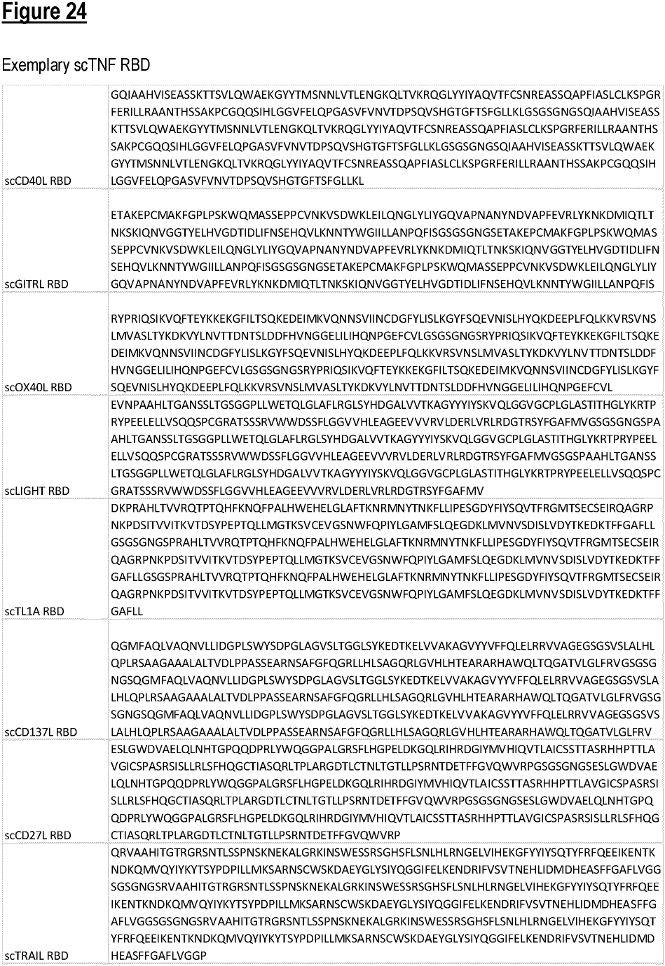

Using the above examples of aPDL1-specific, single-arm-bispecific immune modulators, it is obvious to the skilled person that this design can easily be combined with the scTNFSF RBDs as shown in FIG. 24, or variations thereof, to construct further aPDL1-SABs with CD40L, GITRL, OX40L, LIGHT, TL1A, CD137L, CD27L or TRAIL as the second specific binding target.

In a preferred embodiment, the Ab-scTNFSF-SAB multispecific immune-modulator combines anti-CD95L (aCD95L) targeting with CD40 agonism. This specific assembly is called aCD95L-scCD40L-SAB. A non-limiting example comprises as mature protein the polypeptides SEQ-ID:33 (scCD40L-Fc-knob_b), SEQ-ID:47 (aCD95L-LC) and SEQ-ID:46 (aCD95L-HC-RF-hole).

In a preferred embodiment, the Ab-scTNFSF-SAB multispecific immune-modulator combines anti-CD95L (aCD95L) targeting with CD27 agonism. This specific assembly is called aCD95L-scCD27L-SAB. A non-limiting example comprises as mature protein the polypeptides SEQ-ID:39 (scCD27L-Fc-knob_b), SEQ-ID:47 (aCD95L-LC) and SEQ-ID:46 (aCD95L-HC-RF-hole). A further preferred embodiment employs the TNFSF module SEQ-ID:119 (scCD27L-V2-RBD).

In a preferred embodiment, the Ab-scTNFSF-SAB multispecific immune-modulator combines anti-CD95L targeting with GITR agonism. This specific assembly is called aCD95L-scGITRL-SAB. A non-limiting example comprises as mature protein the polypeptides SEQ-ID:41 (scGITRL-Fc-knob_b), SEQ-ID:47 (aCD95L-LC) and SEQ-ID:46 (aCD95L-HC-RF-hole).

In a preferred embodiment, the Ab-scTNFSF-SAB multispecific immune-modulator combines anti-CD95L targeting with CD137 agonism. This specific assembly is called aCD95L-scCD137L-SAB. Non-limiting examples comprise as mature proteins the polypeptides SEQ-ID:86 (scCD137L-V1-Fc-knob_b) or SEQ-ID:90 (scCD137L-V2-Fc-knob_b) or SEQ-ID:94 (scCD137L-V3-Fc-knob_b) combined with SEQ-ID:47 (aCD95L-LC) and SEQ-ID:46 (aCD95L-HC-RF-hole). A further preferred embodiment employs the TNFSF module SEQ-ID:107 (scCD137L-V4-RBD) or SEQ-ID:108 (scCD137L-V5-RBD).

In a preferred embodiment, the Ab-scTNFSF-SAB multispecific immune-modulator combines anti-CD95L targeting with HVEM/LTbR- agonism. This specific assembly is called aCD95L-scLIGHT-SAB. A non-limiting example comprises as mature protein the polypeptides SEQ-ID:98 (scLIGHT-Fc-knob_b), SEQ-ID:47 (aCD95L-LC) and SEQ-ID:46 (aCD95L-HC-RF-hole).

In a preferred embodiment, the aforementioned CD95L specific Ab-scTNFSF-SAB (SAB=single-arm-bispecific) multispecific immune modulators (aCD95L-scCD40L-SAB, aCD95L-scCD27L-SAB, aCD95L-scGITRL-SAB, aCD95L-scCD137L-SAB, aCD95L-scLIGHT-SAB) comprise the same antigen-specific sequences (the VHCH and the VLCL of an aCD95L antibody) and the same trivalent scTNFSF-module but different CH3 domain sequences in their Fc part. The CH3 domain can be mutated or can be wild-type but still is capable to form a dimer with its counterpart leading the structural assembly as illustrated in FIG. 1. Non-limiting examples of IgG-derived CH3 domains are represented as part of SEQ-ID:44, SEQ-ID:45, SEQ-ID:48, SEQ-ID:49, SEQ-ID:52, SEQ-ID:53, SEQ-ID:28, SEQ-ID:29, SEQ-ID:30, SEQ-ID:31, SEQ-ID:111, SEQ-ID:112, SEQ-ID:20, SEQ-ID:21, SEQ-ID:22, SEQ-ID:23, SEQ-ID:24, SEQ-ID:25, SEQ-ID:109, SEQ-ID:110.

Using the above examples of aCD95L-specific, single-arm-bispecific immune modulators, it is obvious to the skilled person that this design can easily be combined with the scTNFSF RBDs as shown in FIG. 24, or variations thereof, to construct further aCD95L-SABs with CD40L, GITRL, OX40L, LIGHT, TL1A, CD137L, CD27L or TRAIL as the second specific binding target.

In a preferred embodiment, the Ab-scTNFSF-SAB multispecific immune-modulator combines anti-CEA (aCEA) targeting with CD40 agonism. This specific assembly is called aCEA-scCD40L-SAB. A non-limiting example comprises as mature protein the polypeptides SEQ-ID:33 (scCD40L-Fc-knob_b), SEQ-ID:51 (aCEA-LC) and SEQ-ID:50 (aCEA-HC-RF-hole).

In a preferred embodiment, the Ab-scTNFSF-SAB multispecific immune-modulator combines anti-CEA (aCEA) targeting with CD27 agonism. This specific assembly is called aCEA-scCD27L-SAB. A non-limiting example comprises as mature protein the polypeptides SEQ-ID:39 (scCD27L-Fc-knob_b), SEQ-ID:51 (aCEA-LC) and SEQ-ID:50 (aCEA-HC-RF-hole). A further preferred embodiment employs the TNFSF module SEQ-ID:119 (scCD27L-V2-RBD).

In a preferred embodiment, the Ab-scTNFSF-SAB multispecific immune-modulator combines anti-CEA targeting with GITR agonism. This specific assembly is called aCEA-scGITRL-SAB. A non-limiting example comprises as mature protein the polypeptides SEQ-ID:41 (scGITRL-Fc-knob_b), SEQ-ID:51 (aCEA-LC) and SEQ-ID:50 (aCEA-HC-RF-hole).

In a preferred embodiment, the Ab-scTNFSF-SAB multispecific immune-modulator combines anti-CEA targeting with CD137 agonism. This specific assembly is called aCEA-scCD137L-SAB. Non-limiting examples comprise as mature proteins the polypeptides SEQ-ID:86 (scCD137L-V1-Fc-knob_b) or SEQ-ID:90 (scCD137L-V2-Fc-knob_b) or SEQ-ID:94 (scCD137L-V3-Fc-knob_b) combined with SEQ-ID:51 (aCEA-LC) and SEQ-ID:50 (aCEA-HC-RF-hole). A further preferred embodiment employs the TNFSF module SEQ-ID:107 (scCD137L-V4-RBD) or SEQ-ID:108 (scCD137L-V5-RBD).

In a preferred embodiment, the Ab-scTNFSF-SAB multispecific immune-modulator combines anti-CEA targeting with HVEM/LTbR- agonism. This specific assembly is called aCEA-scLIGHT-SAB. A non-limiting example comprises as mature protein the polypeptides SEQ-ID:98 (scLIGHT-Fc-knob_b), SEQ-ID:51 (aCEA-LC) and SEQ-ID:50 (aCEA-HC-RF-hole).

In a preferred embodiment, the aforementioned CEA specific Ab-scTNFSF-SAB (SAB=single-arm-bispecific) multispecific immune modulators (aCEA-scCD40L-SAB, aCEA-scCD27L-SAB, aCEA-scGITRL-SAB, aCEA-scCD137L-SAB, aCEA-scLIGHT-SAB) comprise the same antigen-specific sequences (the VHCH and the VLCL of an aCEA antibody) and the same trivalent scTNFSF-module but different CH3 domain sequences in their Fc part. The CH3 domain can be mutated or can be wild-type but still is capable to form a dimer with its counterpart leading the structural assembly as illustrated in FIG. 1. Non-limiting examples of IgG-derived CH3 domains are represented as part of SEQ-ID:44, SEQ-ID:45, SEQ-ID:48, SEQ-ID:49, SEQ-ID:52, SEQ-ID:53, SEQ-ID:28, SEQ-ID:29, SEQ-ID:30, SEQ-ID:31, SEQ-ID:111, SEQ-ID:112, SEQ-ID:20, SEQ-ID:21, SEQ-ID:22, SEQ-ID:23, SEQ-ID:24, SEQ-ID:25, SEQ-ID:109, SEQ-ID:110.

Using the above examples of CEA-specific, single-arm-bispecific immune modulators, it is obvious to the skilled person that this design can easily be combined with the scTNFSF RBDs as shown in FIG. 24, or variations thereof, to construct further aCEA-SABs with CD40L, GITRL, OX40L, LIGHT, TL1A, CD137L CD27L, or TRAIL as the second specific binding target.

One specific variation of the scCD27L RBD comprises an exchange of the N-terminal glutamine of Seq-ID:36 (scCD27L-Fc-knob_a), Seq-ID:37 (scCD27L-Fc-knob_b), Seq-ID:38 (scCD27L-Fc-hole_a), Seq-ID:39 (scCD27L-Fc-hole_b), Seq-ID:70 (scCD27L-RBD) to glutamate.

In further preferred embodiments, the examples of Ab-scTNFSF-SAB, described in the first aspect of the invention, can be modified by persons skilled in the art by the exchange of the VH and VL domains from aPD-L1 to other antibody specificities, including but not limited to
anti-CD137 (SEQ-ID:59 aCD137-VH, SEQ-ID:60 aCD137-VL)
anti-Mesothelin (SEQ-ID:61 aMeso-VH, SEQ-ID:62 aMeso-VL)
anti-CD25 (SEQ-ID:63 aCD25-VH, SEQ-ID:64 aCD25-VL)
anti-PD-1 (SEQ-ID:65 aPD1-a-VH, SEQ-ID:66 aPD1-a-VL, SEQ-ID:67 aPD1-b-VH, SEQ-ID:68 aPD1-b-VL)
anti-CEA (SEQ-ID:113 aCEA-a-VH, SEQ-ID:114 aCEA-a-VL, SEQ-ID:115 aCEA-b-VH, SEQ-ID:116 aCEA-b-VL)
anti-CD95L (SEQ-ID:117 aCD95L-VH, SEQ-ID:118 aCD95L-VL), thus resulting in aCD137-scTNFSF-SAB, aMeso-scTNFSF-SAB, aCD25-scTNFSF-SAB, aPD1-scTNFSF-SAB, aCEA-scTNFSF-SAB or aCD95L-scTNFSF-SAB.

In the second aspect of the invention, the multi-specific TNF superfamily fusion protein assembly comprises at least
(a) a single-chain TNF-SF receptor binding domain fused to
(b) a first peptide linker fused to
(c) a first (hetero-)dimerization domain and
(d) a second single-chain TNF-SF receptor binding domain fused to
(e) a second peptide linker fused to
(f) a second (hetero-)dimerization domain

A general overview of a multi-specific TNF superfamily fusion protein assembly of the second aspect of the invention is given in FIG. 2.

As depicted in FIG. 2, a typical multispecific immune-modulator of the invention can be achieved by combining two scTNFSF-Fc fusion polypeptides of the invention. In a preferred embodiment, the multispecific immune-modulator comprises as the mature protein the polypeptides SEQ-ID:32 and SEQ-ID:36. In a preferred embodiment, the multispecific immune-modulator comprises as the mature protein the polypeptides SEQ-ID:33 and SEQ-ID:37. Both structures are bispecific for CD40 and CD27 with three binding sites for each of the both TNFRSF-members.

In the second aspect of the invention, the multi-specific TNF superfamily fusion protein assembly comprises at least a single-chain TNF-SF receptor binding in domain of part a) (columns) and one single-chain domain of part e) (rows). This allows for free combination of all disclosed single-chain TNF-SF receptor binding domains within the protein assembly. As a non-limited list, possible combinations of part a) (columns) and part e) (rows) are marked by an ‘X’ in the subsequent table.

| | | | | | | | | | | |
|---|---|---|---|---|---|---|---|---|---|---|
| | CD95 L | LIGHT | TRAIL | CD40 L | CD13 7L | CD27 L | OX40 L | GITRL | TL1A | TWEA K |
| CD95 L | | X | X | X | X | X | X | X | X | X |
| LIGHT | X | | X | X | X | X | X | X | X | X |
| TRAIL | X | X | | X | X | X | X | X | X | X |
| CD40 L | X | X | X | | X | X | X | X | X | X |
| CD13 7L | X | X | X | X | | X | X | X | X | X |
| CD27 L | X | X | X | X | X | | X | X | X | X |
| OX40 L | X | X | X | X | X | X | | X | X | X |
| GITRL | X | X | X | X | X | X | X | | X | X |
| TL1A | X | X | X | X | X | X | X | X | | X |
| TWEA K | X | X | X | X | X | X | X | X | X | |

In a third aspect of the invention, the multi-specific TNF superfamily fusion protein assembly comprises at least
(a) a functional Fab domain of an antibody fused to
(b) a single-chain TNF-SF receptor binding domain, wherein the C-terminal end of the constant heavy chain domain of the Fab fragment (a) is fused to the single-chain TNF-SF receptor binding via a peptide linker (Seq-ID:13-Seq-ID:19).

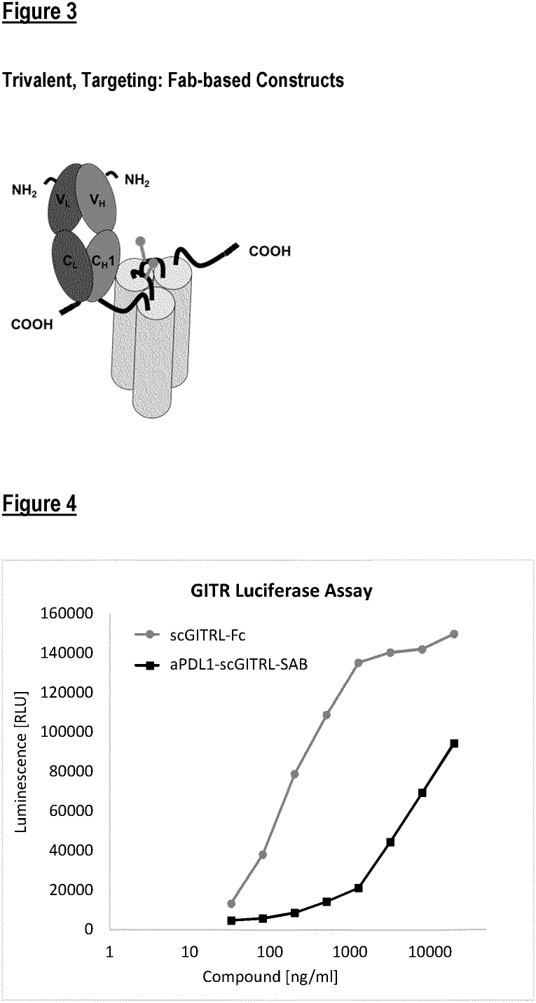

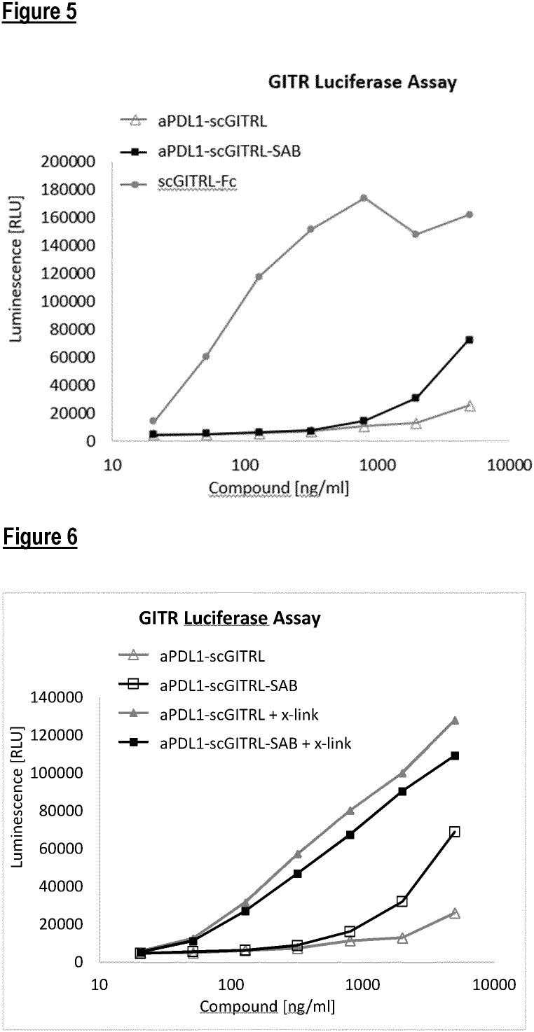

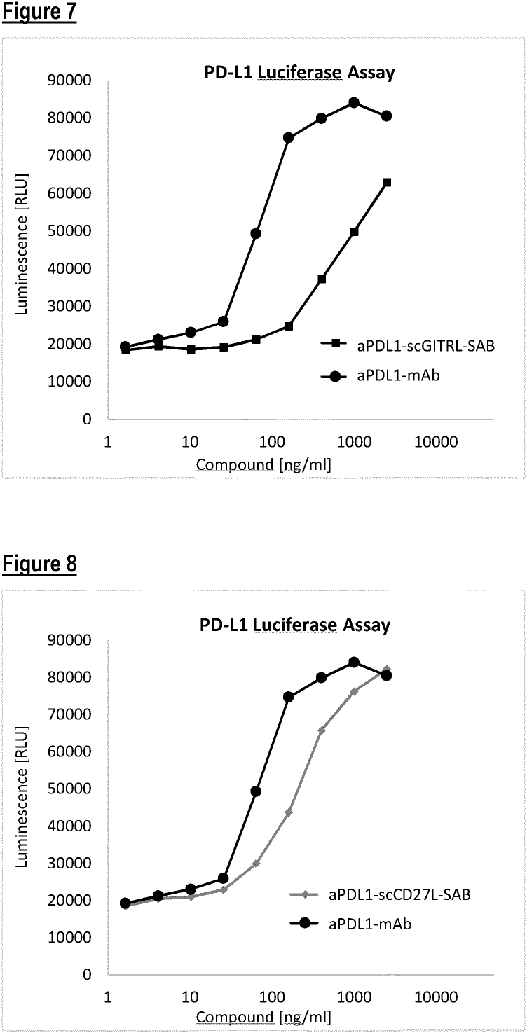

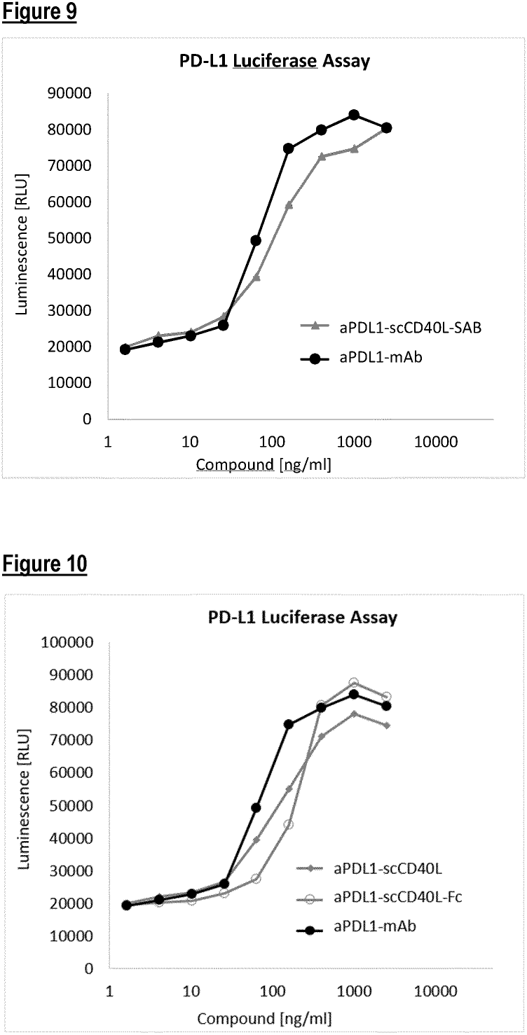

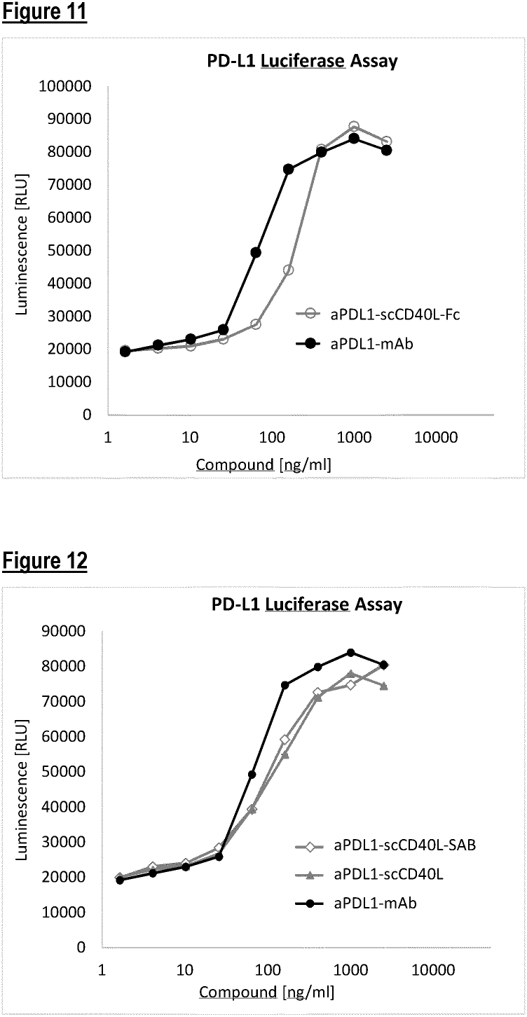

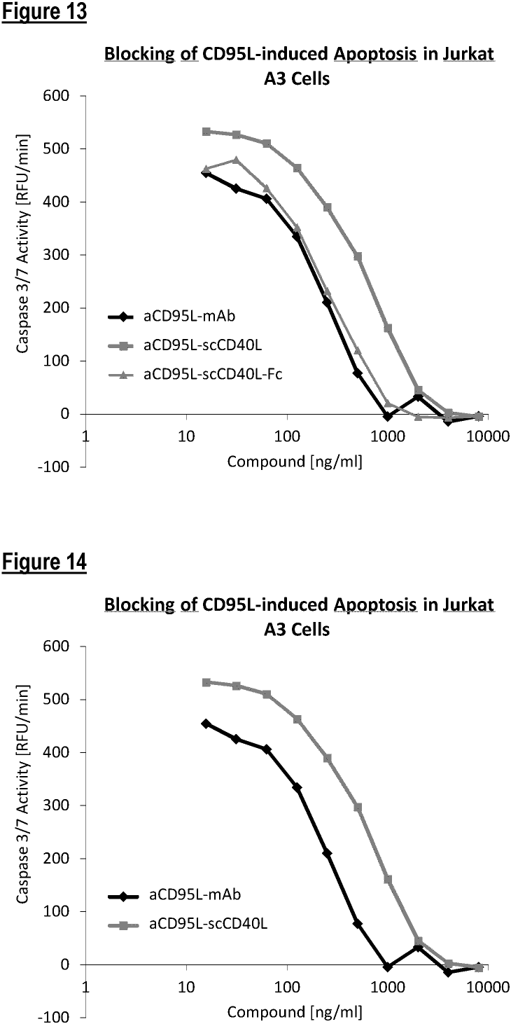

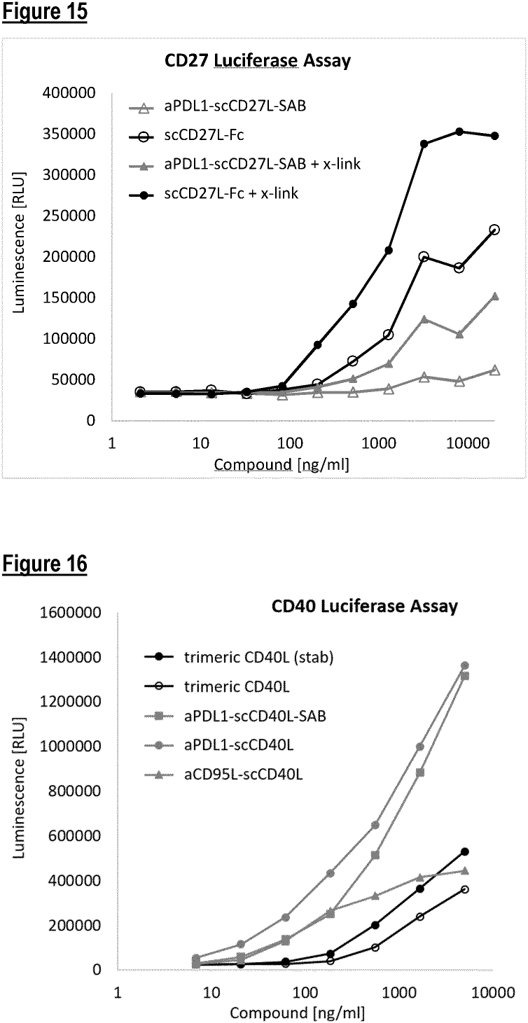

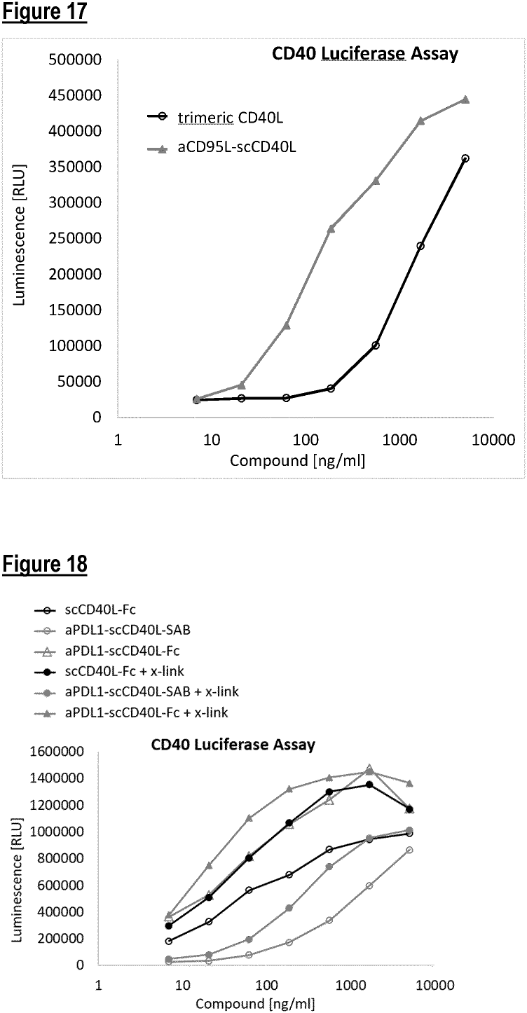

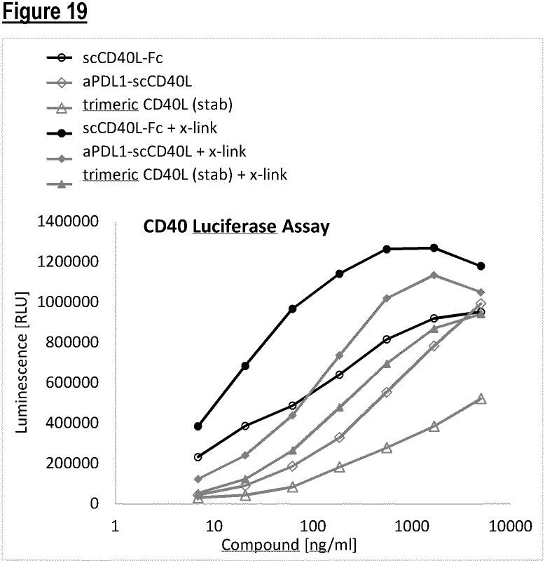

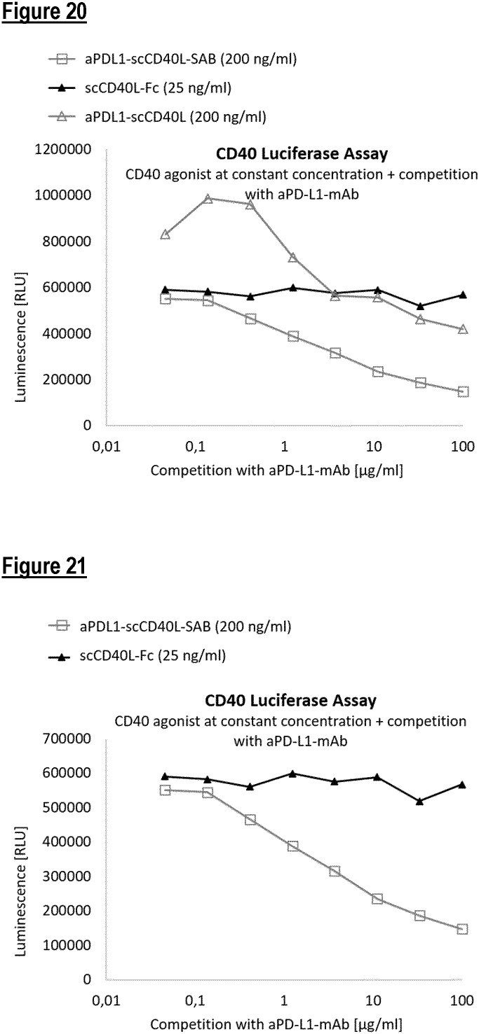

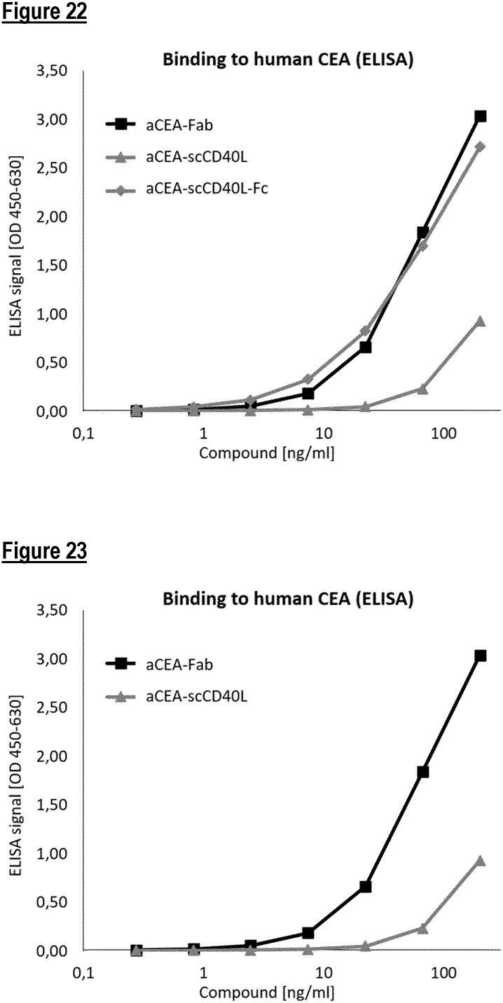

A general overview of a multi-specific TNF superfamily fusion protein assembly of the third aspect of the invention is given in FIG. 3. This fusion protein format is called Ab-scTNFSF. In a preferred embodiment, the Ab-scTNFSF multispecific immune-modulator combines anti-PD-L1 (aPDL1) targeting with CD40 agonism. This specific assembly is called aPDL1-scCD40L. A non-limiting example comprises as mature protein the polypeptides SEQ-ID:58 (aPDL1-hc-scCD40L-RBD) and SEQ-ID:55 (aPD-L1-LC).

In a preferred embodiment, the Ab-scTNFSF multispecific immune-modulator combines anti-PD-L1 (aPDL1) targeting with CD27 agonism. This specific assembly is called aPDL1-scCD27L. A non-limiting example comprises as mature protein the polypeptides SEQ-ID:102 (aPDL1-hc-scCD27L-RBD) and SEQ-ID:55 (aPD-L1-LC). A further preferred embodiment employs the TNFSF module SEQ-ID:119 (scCD27L-V2-RBD).

In a preferred embodiment, the Ab-scTNFSF multispecific immune-modulator combines anti-PD-L1 targeting with GITR agonism. This specific assembly is called aPDL1-scGITRL. A non-limiting example comprises as mature protein the polypeptides SEQ-ID:104 (aPDL1-hc-scGITRL-RBD) and SEQ-ID:55 (aPD-L1-LC).

In a preferred embodiment, the Ab-scTNFSF multispecific immune-modulator combines anti-CD95L (aCD95L) targeting with CD40 agonism. This specific assembly is called aCD95L-scCD40L. A non-limiting example comprises as mature protein the polypeptides SEQ-ID:57 (aCD95L-hc-scCD40L-RBD) and SEQ-ID:47 (aCD95L-LC).

In a preferred embodiment, the Ab-scTNFSF multispecific immune-modulator combines anti-CD95L (aCD95L) targeting with CD27 agonism. This specific assembly is called aCD95L-scCD27L. A non-limiting example comprises as mature protein the polypeptides SEQ-ID:101 (aCD95L-hc-scCD27L-RBD) and SEQ-ID:47 (aCD95L-LC). A further preferred embodiment employs the TNFSF module SEQ-ID:119 (scCD27L-V2-RBD).

In a preferred embodiment, the Ab-scTNFSF multispecific immune-modulator combines anti-CD95L targeting with GITR agonism. This specific assembly is called aCD95L-scGITRL. A non-limiting example comprises as mature protein the polypeptides SEQ-ID:103 (aCD95L-hc-scGITRL-RBD) and SEQ-ID:47 (aCD95L-LC).

In further preferred embodiments, the Ab-scTNFSF multispecific immune-modulator combines anti-CD95L targeting with CD137 agonism or with HVEM/LTbR- agonism. The specific assemblies are called aCD95L-scCD137L and aCD95L-scLIGHT. Persons skilled in the art can easily exchange the TNFSF module in the aforementioned Ab-scTNFSF examples for aPDL1 or aCD95L targeting by SEQ-ID:72 (scCD137L- RBD), SEQ-ID:105 (scCD137L-V2-RBD), SEQ-ID:106 (scCD137L-V3-RBD), SEQ-ID:107 (scCD137L-V4-RBD), SEQ-ID:108 (scCD137L-V5-RBD) or SEQ-ID:73 (scLIGHT-RBD).

In further preferred embodiments, the examples of Ab-scTNFSF, described in the third aspect of the invention, can easily be modified by persons skilled in the art by the exchange of the VH and VL domains from aPD-L1 or aCD95L to other antibody specificities, including but not limited to
anti-CD137 (SEQ-ID:59 aCD137-VH, SEQ-ID:60 aCD137-VL)
anti-Mesothelin (SEQ-ID:61 aMeso-VH, SEQ-ID:62 aMeso-VL)
anti-CD25 (SEQ-ID:63 aCD25-VH, SEQ-ID:64 aCD25-VL)
anti-PD-1 (SEQ-ID:65 aPD1-a-VH, SEQ-ID:66 aPD1-a-VL, SEQ-ID:67 aPD1-b-VH, SEQ-ID:68 aPD1-b-VL)
anti-CEA (SEQ-ID:113 aCEA-a-VH, SEQ-ID:114 aCEA-a-VL, SEQ-ID:115 aCEA-b-VH, SEQ-ID:116 aCEA-b-VL; SEQ-ID:56 aCEA-hc-scCD40L-RBD / SEQ-ID:51 aCEA-LC)
thus resulting in aCD137-scTNFSF, aMeso-scTNFSF, aCD25-scTNFSF, aPD1-scTNFSF or aCEA-scTNFSF.

Using the above examples of aCD95L-scTNFSF bispecific immune modulators, it is obvious to the skilled person that this design can easily be combined with the scTNFSF RBDs as shown in FIG. 24, or variations thereof, to construct further aCD95L-scTNFSF bispecific immune modulators with CD40L, GITRL, OX40L, LIGHT, TL1A, CD137L, CD27L, or TRAIL as the second specific binding target.

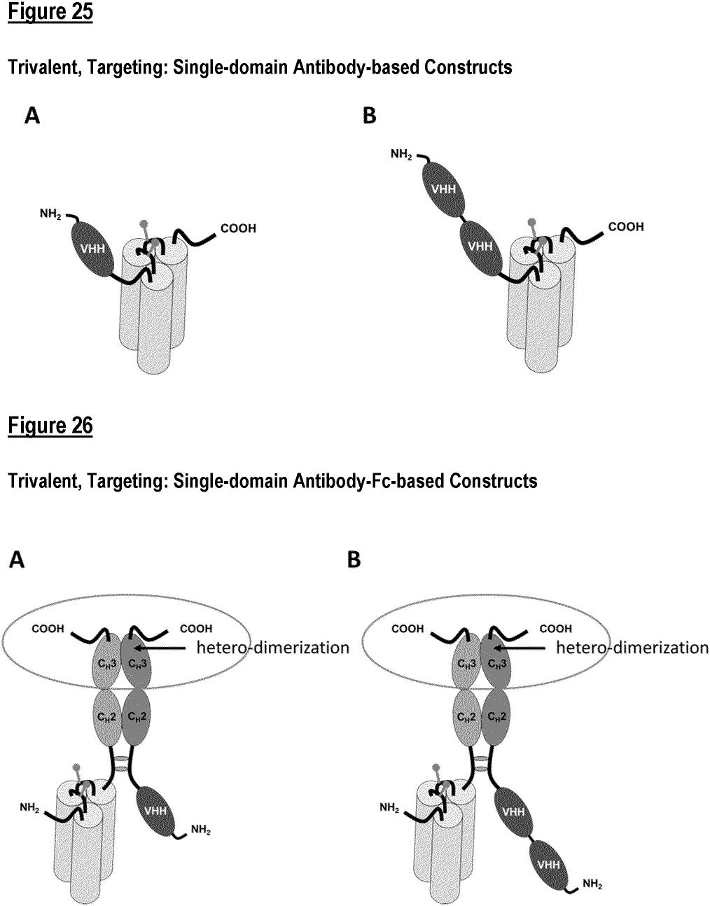

In a further aspect of the invention, the multi-specific TNF superfamily fusion protein assembly comprises at least
(a) a functional single VH (variable heavy chain) domain of an antibody fused to
(b) a single-chain TNF-SF receptor binding domain, wherein the c-terminal end of the VH domain is fused to the single-chain TNF-SF receptor binding via a peptide linker (FIG. 25).

Examples for functional single VH domains are the so called VH derived single domain antibodies (VHH).

From WO 2010/010051, the skilled person knows of methods for the construction of single-chain TNF-SF receptor binding domains suitable for use in any of the above-mentioned aspects of the invention. In general, suitable, non-aggregating TNF-SF receptor binding domains are made up of three soluble, stalk depleted receptor binding domains which are linked by short, preferable 3-8 amino acid long linkers.

In a special embodiment, the receptor binding domains can be linked by shorter linkers or even fused without additional amino acids.

As mentioned above, especially suited trivalent, non-aggregating TNF-SF receptor binding domains are disclosed in WO 2015/164588, WO 2016/177771, WO 2017/068183, WO 2017/068180, WO 2017/068185, WO 2017/072080 and WO 2017/068192. As a non-limiting example favorable single-chain TNF-SF receptor binding domains can be selected from sequences of FIG. 24.

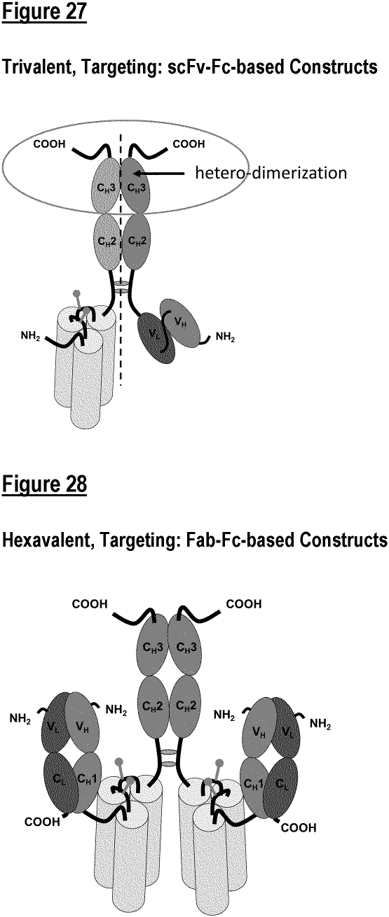

The antigen binding or interacting moiety of the first and/or third aspect of the invention can be an antibody fragment, for example a monospecific antibody fragment or a functional fragment thereof. Further suitable binding and interacting moieties are known in the art. Non-limiting examples are: single chain antibodies or functional fragments thereof, single domain antibodies, functional scFv fragments. Examples of these formats are shown in FIGS. 1, 26, 27 and FIGS. 3 and 25.

In a specific embodiment of the first and/or third aspect of the invention, the functional antibody fragment is directed against a cell surface marker or an activity-modulating target. As a non-limiting example the antibody or antibody fragment is directed against: tyrosine- kinase-receptors (EGFR, HER2, HER3, HER4), VEGFRs, heteromeric integrin a- or β-receptor family, including VLA-4 and LFA-1, E-selectin, L-selectin, P-selectin, tumor stroma markers like fibroblast activation protein (FAP), endoglyx-1, MCSP or endosialin, galectin, N-CAM (Myelin protein zero), ICAM1 - ICAM5, VCAM-1, PE-CAM, L1-CAM, Nectin (PVRL1, PVRL2, PVRL3), EpCAM, tumor antigens, including NY-ESO-1, MAGE1, MAGE2, CA-125, Carcinoembryonic Antigen (CEA), CAMPATH-1 (CD52), CD44 and tumor specific variants thereof and other tumor selective cell surface markers, CD2, CD5, CD7, CD19, CD20, CD21, CD22, CD24, CD25, CD30, CD33, CD38, CD40, CD52, CD56, CD71, CD72, CD73, CD105, CD117, CD123, CD133, c-Met, PDGFR, IGF1-R, HMW-MAA, TAG-72, GD2, GD3, GM2, folate receptor, Lgr5, Ley, Muc-1, Muc-2, PSMA, PSCA and uPAR. More preferably, the target molecule is FAP, EGFR, HER2 or HER, melanoma-associated chondroitin sulfate proteoglycan (MCSP).

The antibody or antibody fragment might also be directed against a member of the B7 family, including B7-1 (CD80), B7-2 (CD86), B7-DC (PDCD1LG2, PD-L2, CD273), B7-H1 (PD-L1, CD274), B7-H2 (ICOSLG, B7RP1, CD275), B7-H3 (CD276), B7-H4 (VTCN1), B7-H5 (VISTA, Platelet receptor Gi24, SISP1), B7-H6 (NCR3LG1) and B7-H7 (HHLA2).

In a further embodiment, the antibody or antibody fragment might also be directed against activity modulating targets, including but not limited to CTLA-4, PD1, CD3, CD4, CD8, CD28, HLA Class I and Class II, LAG3 (CD223), ICOS (CD278), CD39, CD73, TIGIT, CD96, PTA1 (CD226), TIM-3, TIM-1, CD47, SIRP-alpha, DNAM-1, and Interleukins (anti-inflammatory), including but not limited to IL4, IL6, IL9, IL10, IL11, IL13, IL18, IL21 and IL22.

It has to be noted that all ectodomains of the TNF-SF and TNFR-SF are especially suited targets for antibody fragments of the first aspect of the invention. A preferred but not-limiting list comprises ectodomains of TNF-SF ligand domains like CD95L, TNF-alpha, CD40L, CD27L, LIGHT, TL1A and TWEAK and TNF-receptor domains like CD40, CD27, 4-1BB, OX40, GITR, HVEM, BCMA, LTBR and TWEAKR.

Examples of antibodies binding to the ectodomains of the TNFR-SF are the anti-CD137 mAbs Urelumab and Utomilumab. Further examples of monoclonal antibodies binding to the ectodomains of the TNFR-SF are Varlilumab (anti-CD27), Selicrelumab (anti-CD40), APX005M (anti-CD40) and TRX518 (anti-GITR).

From a scientific and commercial point of view, combinations of TNFSF ligands with antibodies that bind already evaluated surface markers of cancer cells, such as CEA or HER2, or that intervene in the signaling cascade of checkpoint modulators (PD-1, CTLA4, CD95) are particularly attractive. The peptides with anti-PDL1 (aPDL1) and anti-CD95L (aCD95L) or anti-CEA (aCEA) activity shown in the examples and figures represent therefore further particularly preferred embodiments of the invention.

A further aspect of the present invention relates to nucleic acid molecules encoding protein moieties of multi-specific fusion proteins as described herein. The nucleic acid molecule may be a DNA molecule, e.g. a double-stranded or single-stranded DNA molecule, or an RNA molecule. The nucleic acid molecule may encode the fusion protein or a precursor thereof, e.g. a pro- or pre-proform of the fusion protein which may comprise a signal sequence or other heterologous amino acid portions for secretion or purification which are preferably located at the N- and/or C-terminus of the fusion protein. The heterologous amino acid portions may be linked to the first and/or second domain via a protease cleavage site, e.g. a Factor Xₐ, thrombin or IgA protease cleavage site.

The nucleic acid molecule may be operatively linked to an expression control sequence, e.g. an expression control sequence that allows expression of the nucleic acid molecule in a desired host cell. The nucleic acid molecule may be located on a vector, e.g. a plasmid, a bacteriophage, a viral vector, a chromosomal integration vector, etc. Examples of suitable expression control sequences and vectors are described for example by Sambrook et al. (1989) Molecular Cloning, A Laboratory Manual, Cold Spring Harbor Press, and Ausubel et al. (1989), Current Protocols in Molecular Biology, John Wiley & Sons or more recent editions thereof.

Various expression vector/host cell systems may be used to express the nucleic acid sequences encoding the fusion proteins of the present invention. Suitable host cells include, but are not limited to prokaryotic cells such as bacteria, e.g. E.coli, eukaryotic host cells such as yeast cells, insect cells, plant cells or animal cells, preferably mammalian cells and, more preferably, human cells.

Further, the invention relates to a non-human organism transformed or transfected with a nucleic acid molecule as described above. Such transgenic organisms may be generated by known methods of genetic transfer including homologous recombination.

A further aspect of the present invention relates to a pharmaceutical or diagnostic composition comprising as the active agent at least one fusion protein, a respective nucleic acid encoding therefore, or a transformed or transfected cell, all as described herein.

Fusion proteins of the invention, respective nucleic acids encoding said fusion proteins, transformed or transfected cells useful for the production of said fusion proteins may be used in therapy, e.g., in the prophylaxis and/or treatment of disorders caused by, associated with and/or accompanied by dysfunction of TNF-SF cytokines, particularly proliferative disorders, such as tumors, e.g. solid or lymphatic tumors; infectious diseases; inflammatory diseases; metabolic diseases; autoimmune disorders, e.g. rheumatoid and/or arthritic diseases; degenerative diseases, e.g. neurodegenerative diseases such as multiple sclerosis; apoptosis-associated diseases or transplant rejections.

### EXAMPLES

### Example 1: Method for Large Scale Expression and Purification of Recombinant Multispecific/Bispecific TNF Superfamily Fusion Protein Assemblies

For large scale expression of the aforementioned multi-specific immune modulators of the invention, synthetic DNA cassettes encoding the necessary polypeptides (e.g. scTNFSF-Fc, antibody-HC, antibody-LC, VH-CH1-scTNFSF) are inserted into eukaryotic expression vectors comprising appropriate selection markers (e.g. a functional expression cassette comprising a blasticidin, puromycin, hygromycin or zeocin resistance gene) and genetic elements suitable to enhance the number of transcriptionally active insertion sites within the host cell genome, e.g. the human β-globin matrix attachment region (MAR). The sequence verified expression vectors are introduced by electroporation into suspension adapted Chinese Hamster Ovary cells (CHO-S, Invitrogen). Appropriate selection pressure was applied three days post transfection to the transfected cells. Surviving cells carrying the vector derived resistance genes are recovered by subsequent cultivation under selection pressure. Upon stable growth of the selected cell pools in chemically defined medium (PowerCHO-2 CD, Lonza, supplemented with 4 mM glutamine/glutamax) at 37° C. and 7% CO₂ atmosphere in an orbital shaker incubator (100 rpm, 50 mm shaking throw), the individual supernatants are analyzed by ELISA assays detecting the aforementioned proteins. Cell pools with the highest specific productivity are expanded in shake flasks for protein production (orbital shaker, 100 rpm, shaking throw 50 mm).

For lab-scale production, individual cell pools are cultured for 7-12 days in chemically defined medium (PowerCHO-2 CD, Lonza, supplemented with 4 mM glutamax) at 37° C. and 7% CO₂ atmosphere, either in shake flasks with orbital shaking (100 rpm, 55 mm shaking throw) or in a Wave bioreactor 20/50 EHT (GE Healthcare/Cytiva). The wave culture is started with a viable cell concentration of 0.3 ×10e6 cells/ml and the following settings (for five or ten liter): shaking frequency 18 rpm, shaking angle 7°, gas current 0.2-0.3 L/min, 7% CO₂, 36.5° C. During the wave run, the cell culture is fed twice with PowerFeed A (Lonza) with Lipids usually on day 3 (20 % feed) and on day 6 (30 % feed). After the second feed, shaking frequency is increased to 22 rpm and the shaking angle to 8°. The wave bioreactor is harvested between day 7 to day 10 when the cell viability drops below 80%. The culture supernatant containing bispecific TNFSF agonists is clarified using a depth filtration system (Millipore Millistak Pod MC0HC 0.054 m²), followed by sterile filtration of the clarified harvest using 0.22 µm bottle top filter (PES, Corning) and stored at 2-8° C. until further processing.

For affinity purification of the multi-specific immune modulators of the first and second aspect of the invention, a purification process on an ÄKTA chromatography system (GE Healthcare/Cytiva) is performed which makes use of the different properties of the aforementioned bispecific TNFSF Fc fusion proteins introduced by specific mutations in each of the both Fc-scaffolds used. First, MabSelect SuRe™ ProteinA (GE Healthcare/Cytiva) as solid phase affinity ligand is used which binds with high binding capacity to the Fc domain of the bispecific TNFSF agonist Fc fusion protein. Briefly, the sterile filtered clarified cell culture supernatant/harvest is loaded on a HiTrap MabSelect SuRe column (CV=5 ml) which was equilibrated in wash buffer 1 (20 mM Pi, 95 mM NaCl, pH 7.2) not exceeding a load of 10 mg fusion protein per ml column volume. The column is washed with 10 column volumes (10 CV) of wash buffer 1 followed by four column volumes (4 CV) of wash buffer 2 (20 mM Pi, 95 mM NaCl, pH 8.0) to deplete host-cell proteins and host-cell DNA. Also the homodimeric contaminant which is lacking a proteinA binding site is removed as it remains in the column flowthrough and does not bind to the column. After a series of washing steps, the protein is then eluted from the column with two column volumes elution buffer (20 mM Pi, 95 mM NaCl, pH 3.5). The eluate is collected in fractions and immediately neutralized with 1 M Tris-HCI pH 8.0 to neutral pH. The linear velocity is set to 150 cm/h and kept constant during the aforementioned affinity chromatography method.

In the case of the purification of the multi-specific immune modulators of the second aspect of the invention, the heterodimeric fusion protein present in the eluate is polished by a combination of SEC and ion-exchange chromatography.

The second affinity step for the purification of the multi-specific immune modulators of the first aspect of the invention employs KappaSelect™ Resin (GE Healthcare/Cytiva) which binds the CL-kappa domain of the Fab domain of the bispecific TNFSF agonist and depletes the homodimeric agonist Fc-fusion protein. Alternatively, the second affinity step employs Capture Select™ IgG-CH1 Resin (Thermo Scientific) which binds the CH1 domain of the Fab domain with high affinity. This also leads to the depletion of the homodimeric agonist Fc-fusion protein.The eluate of the first MabSelect SuRe™ ProteinA-based affinity chromatography is loaded either on the Capture Select IgG-CH1 (Thermo Scientific) or on KappaSelect Resin (GE Healthcare/Cytiva) (CV = 5 ml) equilibrated with wash buffer (PBS pH 7.4 = 10 mM Pi, 2.7 mM KCI, 140 mM NaCI), not exceeding 10 mg Fab per ml column volume. After a washing step with wash buffer (6 CV), the aforementioned bispecific TNFSF agonist was eluted with 2 CV elution buffer (0.1 M glycine, pH 3.5) and immediately neutralized with 1 M Tris-HCI pH 8.0 to neutral pH (0.4 CV). The protein amount of eluate fractions was quantified by OD 280 measurements and concentrated by ultrafiltration for subsequent size exclusion chromatography (SEC).

For the affinity purification of the multi-specific immune modulators of the third aspect of the invention, only the aforementioned CH1-based affinity purification is employed and the protein is polished by subsequent size exclusion chromatography.

Size exclusion chromatography (SEC) is performed on HiLoad 26/600 Superdex 200 pg or Superdex 200 Increase 10/300 GL columns (GE Healthcare/Cytiva) using an ÄKTA chromatography system. The columns are either equilibrated with phosphate buffered saline or an equivalent Tris based buffer system at neutral pH (pH 7.4).

The concentrated, affinity-purified protein is loaded onto the SEC column with the sample volume not exceeding 2% (v/v) of the column volume. A flow rate of 2.5 ml per min (HiLoad 26/600 Superdex 200 pg) or 0.5 ml per min (Superdex 200 Increase 10/300 GL) is applied and the elution profile monitored by absorbance at 280 nm. For determination of the apparent molecular weight of the purified protein under native conditions, the SEC columns are loaded with standard proteins of known molecular weight. Based on the elution volume of the standard proteins a calibration curve is plotted and the molecular weight of the purified protein is determined. The bispecific TNFRSF agonist fusion protein (SAB-format) from the first aspect of the invention and the bispecific TNFSF-ligand fusion protein of the second aspect of the invention elute from the Superdex SEC columns with an apparent molecular weight of around 150 kDa while the bispecific Fab-based fusion protein of the third aspect of the invention has an apparent molecular weight of around 100 kDa. HPLC, ELISA-based sandwich assays with both targets and TNFRSF reporter-cell based activity assays are used to determine the bispecific nature of the aforementioned bispecific TNFSF agonists.

### Example 2: Materials and Methods

### Cellular Activity of CD40 Agonistic Compounds

The cellular activity of CD40 agonists was assessed employing a CD40 Luciferase reporter gene assay from Promega (product no. JA2155). NF_{K}B-luc2-expressing U2OS cells (which constitutively express CD40 on their cell membrane) were plated in a 96-well plate and incubated for 16-20 hours at 37° C. prior to addition of CD40 agonists. Productive CD40 signaling induced by treatment with the agonistic compounds drives expression of firefly luciferase in the NF_{K}B-luc2 U2OS cells. After four hours of induction at 37° C., the luciferase assay reagent was added and luminescence (RLU) was measured (Tecan Infinite F500).

### Cellular Activity of CD27 Agonistic Compounds

The cellular activity of CD27 agonists was assessed employing a CD27 Luciferase reporter gene assay from Promega (product no. CS1979A25). NF_{K}B-luc2/CD27 Jurkat cells (which express CD27 on their cell membrane) were plated in a 96-well plate and incubated for 16-20 hours at 37° C. prior to addition of CD27 agonists. Productive CD27 signaling induced by treatment with the agonistic compounds drives expression of firefly luciferase in the NF_{K}B-luc2/CD27 Jurkat cells. After six hours of induction at 37° C., the luciferase assay reagent was added and luminescence (RLU) was measured (Tecan Infinite F500).

### Cellular Activity of GITR Agonistic Compounds

The cellular activity of GITR agonists was assessed employing a GITR Luciferase reporter gene assay from Promega (product no. CS184009). NF_{K}B-luc2/GITR Jurkat cells (which express GITR on their cell membrane) were plated in a 96-well plate and incubated shortly at 37° C. prior to addition of GITR agonists. Productive GITR signaling induced by treatment with the agonistic compounds drives expression of firefly luciferase in the NF_{K}B-luc2/GITR Jurkat cells. After five hours of induction at 37° C., the luciferase assay reagent was added and luminescence (RLU) was measured (Tecan Infinite F500).

### Cellular Activity of PD-L1-Targeting Compounds

The cellular activity of PD-L1-targeting compounds was assessed employing a PD-1/PD-L1 Luciferase reporter gene assay from Promega (product no. J1250). PD-L1 aAPC/CHO-K1 cells (cells expressing human PD-L1 and an engineered cell surface protein designed to activate cognate TCRs in an antigen-independent manner) are incubated for 16-20 hours at 37° C. prior to addition of PD-L1-targeting compounds and PD-1 effector cells. PD-1 effector cells are Jurkat T cells expressing human PD-1 and a luciferase reporter driven by an NFAT response element (NFAT-RE). When the two cell types are co-cultured, the PD-1/PD-L1 interaction inhibits TCR signaling and NFAT-RE-mediated luminescence. Addition of either an anti-PD-1 or anti-PD-L1 antibody that blocks the PD-1/PD-L1 interaction releases the inhibitory signal and results in TCR activation and NFAT-RE-mediated luminescence. After six hours of induction at 37° C., the luciferase assay reagent was added and luminescence (RLU) was measured (Tecan Infinite F500).

### T Cell Activation (Flow Cytometry)

To test the activity of CD137 agonists on primary human T cells, naïve pan T cells were isolated from PBMCs using indirect magnetic bead-based isolation kits (Cat. No. 130-094-131, Miltenyi). Purified T cells were labeled with Tag-it Violet™ Proliferation and Cell Tracking Dye (Biolegend), resuspended in medium (AIM-V w/ 5% human serum, Gibco) and stimulated with pre-coated anti-CD3 antibody 4h at 37° C., clone OKT3 , 1 µg/mL) or medium control. CD137 agonists (100 ng/ml) were added immediately. On day five, T cells were harvested and examined by flow cytometry.

### Stimulation of Immature Dendritic Cells (Flow Cytometry)

Monocytes were isolated from buffy coats from healthy human donors employing standard kits (Stem Cell). Differentiation of monocytes was achieved by adding 50 ng/ml GM-CSF for 3 days followed by 50 ng/ml GM-CSF + 50 ng/ml IL-4 for further 3 days. Cells were then treated with 100 ng/ml of the indicated CD40 agonists for 24 h followed by flow cytometry assessing CD86 and CD83 expression.

### Example 3: Sequences for Multi-Specific Immune Modulators

For all Fc-domain based heteromeric constructs, the knobs into holes hetero-dimerization technology was used with the S354C/T366W mutations in the CH3 domain of the knob chain and the corresponding Y349C/T366S/L368A/Y407V mutations in the CH3 domain of the hole chain (Carter, J Immunol Methods 248, 7-15 (2001)).

In order to abrogate binding to Fc gamma receptors the N297S mutation was introduced into the CH2-domain (“CH2s” in SEQ-IDs 28-31) of the knob and hole heavy chains. In another embodiment the Pro329Gly, Leu234Ala and Leu235Ala mutations can be introduced in the constant region of the knob and hole heavy chains according to the method described in International Patent Appl. Publ. No. WO 2012/130831 A1.

TABLE 1

Exemplary Hinge-linker sequences

| | |
|---|---|
| | Sequence |
| Hinge1 | GSSSSSSSSGSCDKTHTCPPC |
| Hinge2 | GSSSSSSSGSCDKTHTCPPC |
| Hinge3 | GSSSSSSGSCDKTHTCPPC |
| Hinge4 | GSSSSSGSCDKTHTCPPC |
| Hinge5 | GSSSGSCDKTHTCPPC |
| Hinge17 | GSGSSSGSCDKTHTCPPC |
| Hinge6 | GSSSSSSSSGSDKTHTCPPC |
| Hinge7 | GSSSSSSSGSDKTHTCPPC |
| Hinge8 | GSSSSSSGSDKTHTCPPC |
| Hinge9 | GSSSSSGSDKTHTCPPC |
| Hinge10 | GSSSGSDKTHTCPPC |
| Hinge11 | GSGSDKTHTCPPC |
| Hinge12 | GSGSGGGSDKTHTCPPC |
| Hinge13 | GSGSGGGSTHTCPPC |
| Hinge14 | GSGSTHTCPPC |
| Hinge15 | GSDKTHTCPPC |
| Hinge16 | GSGSSSGSDKTHTCPPC |

Hinge linkers 1-5 and 17 can be used in the construction of protein moieties of the second aspect of the invention. Hinge linkers 6 - 16 can be used in the construction of protein moieties of the first and the second aspect of the invention.

TABLE 2

Sequences of the invention An overview of important sequences of the invention is given in the Table 2 below and in FIG. 24

| | | |
|---|---|---|
| SEQ-ID | Name | Sequence |
| SEQ-ID:1 | Signal Peptide | METDTLLVFVLLVWVPAGNG |
| SEQ-ID:2 | Hinge1 | GSSSSSSSSGSCDKTHTCPPC |
| SEQ-ID:3 | Hinge2 | GSSSSSSSGSCDKTHTCPPC |
| SEQ-ID:4 | Hinge3 | GSSSSSSGSCDKTHTCPPC |
| SEQ-ID:5 | Hinge4 | GSSSSSGSCDKTHTCPPC |
| SEQ-ID:6 | Hinge5 | GSSSGSCDKTHTCPPC |
| SEQ-ID:78 | Hinge17 | GSGSSSGSCDKTHTCPPC |
| SEQ-ID:7 | Hinge6 | GSSSSSSSSGSDKTHTCPPC |
| SEQ-ID:8 | Hinge7 | GSSSSSSSGSDKTHTCPPC |
| SEQ-ID:9 | Hinge8 | GSSSSSSGSDKTHTCPPC |
| SEQ-ID:10 | Hinge9 | GSSSSSGSDKTHTCPPC |
| SEQ-ID:11 | Hinge10 | GSSSGSDKTHTCPPC |
| SEQ-ID:12 | Hinge11 | GSGSDKTHTCPPC |
| SEQ-ID:79 | Hinge12 | GSGSGGGSDKTHTCPPC |
| SEQ-ID:80 | Hinge13 | GSGSGGGSTHTCPPC |
| SEQ-ID:81 | Hinge14 | GSGSTHTCPPC |
| SEQ-ID:82 | Hinge15 | GSDKTHTCPPC |
| SEQ-ID:83 | Hinge16 | GSGSSSGSDKTHTCPPC |
| SEQ-ID:13 | Linker-1 | DKTHGSGSSSSSS |
| SEQ-ID:14 | Linker-2 | DKTHGSGSSSS |
| SEQ-ID:15 | Linker-3 | DKTHGSGS |
| SEQ-ID:16 | Linker-4 | GSGSSSSSS |
| SEQ-ID:17 | Linker-5 | GSGSSS |
| SEQ-ID:18 | Linker-6 | GSGS |
| SEQ-ID:19 | Linker-7 | GGGSGGGS |
| SEQ-ID:20 | Fc-N297S-knob | PAPELLGGPSVFLFPPKPKDTLMISRTPEVTCVWDVSHEDPEVK FNWYVDGVEVHNAKTKPREEQYSSTYRVVSVLTVLHQDWLNGK EYKCKVSNKALPAPIEKTISKAKGQPREPQVYTLPPCRDELTKNQ VSLWCLVKGFYPSDIAVEWESNGQPENNYKTTPPVLDSDGSFFL YSKLTVDKSRWQQGNVFSCSVMHEALHNHYTQKSLSLSPGK |
| SEQ-ID:21 | Fc-N297S-hole | PAPELLGGPSVFLFPPKPKDTLMISRTPEVTCVWDVSHEDPEVK FNWYVDGVEVHNAKTKPREEQYSSTYRVVSVLTVLHQDWLNGK EYKCKVSNKALPAPIEKTISKAKGQPREPQVCTLPPSRDELTKNQ VSLSCAVKGFYPSDIAVEWESNGQPENNYKTTPPVLDSDGSFFLV SKLTVDKSRWQQGNVFSCSVMHEALHNHYTQKSLSLSPGK |
| SEQ-ID:22 | Fc-Knob-Variant-2 | PAPEAAGGPSVFLFPPKPKDTLMISRTPEVTCVWDVSHEDPEVK FNWYVDGVEVHNAKTKPREEQYNSTYRVVSVLTVLHQDWLNGK EYKCKVSNKALGAPIEKTISKAKGQPREPQVYTLPPCRDELTKNQ VSLWCLVKGFYPSDIAVEWESNGQPENNYKTTPPVLDSDGSFFL YSKLTVDKSRWQQGNVFSCSVMHEALHNHYTQKSLSLSPGK |
| SEQ-ID:23 | Fc-hole-Variant-2 | PAPEAAGGPSVFLFPPKPKDTLMISRTPEVTCVWDVSHEDPEVK FNWYVDGVEVHNAKTKPREEQYNSTYRVVSVLTVLHQDWLNGK EYKCKVSNKALGAPIEKTISKAKGQPREPQVCTLPPSRDELTKNQ VSLSCAVKGFYPSDIAVEWESNGQPENNYKTTPPVLDSDGSFFLV SKLTVDKSRWQQGNVFSCSVMHEALHNHYTQKSLSLSPGK |
| SEQ-ID:24 | Fc-knob-Variant-3 | PAPPVAGPSVFLFPPKPKDTLMISRTPEVTCVWDVSHEDPEVKF NWYVDGVEVHNAKTKPREEQYNSTYRVVSVLTVLHQDWLNGKE YKCKVSNKGLPSSIEKTISKAKGQPREPQVYTLPPCRDELTKNQV SLWCLVKGFYPSDIAVEWESNGQPENNYKTTPPVLDSDGSFFLY SKLTVDKSRWQQGNVFSCSVMHEALHNHYTQKSLSLSPGK |
| SEQ-ID:25 | Fc-hole-Variant-3 | PAPPVAGPSVFLFPPKPKDTLMISRTPEVTCVWDVSHEDPEVKF NWYVDGVEVHNAKTKPREEQYNSTYRWSVLTVLHQDWLNGKE YKCKVSNKGLPSSIEKTISKAKGQPREPQVCTLPPSRDELTKNQV SLSCAVKGFYPSDIAVEWESNGQPENNYKTTPPVLDSDGSFFLVS KLTVDKSRWQQGNVFSCSVMHEALHNHYTQKSLSLSPGK |
| SEQ-ID:109 | Fc-N297S | PAPELLGGPSVFLFPPKPKDTLMISRTPEVTCVWDVSHEDPEVK FNWYVDGVEVHNAKTKPREEQYSSTYRVVSVLTVLHQDWLNGK EYKCKVSNKALPAPIEKTISKAKGQPREPQVYTLPPSRDELTKNQV SLTCLVKGFYPSDIAVEWESNGQPENNYKTTPPVLDSDGSFFLYS KLTVDKSRWQQGNVFSCSVMHEALHNHYTQKSLSLSPGK |
| SEQ-ID:110 | Fc-WT | PAPELLGGPSVFLFPPKPKDTLMISRTPEVTCVWDVSHEDPEVK FNWYVDGVEVHNAKTKPREEQYNSTYRVVSVLTVLHQDWLNGK EYKCKVSNKALPAPIEKTISKAKGQPREPQVYTLPPSRDELTKNQV SLTCLVKGFYPSDIAVEWESNGQPENNYKTTPPVLDSDGSFFLYS KLTVDKSRWQQGNVFSCSVMHEALHNHYTQKSLSLSPGK |
| SEQ-ID:26 | IGG1-CL-kappa | TVAAPSVFIFPPSDEQLKSGTASVVCLLNNFYPREAKVQWKVDNA LQSGNSQESVTEQDSKDSTYSLSSTLTLSKADYEKHKVYACEVTH QGLSSPVTKSFNRGEC |
| SEQ-ID:27 | IGG1-CH1 | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSC |
| SEQ-ID:28 | IGG1-CH1CH2sCH3 -knob | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPREEQ YSSTYRWSVLTVLHQDWLNGKEYKCKVSNKALPAPIEKTISKAK GQPREPQVYTLPPCRDELTKNQVSLWCLVKGFYPSDIAVEWESN GQPENNYKTTPPVLDSDGSFFLYSKLTVDKSRWQQGNVFSCSV MHEALHNHYTQKSLSLSPGK |
| SEQ-ID:29 | IGG1-CH1CH2sCH3 -RF-knob | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPREEQ YSSTYRWSVLTVLHQDWLNGKEYKCKVSNKALPAPIEKTISKAK GQPREPQVYTLPPCRDELTKNQVSLWCLVKGFYPSDIAVEWESN GQPENNYKTTPPVLDSDGSFFLYSKLTVDKSRWQQGNVFSCSV MHEALHNRFTQKSLSLSPGK |
| SEQ-ID:30 | IGG1-CH1CH2sCH3 -hole | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPREEQ YSSTYRWSVLTVLHQDWLNGKEYKCKVSNKALPAPIEKTISKAK GQPREPQVCTLPPSRDELTKNQVSLSCAVKGFYPSDIAVEWESN GQPENNYKTTPPVLDSDGSFFLVSKLTVDKSRWQQGNVFSCSV MHEALHNHYTQKSLSLSPGK |
| SEQ-ID:31 | IGG1-CH1CH2sCH3 -RF-hole | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPREEQ YSSTYRWSVLTVLHQDWLNGKEYKCKVSNKALPAPIEKTISKAK GQPREPQVCTLPPSRDELTKNQVSLSCAVKGFYPSDIAVEWESN GQPENNYKTTPPVLDSDGSFFLVSKLTVDKSRWQQGNVFSCSV MHEALHNRFTQKSLSLSPGK |
| SEQ-ID:111 | IGG1-CH1CH2CH3-RF | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPREEQ YNSTYRVVSVLTVLHQDWLNGKEYKCKVSNKALPAPIEKTISKAK GQPREPQVYTLPPSRDELTKNQVSLTCLVKGFYPSDIAVEWESN GQPENNYKTTPPVLDSDGSFFLYSKLTVDKSRWQQGNVFSCSV MHEALHNRFTQKSLSLSPGK |
| SEQ-ID:112 | IGG1-CH1CH2sCH3 -RF | ASTKGPSVFPLAPSSKSTSGGTAALGCLVKDYFPEPVTVSWNSG ALTSGVHTFPAVLQSSGLYSLSSVVTVPSSSLGTQTYICNVNHKP SNTKVDKKVEPKSCDKTHTCPPCPAPELLGGPSVFLFPPKPKDTL MISRTPEVTCVVVDVSHEDPEVKFNWYVDGVEVHNAKTKPREEQ YSSTYRWSVLTVLHQDWLNGKEYKCKVSNKALPAPIEKTISKAK GQPREPQVYTLPPSRDELTKNQVSLTCLVKGFYPSDIAVEWESN GQPENNYKTTPPVLDSDGSFFLYSKLTVDKSRWQQGNVFSCSV MHEALHNRFTQKSLSLSPGK |
| SEQ-ID:32 | scCD40L-Fc-knob_a | QIAAHVISEASSKTTSVLQWAEKGYYTMSNNLVTLENGKQLTVKR QGLYYIYAQVTFCSNREASSQAPFIASLsLKSPGRFERILLRAANTH SSAKPCGQQSIHLGGVFELQPGASVFVNVTDPSQVSHGTGFTSF GLLKLGSGSGNGSQIAAHVISEASSKTTSVLQWAEKGYYTMSNNL VTLENGKQLTVKRQGLYYIYAQVTFCSNREASSQAPFIASLsLKSP GRFERILLRAANTHSSAKPCGQQSIHLGGVFELQPGASVFVNVTD PSQVSHGTGFTSFGLLKLGSGSGNGSQIAAHVISEASSKTTSVLQ WAEKGYYTMSNNLVTLENGKQLTVKRQGLYYIYAQVTFCSNREA SSQAPFIASLsLKSPGRFERILLRAANTHSSAKPCGQQSIHLGGVF ELQPGASVFVNVTDPSQVSHGTGFTSFGLLKLGSGSSSGSCDKT HTCPPCPAPELLGGPSVFLFPPKPKDTLMISRTPEVTCVWDVSH EDPEVKFNWYVDGVEVHNAKTKPREEQYSSTYRVVSVLTVLHQD WLNGKEYKCKVSNKALPAPIEKTISKAKGQPREPQVYTLPPCRDE LTKNQVSLWCLVKGFYPSDIAVEWESNGQPENNYKTTPPVLDSD GSFFLYSKLTVDKSRWQQGNVFSCSVMHEALHNHYTQKSLSLSP GK |
| SEQ-ID:33 | scCD40L-Fc-knob_b | QIAAHVISEASSKTTSVLQWAEKGYYTMSNNLVTLENGKQLTVKR QGLYYIYAQVTFCSNREASSQAPFIASLSLKSPGRFERILLRAANT HSSAKPCGQQSIHLGGVFELQPGASVFVNVTDPSQVSHGTGFTS FGLLKLGSGSGNGSQIAAHVISEASSKTTSVLQWAEKGYYTMSNN LVTLENGKQLTVKRQGLYYIYAQVTFCSNREASSQAPFIASLSLKS PGRFERILLRAANTHSSAKPCGQQSIHLGGVFELQPGASVFVNVT DPSQVSHGTGFTSFGLLKLGSGSGNGSQIAAHVISEASSKTTSVL QWAEKGYYTMSNNLVTLENGKQLTVKRQGLYYIYAQVTFCSNRE ASSQAPFIASLSLKSPGRFERILLRAANTHSSAKPCGQQSIHLGGV FELQPGASVFVNVTDPSQVSHGTGFTSFGLLKLGSGSDKTHTCP PCPAPELLGGPSVFLFPPKPKDTLMISRTPEVTCVWDVSHEDPE VKFNWYVDGVEVHNAKTKPREEQYSSTYRVVSVLTVLHQDWLN GKEYKCKVSNKALPAPIEKTISKAKGQPREPQVYTLPPCRDELTK NQVSLWCLVKGFYPSDIAVEWESNGQPENNYKTTPPVLDSDGSF FLYSKLTVDKSRWQQGNVFSCSVMHEALHNHYTQKSLSLSPGK |
| SEQ-ID:84 | scCD40L-Fc-knob_c | QIAAHVISEASSKTTSVLQWAEKGYYTMSNNLVTLENGKQLTVKR QGLYYIYAQVTFCSNREASSQAPFIASLSLKSPGRFERILLRAANT HSSAKPCGQQSIHLGGVFELQPGASVFVNVTDPSQVSHGTGFTS FGLLKLGSGSGNGSQIAAHVISEASSKTTSVLQWAEKGYYTMSNN LVTLENGKQLTVKRQGLYYIYAQVTFCSNREASSQAPFIASLSLKS PGRFERILLRAANTHSSAKPCGQQSIHLGGVFELQPGASVFVNVT DPSQVSHGTGFTSFGLLKLGSGSGNGSQIAAHVISEASSKTTSVL QWAEKGYYTMSNNLVTLENGKQLTVKRQGLYYIYAQVTFCSNRE ASSQAPFIASLSLKSPGRFERILLRAANTHSSAKPCGQQSIHLGGV FELQPGASVFVNVTDPSQVSHGTGFTSFGLLKLGSGSSSGSDKT HTCPPCPAPELLGGPSVFLFPPKPKDTLMISRTPEVTCVWDVSH EDPEVKFNWYVDGVEVHNAKTKPREEQYSSTYRVVSVLTVLHQD WLNGKEYKCKVSNKALPAPIEKTISKAKGQPREPQVYTLPPCRDE LTKNQVSLWCLVKGFYPSDIAVEWESNGQPENNYKTTPPVLDSD GSFFLYSKLTVDKSRWQQGNVFSCSVMHEALHNHYTQKSLSLSP GK |
| SEQ-ID:34 | scCD40L-Fc-hole_a | QIAAHVISEASSKTTSVLQWAEKGYYTMSNNLVTLENGKQLTVKR QGLYYIYAQVTFCSNREASSQAPFIASLsLKSPGRFERILLRAANTH SSAKPCGQQSIHLGGVFELQPGASVFVNVTDPSQVSHGTGFTSF GLLKLGSGSGNGSQIAAHVISEASSKTTSVLQWAEKGYYTMSNNL VTLENGKQLTVKRQGLYYIYAQVTFCSNREASSQAPFIASLsLKSP GRFERILLRAANTHSSAKPCGQQSIHLGGVFELQPGASVFVNVTD PSQVSHGTGFTSFGLLKLGSGSGNGSQIAAHVISEASSKTTSVLQ WAEKGYYTMSNNLVTLENGKQLTVKRQGLYYIYAQVTFCSNREA SSQAPFIASLsLKSPGRFERILLRAANTHSSAKPCGQQSIHLGGVF ELQPGASVFVNVTDPSQVSHGTGFTSFGLLKLGSGSSSGSCDKT HTCPPCPAPELLGGPSVFLFPPKPKDTLMISRTPEVTCVWDVSH EDPEVKFNWYVDGVEVHNAKTKPREEQYSSTYRVVSVLTVLHQD WLNGKEYKCKVSNKALPAPIEKTISKAKGQPREPQVCTLPPSRDE LTKNQVSLSCAVKGFYPSDIAVEWESNGQPENNYKTTPPVLDSD GSFFLVSKLTVDKSRWQQGNVFSCSVMHEALHNHYTQKSLSLSP GK |
| SEQ-ID:35 | scCD40L-Fc-hole_b | QIAAHVISEASSKTTSVLQWAEKGYYTMSNNLVTLENGKQLTVKR QGLYYIYAQVTFCSNREASSQAPFIASLsLKSPGRFERILLRAANTH SSAKPCGQQSIHLGGVFELQPGASVFVNVTDPSQVSHGTGFTSF GLLKLGSGSGNGSQIAAHVISEASSKTTSVLQWAEKGYYTMSNNL VTLENGKQLTVKRQGLYYIYAQVTFCSNREASSQAPFIASLsLKSP GRFERILLRAANTHSSAKPCGQQSIHLGGVFELQPGASVFVNVTD PSQVSHGTGFTSFGLLKLGSGSGNGSQIAAHVISEASSKTTSVLQ WAEKGYYTMSNNLVTLENGKQLTVKRQGLYYIYAQVTFCSNREA SSQAPFIASLsLKSPGRFERILLRAANTHSSAKPCGQQSIHLGGVF ELQPGASVFVNVTDPSQVSHGTGFTSFGLLKLGSGSDKTHTCPP CPAPELLGGPSVFLFPPKPKDTLMISRTPEVTCVWDVSHEDPEV KFNWYVDGVEVHNAKTKPREEQYSSTYRVVSVLTVLHQDWLNG KEYKCKVSNKALPAPIEKTISKAKGQPREPQVCTLPPSRDELTKN QVSLSCAVKGFYPSDIAVEWESNGQPENNYKTTPPVLDSDGSFF LVSKLTVDKSRWQQGNVFSCSVMHEALHNHYTQKSLSLSPGK |
| SEQ-ID:36 | scCD27L-Fc-knob_a | QSLGWDVAELQLNHTGPQQDPRLYWQGGPALGRSFLHGPELDK GQLRIHRDGIYMVHIQVTLAICSSTTASRHHPTTLAVGICSPASRSI SLLRLSFHQGCTIASQRLTPLARGDTLCTNLTGTLLPSRNTDETFF GVQWVRPGSGSGNGSESLGWDVAELQLNHTGPQQDPRLYWQG GPALGRSFLHGPELDKGQLRIHRDGIYMVHIQVTLAICSSTTASRH HPTTLAVGICSPASRSISLLRLSFHQGCTIASQRLTPLARGDTLCTN LTGTLLPSRNTDETFFGVQWVRPGSGSGNGSESLGWDVAELQL NHTGPQQDPRLYWQGGPALGRSFLHGPELDKGQLRIHRDGIYMV HIQVTLAICSSTTASRHHPTTLAVGICSPASRSISLLRLSFHQGCTIA SQRLTPLARGDTLCTNLTGTLLPSRNTDETFFGVQWVRPGSGSS SGSCDKTHTCPPCPAPELLGGPSVFLFPPKPKDTLMISRTPEVTC VVVDVSHEDPEVKFNWYVDGVEVHNAKTKPREEQYSSTYRVVSV LTVLHQDWLNGKEYKCKVSNKALPAPIEKTISKAKGQPREPQVYT LPPCRDELTKNQVSLWCLVKGFYPSDIAVEWESNGQPENNYKTT PPVLDSDGSFFLYSKLTVDKSRWQQGNVFSCSVMHEALHNHYTQ KSLSLSPGK |
| SEQ-ID:37 | scCD27L-Fc-knob_b | QSLGWDVAELQLNHTGPQQDPRLYWQGGPALGRSFLHGPELDK GQLRIHRDGIYMVHIQVTLAICSSTTASRHHPTTLAVGICSPASRSI SLLRLSFHQGCTIASQRLTPLARGDTLCTNLTGTLLPSRNTDETFF GVQWVRPGSGSGNGSESLGWDVAELQLNHTGPQQDPRLYWQG GPALGRSFLHGPELDKGQLRIHRDGIYMVHIQVTLAICSSTTASRH HPTTLAVGICSPASRSISLLRLSFHQGCTIASQRLTPLARGDTLCTN LTGTLLPSRNTDETFFGVQWVRPGSGSGNGSESLGWDVAELQL NHTGPQQDPRLYWQGGPALGRSFLHGPELDKGQLRIHRDGIYMV HIQVTLAICSSTTASRHHPTTLAVGICSPASRSISLLRLSFHQGCTIA SQRLTPLARGDTLCTNLTGTLLPSRNTDETFFGVQWVRPGSGSD KTHTCPPCPAPELLGGPSVFLFPPKPKDTLMISRTPEVTCVWDV SHEDPEVKFNWYVDGVEVHNAKTKPREEQYSSTYRVVSVLTVLH QDWLNGKEYKCKVSNKALPAPIEKTISKAKGQPREPQVYTLPPCR DELTKNQVSLWCLVKGFYPSDIAVEWESNGQPENNYKTTPPVLD SDGSFFLYSKLTVDKSRWQQGNVFSCSVMHEALHNHYTQKSLSL SPGK |
| SEQ-ID:38 | scCD27L-Fc-hole_a | QSLGWDVAELQLNHTGPQQDPRLYWQGGPALGRSFLHGPELDK GQLRIHRDGIYMVHIQVTLAICSSTTASRHHPTTLAVGICSPASRSI SLLRLSFHQGCTIASQRLTPLARGDTLCTNLTGTLLPSRNTDETFF GVQWVRPGSGSGNGSESLGWDVAELQLNHTGPQQDPRLYWQG GPALGRSFLHGPELDKGQLRIHRDGIYMVHIQVTLAICSSTTASRH HPTTLAVGICSPASRSISLLRLSFHQGCTIASQRLTPLARGDTLCTN LTGTLLPSRNTDETFFGVQWVRPGSGSGNGSESLGWDVAELQL NHTGPQQDPRLYWQGGPALGRSFLHGPELDKGQLRIHRDGIYMV HIQVTLAICSSTTASRHHPTTLAVGICSPASRSISLLRLSFHQGCTIA SQRLTPLARGDTLCTNLTGTLLPSRNTDETFFGVQWVRPGSGSS SGSCDKTHTCPPCPAPELLGGPSVFLFPPKPKDTLMISRTPEVTC VVVDVSHEDPEVKFNWYVDGVEVHNAKTKPREEQYSSTYRWSV LTVLHQDWLNGKEYKCKVSNKALPAPIEKTISKAKGQPREPQVCT LPPSRDELTKNQVSLSCAVKGFYPSDIAVEWESNGQPENNYKTT PPVLDSDGSFFLVSKLTVDKSRWQQGNVFSCSVMHEALHNHYTQ KSLSLSPGK |
| SEQ-ID:39 | scCD27L-Fc-hole_b | QSLGWDVAELQLNHTGPQQDPRLYWQGGPALGRSFLHGPELDK GQLRIHRDGIYMVHIQVTLAICSSTTASRHHPTTLAVGICSPASRSI SLLRLSFHQGCTIASQRLTPLARGDTLCTNLTGTLLPSRNTDETFF GVQWVRPGSGSGNGSESLGWDVAELQLNHTGPQQDPRLYWQG GPALGRSFLHGPELDKGQLRIHRDGIYMVHIQVTLAICSSTTASRH HPTTLAVGICSPASRSISLLRLSFHQGCTIASQRLTPLARGDTLCTN LTGTLLPSRNTDETFFGVQWVRPGSGSGNGSESLGWDVAELQL NHTGPQQDPRLYWQGGPALGRSFLHGPELDKGQLRIHRDGIYMV HIQVTLAICSSTTASRHHPTTLAVGICSPASRSISLLRLSFHQGCTIA SQRLTPLARGDTLCTNLTGTLLPSRNTDETFFGVQWVRPGSGSD KTHTCPPCPAPELLGGPSVFLFPPKPKDTLMISRTPEVTCVWDV SHEDPEVKFNWYVDGVEVHNAKTKPREEQYSSTYRVVSVLTVLH QDWLNGKEYKCKVSNKALPAPIEKTISKAKGQPREPQVCTLPPSR DELTKNQVSLSCAVKGFYPSDIAVEWESNGQPENNYKTTPPVLD SDGSFFLVSKLTVDKSRWQQGNVFSCSVMHEALHNHYTQKSLSL SPGK |
| SEQ-ID:40 | scGITRL-Fc-knob_a | QPCMAKFGPLPSKWQMASSEPPCVNKVSDWKLEILQNGLYLIYG QVAPNANYNDVAPFEVRLYKNKDMIQTLTNKSKIQNVGGTYELHV GDTIDLIFNSEHQVLKNNTYWGIILLANPQFISGSGSGNGSEPCMA KFGPLPSKWQMASSEPPCVNKVSDWKLEILQNGLYLIYGQVAPN ANYNDVAPFEVRLYKNKDMIQTLTNKSKIQNVGGTYELHVGDTIDL IFNSEHQVLKNNTYWGIILLANPQFISGSGSGNGSEPCMAKFGPLP SKWQMASSEPPCVNKVSDWKLEILQNGLYLIYGQVAPNANYNDV APFEVRLYKNKDMIQTLTNKSKIQNVGGTYELHVGDTIDLIFNSEH QVLKNNTYWGIILLANPQFISGSGSSSGSCDKTHTCPPCPAPELL GGPSVFLFPPKPKDTLMISRTPEVTCVVVDVSHEDPEVKFNWYVD GVEVHNAKTKPREEQYSSTYRVVSVLTVLHQDWLNGKEYKCKVS NKALPAPIEKTISKAKGQPREPQVYTLPPCRDELTKNQVSLWCLV KGFYPSDIAVEWESNGQPENNYKTTPPVLDSDGSFFLYSKLTVDK SRWQQGNVFSCSVMHEALHNHYTQKSLSLSPGK |
| SEQ-ID:41 | scGITRL-Fc-knob_b | QPCMAKFGPLPSKWQMASSEPPCVNKVSDWKLEILQNGLYLIYG QVAPNANYNDVAPFEVRLYKNKDMIQTLTNKSKIQNVGGTYELHV GDTIDLIFNSEHQVLKNNTYWGIILLANPQFISGSGSGNGSEPCMA KFGPLPSKWQMASSEPPCVNKVSDWKLEILQNGLYLIYGQVAPN ANYNDVAPFEVRLYKNKDMIQTLTNKSKIQNVGGTYELHVGDTIDL IFNSEHQVLKNNTYWGIILLANPQFISGSGSGNGSEPCMAKFGPLP SKWQMASSEPPCVNKVSDWKLEILQNGLYLIYGQVAPNANYNDV APFEVRLYKNKDMIQTLTNKSKIQNVGGTYELHVGDTIDLIFNSEH QVLKNNTYWGIILLANPQFISGSGSDKTHTCPPCPAPELLGGPSVF LFPPKPKDTLMISRTPEVTCVVVDVSHEDPEVKFNWYVDGVEVH NAKTKPREEQYSSTYRVVSVLTVLHQDWLNGKEYKCKVSNKALP APIEKTISKAKGQPREPQVYTLPPCRDELTKNQVSLWCLVKGFYP SDIAVEWESNGQPENNYKTTPPVLDSDGSFFLYSKLTVDKSRWQ QGNVFSCSVMHEALHNHYTQKSLSLSPGK |
| SEQ-ID:42 | scGITRL-Fc-hole_a | QPCMAKFGPLPSKWQMASSEPPCVNKVSDWKLEILQNGLYLIYG QVAPNANYNDVAPFEVRLYKNKDMIQTLTNKSKIQNVGGTYELHV GDTIDLIFNSEHQVLKNNTYWGIILLANPQFISGSGSGNGSEPCMA KFGPLPSKWQMASSEPPCVNKVSDWKLEILQNGLYLIYGQVAPN ANYNDVAPFEVRLYKNKDMIQTLTNKSKIQNVGGTYELHVGDTIDL IFNSEHQVLKNNTYWGIILLANPQFISGSGSGNGSEPCMAKFGPLP SKWQMASSEPPCVNKVSDWKLEILQNGLYLIYGQVAPNANYNDV APFEVRLYKNKDMIQTLTNKSKIQNVGGTYELHVGDTIDLIFNSEH QVLKNNTYWGIILLANPQFISGSGSSSGSCDKTHTCPPCPAPELL GGPSVFLFPPKPKDTLMISRTPEVTCVVVDVSHEDPEVKFNWYVD GVEVHNAKTKPREEQYSSTYRVVSVLTVLHQDWLNGKEYKCKVS NKALPAPIEKTISKAKGQPREPQVCTLPPSRDELTKNQVSLSCAVK GFYPSDIAVEWESNGQPENNYKTTPPVLDSDGSFFLVSKLTVDKS RWQQGNVFSCSVMHEALHNHYTQKSLSLSPGK |
| SEQ-ID:43 | scGITRL-Fc-hole_b | QPCMAKFGPLPSKWQMASSEPPCVNKVSDWKLEILQNGLYLIYG QVAPNANYNDVAPFEVRLYKNKDMIQTLTNKSKIQNVGGTYELHV GDTIDLIFNSEHQVLKNNTYWGIILLANPQFISGSGSGNGSEPCMA KFGPLPSKWQMASSEPPCVNKVSDWKLEILQNGLYLIYGQVAPN ANYNDVAPFEVRLYKNKDMIQTLTNKSKIQNVGGTYELHVGDTIDL IFNSEHQVLKNNTYWGIILLANPQFISGSGSGNGSEPCMAKFGPLP SKWQMASSEPPCVNKVSDWKLEILQNGLYLIYGQVAPNANYNDV APFEVRLYKNKDMIQTLTNKSKIQNVGGTYELHVGDTIDLIFNSEH QVLKNNTYWGIILLANPQFISGSGSDKTHTCPPCPAPELLGGPSVF LFPPKPKDTLMISRTPEVTCVVVDVSHEDPEVKFNWYVDGVEVH NAKTKPREEQYSSTYRVVSVLTVLHQDWLNGKEYKCKVSNKALP APIEKTISKAKGQPREPQVCTLPPSRDELTKNQVSLSCAVKGFYP SDIAVEWESNGQPENNYKTTPPVLDSDGSFFLVSKLTVDKSRWQ QGNVFSCSVMHEALHNHYTQKSLSLSPGK |
| SEQ-ID:85 | scCD137L-V1-Fc-knob_a | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVGSGSGNGSQGMFAQLVAQNVLLIDG PLSWYSDPGLAGVSLTGGLSYKEDTKELVVAKAGVYYVFFQLELR RWAGEGSGSVSLALHLQPLRSAAGAAALALTVDLPPASSEARNS AFGFQGRLLHLSAGQRLGVHLHTEARARHAWQLTQGATVLGLFR VGSGSGNGSQGMFAQLVAQNVLLIDGPLSWYSDPGLAGVSLTG GLSYKEDTKELVVAKAGVYYVFFQLELRRVVAGEGSGSVSLALHL QPLRSAAGAAALALTVDLPPASSEARNSAFGFQGRLLHLSAGQRL GVHLHTEARARHAWQLTQGATVLGLFRVGSGSSSGSCDKTHTC PPCPAPELLGGPSVFLFPPKPKDTLMISRTPEVTCVWDVSHEDP EVKFNWYVDGVEVHNAKTKPREEQYSSTYRVVSVLTVLHQDWLN GKEYKCKVSNKALPAPIEKTISKAKGQPREPQVYTLPPCRDELTK NQVSLWCLVKGFYPSDIAVEWESNGQPENNYKTTPPVLDSDGSF FLYSKLTVDKSRWQQGNVFSCSVMHEALHNHYTQKSLSLSPGK |
| SEQ-ID:86 | scCD137L-V1-Fc-knob_b | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVGSGSGNGSQGMFAQLVAQNVLLIDG PLSWYSDPGLAGVSLTGGLSYKEDTKELVVAKAGVYYVFFQLELR RWAGEGSGSVSLALHLQPLRSAAGAAALALTVDLPPASSEARNS AFGFQGRLLHLSAGQRLGVHLHTEARARHAWQLTQGATVLGLFR VGSGSGNGSQGMFAQLVAQNVLLIDGPLSWYSDPGLAGVSLTG GLSYKEDTKELVVAKAGVYYVFFQLELRRVVAGEGSGSVSLALHL QPLRSAAGAAALALTVDLPPASSEARNSAFGFQGRLLHLSAGQRL GVHLHTEARARHAWQLTQGATVLGLFRVGSGSDKTHTCPPCPAP ELLGGPSVFLFPPKPKDTLMISRTPEVTCVVVDVSHEDPEVKFNW YVDGVEVHNAKTKPREEQYSSTYRVVSVLTVLHQDWLNGKEYKC KVSNKALPAPIEKTISKAKGQPREPQVYTLPPCRDELTKNQVSLW CLVKGFYPSDIAVEWESNGQPENNYKTTPPVLDSDGSFFLYSKLT VDKSRWQQGNVFSCSVMHEALHNHYTQKSLSLSPGK |
| SEQ-ID:87 | scCD137L-V1-Fc-hole_a | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVGSGSGNGSQGMFAQLVAQNVLLIDG PLSWYSDPGLAGVSLTGGLSYKEDTKELVVAKAGVYYVFFQLELR RWAGEGSGSVSLALHLQPLRSAAGAAALALTVDLPPASSEARNS AFGFQGRLLHLSAGQRLGVHLHTEARARHAWQLTQGATVLGLFR VGSGSGNGSQGMFAQLVAQNVLLIDGPLSWYSDPGLAGVSLTG GLSYKEDTKELVVAKAGVYYVFFQLELRRVVAGEGSGSVSLALHL QPLRSAAGAAALALTVDLPPASSEARNSAFGFQGRLLHLSAGQRL GVHLHTEARARHAWQLTQGATVLGLFRVGSGSSSGSCDKTHTC PPCPAPELLGGPSVFLFPPKPKDTLMISRTPEVTCVWDVSHEDP EVKFNWYVDGVEVHNAKTKPREEQYSSTYRVVSVLTVLHQDWLN GKEYKCKVSNKALPAPIEKTISKAKGQPREPQVCTLPPSRDELTK NQVSLSCAVKGFYPSDIAVEWESNGQPENNYKTTPPVLDSDGSF FLVSKLTVDKSRWQQGNVFSCSVMHEALHNHYTQKSLSLSPGK |
| SEQ-ID:88 | scCD137L-V1-Fc-hole_b | RWAGEGSGSVSLALHLQPLRSAAGAAALALTVDLPPASSEARNS AFGFQGRLLHLSAGQRLGVHLHTEARARHAWQLTQGATVLGLFR VGSGSGNGSQGMFAQLVAQNVLLIDGPLSWYSDPGLAGVSLTG GLSYKEDTKELVVAKAGVYYVFFQLELRRVVAGEGSGSVSLALHL QPLRSAAGAAALALTVDLPPASSEARNSAFGFQGRLLHLSAGQRL GVHLHTEARARHAWQLTQGATVLGLFRVGSGSGNGSQGMFAQL VAQNVLLIDGPLSWYSDPGLAGVSLTGGLSYKEDTKELVVAKAGV YYVFFQLELRRVVAGEGSGSVSLALHLQPLRSAAGAAALALTVDL PPASSEARNSAFGFQGRLLHLSAGQRLGVHLHTEARARHAWQLT QGATVLGLFRVGSGSDKTHTCPPCPAPELLGGPSVFLFPPKPKDT LMISRTPEVTCVVVDVSHEDPEVKFNWYVDGVEVHNAKTKPREE QYSSTYRVVSVLTVLHQDWLNGKEYKCKVSNKALPAPIEKTISKA KGQPREPQVCTLPPSRDELTKNQVSLSCAVKGFYPSDIAVEWES NGQPENNYKTTPPVLDSDGSFFLVSKLTVDKSRWQQGNVFSCSV MHEALHNHYTQKSLSLSPGK |
| SEQ-ID:89 | scCD137L-V2-Fc-knob_a | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGNGSGSGMFAQLVAQNVLLID GPLSWYSDPGLAGVSLTGGLSYKEDTKELVVAKAGVYYVFFQLEL RRWAGEGSGSVSLALHLQPLRSAAGAAALALTVDLPPASSEARN SAFGFQGRLLHLSAGQRLGVHLHTEARARHAWQLTQGATVLGLF RVTPEGSGNGSGSGMFAQLVAQNVLLIDGPLSWYSDPGLAGVSL TGGLSYKEDTKELVVAKAGVYYVFFQLELRRVVAGEGSGSVSLAL HLQPLRSAAGAAALALTVDLPPASSEARNSAFGFQGRLLHLSAGQ RLGVHLHTEARARHAWQLTQGATVLGLFRVTPEGSGSSSGSCDK THTCPPCPAPELLGGPSVFLFPPKPKDTLMISRTPEVTCVWDVS HEDPEVKFNWYVDGVEVHNAKTKPREEQYSSTYRVVSVLTVLHQ DWLNGKEYKCKVSNKALPAPIEKTISKAKGQPREPQVYTLPPCRD ELTKNQVSLWCLVKGFYPSDIAVEWESNGQPENNYKTTPPVLDS DGSFFLYSKLTVDKSRWQQGNVFSCSVMHEALHNHYTQKSLSLS PGK |
| SEQ-ID:90 | scCD137L-V2-Fc-knob_b | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGNGSGSGMFAQLVAQNVLLID GPLSWYSDPGLAGVSLTGGLSYKEDTKELVVAKAGVYYVFFQLEL RRWAGEGSGSVSLALHLQPLRSAAGAAALALTVDLPPASSEARN SAFGFQGRLLHLSAGQRLGVHLHTEARARHAWQLTQGATVLGLF RVTPEGSGNGSGSGMFAQLVAQNVLLIDGPLSWYSDPGLAGVSL TGGLSYKEDTKELVVAKAGVYYVFFQLELRRVVAGEGSGSVSLAL HLQPLRSAAGAAALALTVDLPPASSEARNSAFGFQGRLLHLSAGQ RLGVHLHTEARARHAWQLTQGATVLGLFRVTPEGSGSDKTHTCP PCPAPELLGGPSVFLFPPKPKDTLMISRTPEVTCVWDVSHEDPE VKFNWYVDGVEVHNAKTKPREEQYSSTYRVVSVLTVLHQDWLN GKEYKCKVSNKALPAPIEKTISKAKGQPREPQVYTLPPCRDELTK NQVSLWCLVKGFYPSDIAVEWESNGQPENNYKTTPPVLDSDGSF FLYSKLTVDKSRWQQGNVFSCSVMHEALHNHYTQKSLSLSPGK |
| SEQ-ID:91 | scCD137L-V2-Fc-hole_a | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGNGSGSGMFAQLVAQNVLLID GPLSWYSDPGLAGVSLTGGLSYKEDTKELVVAKAGVYYVFFQLEL RRWAGEGSGSVSLALHLQPLRSAAGAAALALTVDLPPASSEARN SAFGFQGRLLHLSAGQRLGVHLHTEARARHAWQLTQGATVLGLF RVTPEGSGNGSGSGMFAQLVAQNVLLIDGPLSWYSDPGLAGVSL TGGLSYKEDTKELVVAKAGVYYVFFQLELRRVVAGEGSGSVSLAL HLQPLRSAAGAAALALTVDLPPASSEARNSAFGFQGRLLHLSAGQ RLGVHLHTEARARHAWQLTQGATVLGLFRVTPEGSGSSSGSCDK THTCPPCPAPELLGGPSVFLFPPKPKDTLMISRTPEVTCVWDVS HEDPEVKFNWYVDGVEVHNAKTKPREEQYSSTYRVVSVLTVLHQ DWLNGKEYKCKVSNKALPAPIEKTISKAKGQPREPQVCTLPPSRD ELTKNQVSLSCAVKGFYPSDIAVEWESNGQPENNYKTTPPVLDS DGSFFLVSKLTVDKSRWQQGNVFSCSVMHEALHNHYTQKSLSLS PGK |
| SEQ-ID:92 | scCD137L-V2-Fc-hole_b | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGNGSGSGMFAQLVAQNVLLID GPLSWYSDPGLAGVSLTGGLSYKEDTKELVVAKAGVYYVFFQLEL RRWAGEGSGSVSLALHLQPLRSAAGAAALALTVDLPPASSEARN SAFGFQGRLLHLSAGQRLGVHLHTEARARHAWQLTQGATVLGLF RVTPEGSGNGSGSGMFAQLVAQNVLLIDGPLSWYSDPGLAGVSL TGGLSYKEDTKELVVAKAGVYYVFFQLELRRVVAGEGSGSVSLAL HLQPLRSAAGAAALALTVDLPPASSEARNSAFGFQGRLLHLSAGQ RLGVHLHTEARARHAWQLTQGATVLGLFRVTPEGSGSDKTHTCP PCPAPELLGGPSVFLFPPKPKDTLMISRTPEVTCVWDVSHEDPE VKFNWYVDGVEVHNAKTKPREEQYSSTYRVVSVLTVLHQDWLN GKEYKCKVSNKALPAPIEKTISKAKGQPREPQVCTLPPSRDELTK NQVSLSCAVKGFYPSDIAVEWESNGQPENNYKTTPPVLDSDGSF FLVSKLTVDKSRWQQGNVFSCSVMHEALHNHYTQKSLSLSPGK |
| SEQ-ID:93 | scCD137L-V3-Fc-knob_a | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGSGMFAQLVAQNVLLIDGPLS WYSDPGLAGVSLTGGLSYKEDTKELVVAKAGVYYVFFQLELRRV VAGEGSGSVSLALHLQPLRSAAGAAALALTVDLPPASSEARNSAF GFQGRLLHLSAGQRLGVHLHTEARARHAWQLTQGATVLGLFRVT PEGSGSGMFAQLVAQNVLLIDGPLSWYSDPGLAGVSLTGGLSYK EDTKELVVAKAGVYYVFFQLELRRVVAGEGSGSVSLALHLQPLRS AAGAAALALTVDLPPASSEARNSAFGFQGRLLHLSAGQRLGVHLH TEARARHAWQLTQGATVLGLFRVTPEGSGSSSGSCDKTHTCPPC PAPELLGGPSVFLFPPKPKDTLMISRTPEVTCVWDVSHEDPEVK FNWYVDGVEVHNAKTKPREEQYSSTYRVVSVLTVLHQDWLNGK EYKCKVSNKALPAPIEKTISKAKGQPREPQVYTLPPCRDELTKNQ VSLWCLVKGFYPSDIAVEWESNGQPENNYKTTPPVLDSDGSFFL YSKLTVDKSRWQQGNVFSCSVMHEALHNHYTQKSLSLSPGK |
| SEQ-ID:94 | scCD137L-V3-Fc-knob_b | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGSGMFAQLVAQNVLLIDGPLS WYSDPGLAGVSLTGGLSYKEDTKELVVAKAGVYYVFFQLELRRV VAGEGSGSVSLALHLQPLRSAAGAAALALTVDLPPASSEARNSAF GFQGRLLHLSAGQRLGVHLHTEARARHAWQLTQGATVLGLFRVT PEGSGSGMFAQLVAQNVLLIDGPLSWYSDPGLAGVSLTGGLSYK EDTKELVVAKAGVYYVFFQLELRRVVAGEGSGSVSLALHLQPLRS AAGAAALALTVDLPPASSEARNSAFGFQGRLLHLSAGQRLGVHLH TEARARHAWQLTQGATVLGLFRVTPEGSGSDKTHTCPPCPAPEL LGGPSVFLFPPKPKDTLMISRTPEVTCVVVDVSHEDPEVKFNWYV DGVEVHNAKTKPREEQYSSTYRVVSVLTVLHQDWLNGKEYKCKV SNKALPAPIEKTISKAKGQPREPQVYTLPPCRDELTKNQVSLWCL VKGFYPSDIAVEWESNGQPENNYKTTPPVLDSDGSFFLYSKLTVD KSRWQQGNVFSCSVMHEALHNHYTQKSLSLSPGK |
| SEQ-ID:95 | scCD137L-V3-Fc-hole_a | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGSGMFAQLVAQNVLLIDGPLS WYSDPGLAGVSLTGGLSYKEDTKELVVAKAGVYYVFFQLELRRV VAGEGSGSVSLALHLQPLRSAAGAAALALTVDLPPASSEARNSAF GFQGRLLHLSAGQRLGVHLHTEARARHAWQLTQGATVLGLFRVT PEGSGSGMFAQLVAQNVLLIDGPLSWYSDPGLAGVSLTGGLSYK EDTKELVVAKAGVYYVFFQLELRRVVAGEGSGSVSLALHLQPLRS AAGAAALALTVDLPPASSEARNSAFGFQGRLLHLSAGQRLGVHLH TEARARHAWQLTQGATVLGLFRVTPEGSGSSSGSCDKTHTCPPC PAPELLGGPSVFLFPPKPKDTLMISRTPEVTCVWDVSHEDPEVK FNWYVDGVEVHNAKTKPREEQYSSTYRVVSVLTVLHQDWLNGK EYKCKVSNKALPAPIEKTISKAKGQPREPQVCTLPPSRDELTKNQ VSLSCAVKGFYPSDIAVEWESNGQPENNYKTTPPVLDSDGSFFLV SKLTVDKSRWQQGNVFSCSVMHEALHNHYTQKSLSLSPGK |
| SEQ-ID:96 | scCD137L-V3-Fc-hole_b | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGSGMFAQLVAQNVLLIDGPLS WYSDPGLAGVSLTGGLSYKEDTKELVVAKAGVYYVFFQLELRRV VAGEGSGSVSLALHLQPLRSAAGAAALALTVDLPPASSEARNSAF GFQGRLLHLSAGQRLGVHLHTEARARHAWQLTQGATVLGLFRVT PEGSGSGMFAQLVAQNVLLIDGPLSWYSDPGLAGVSLTGGLSYK EDTKELVVAKAGVYYVFFQLELRRVVAGEGSGSVSLALHLQPLRS AAGAAALALTVDLPPASSEARNSAFGFQGRLLHLSAGQRLGVHLH TEARARHAWQLTQGATVLGLFRVTPEGSGSDKTHTCPPCPAPEL LGGPSVFLFPPKPKDTLMISRTPEVTCVVVDVSHEDPEVKFNWYV DGVEVHNAKTKPREEQYSSTYRVVSVLTVLHQDWLNGKEYKCKV SNKALPAPIEKTISKAKGQPREPQVCTLPPSRDELTKNQVSLSCAV KGFYPSDIAVEWESNGQPENNYKTTPPVLDSDGSFFLVSKLTVDK SRWQQGNVFSCSVMHEALHNHYTQKSLSLSPGK |
| SEQ-ID:97 | scLIGHT-Fc-knob_a | EVNPAAHLTGANSSLTGSGGPLLWETQLGLAFLRGLSYHDGALV VTKAGYYYIYSKVQLGGVGCPLGLASTITHGLYKRTPRYPEELELL VSQQSPCGRATSSSRVWWDSSFLGGWHLEAGEEVWRVLDER LVRLRDGTRSYFGAFMVGSGSGNGSNPAAHLTGANSSLTGSGG PLLWETQLGLAFLRGLSYHDGALVVTKAGYYYIYSKVQLGGVGCP LGLASTITHGLYKRTPRYPEELELLVSQQSPCGRATSSSRVWWD SSFLGGVVHLEAGEEVVVRVLDERLVRLRDGTRSYFGAFMVGSG SGNGSNPAAHLTGANSSLTGSGGPLLWETQLGLAFLRGLSYHDG ALVVTKAGYYYIYSKVQLGGVGCPLGLASTITHGLYKRTPRYPEEL ELLVSQQSPCGRATSSSRVWWDSSFLGGWHLEAGEEVWRVL DERLVRLRDGTRSYFGAFMVGSGSSSGSCDKTHTCPPCPAPELL GGPSVFLFPPKPKDTLMISRTPEVTCVVVDVSHEDPEVKFNWYVD GVEVHNAKTKPREEQYSSTYRVVSVLTVLHQDWLNGKEYKCKVS NKALPAPIEKTISKAKGQPREPQVYTLPPCRDELTKNQVSLWCLV KGFYPSDIAVEWESNGQPENNYKTTPPVLDSDGSFFLYSKLTVDK SRWQQGNVFSCSVMHEALHNHYTQKSLSLSPGK |
| SEQ-ID:98 | scLIGHT-Fc-knob_b | EVNPAAHLTGANSSLTGSGGPLLWETQLGLAFLRGLSYHDGALV VTKAGYYYIYSKVQLGGVGCPLGLASTITHGLYKRTPRYPEELELL VSQQSPCGRATSSSRVWWDSSFLGGWHLEAGEEVWRVLDER LVRLRDGTRSYFGAFMVGSGSGNGSNPAAHLTGANSSLTGSGG PLLWETQLGLAFLRGLSYHDGALVVTKAGYYYIYSKVQLGGVGCP LGLASTITHGLYKRTPRYPEELELLVSQQSPCGRATSSSRVWWD SSFLGGVVHLEAGEEVVVRVLDERLVRLRDGTRSYFGAFMVGSG SGNGSNPAAHLTGANSSLTGSGGPLLWETQLGLAFLRGLSYHDG ALVVTKAGYYYIYSKVQLGGVGCPLGLASTITHGLYKRTPRYPEEL ELLVSQQSPCGRATSSSRVWWDSSFLGGWHLEAGEEVWRVL DERLVRLRDGTRSYFGAFMVGSGSDKTHTCPPCPAPELLGGPSV FLFPPKPKDTLMISRTPEVTCVVVDVSHEDPEVKFNWYVDGVEVH NAKTKPREEQYSSTYRVVSVLTVLHQDWLNGKEYKCKVSNKALP APIEKTISKAKGQPREPQVYTLPPCRDELTKNQVSLWCLVKGFYP SDIAVEWESNGQPENNYKTTPPVLDSDGSFFLYSKLTVDKSRWQ QGNVFSCSVMHEALHNHYTQKSLSLSPGK |
| SEQ-ID:99 | scLIGHT-Fc-hole_a | EVNPAAHLTGANSSLTGSGGPLLWETQLGLAFLRGLSYHDGALV VTKAGYYYIYSKVQLGGVGCPLGLASTITHGLYKRTPRYPEELELL VSQQSPCGRATSSSRVWWDSSFLGGWHLEAGEEVWRVLDER LVRLRDGTRSYFGAFMVGSGSGNGSNPAAHLTGANSSLTGSGG PLLWETQLGLAFLRGLSYHDGALVVTKAGYYYIYSKVQLGGVGCP LGLASTITHGLYKRTPRYPEELELLVSQQSPCGRATSSSRVWWD SSFLGGVVHLEAGEEVVVRVLDERLVRLRDGTRSYFGAFMVGSG SGNGSNPAAHLTGANSSLTGSGGPLLWETQLGLAFLRGLSYHDG ALVVTKAGYYYIYSKVQLGGVGCPLGLASTITHGLYKRTPRYPEEL ELLVSQQSPCGRATSSSRVWWDSSFLGGWHLEAGEEVWRVL DERLVRLRDGTRSYFGAFMVGSGSSSGSCDKTHTCPPCPAPELL GGPSVFLFPPKPKDTLMISRTPEVTCVVVDVSHEDPEVKFNWYVD GVEVHNAKTKPREEQYSSTYRVVSVLTVLHQDWLNGKEYKCKVS NKALPAPIEKTISKAKGQPREPQVCTLPPSRDELTKNQVSLSCAVK GFYPSDIAVEWESNGQPENNYKTTPPVLDSDGSFFLVSKLTVDKS RWQQGNVFSCSVMHEALHNHYTQKSLSLSPGK |
| SEQ-ID:100 | scLIGHT-Fc-hole_b | EVNPAAHLTGANSSLTGSGGPLLWETQLGLAFLRGLSYHDGALV VTKAGYYYIYSKVQLGGVGCPLGLASTITHGLYKRTPRYPEELELL VSQQSPCGRATSSSRVWWDSSFLGGWHLEAGEEVWRVLDER LVRLRDGTRSYFGAFMVGSGSGNGSNPAAHLTGANSSLTGSGG PLLWETQLGLAFLRGLSYHDGALVVTKAGYYYIYSKVQLGGVGCP LGLASTITHGLYKRTPRYPEELELLVSQQSPCGRATSSSRVWWD SSFLGGVVHLEAGEEVVVRVLDERLVRLRDGTRSYFGAFMVGSG SGNGSNPAAHLTGANSSLTGSGGPLLWETQLGLAFLRGLSYHDG ALVVTKAGYYYIYSKVQLGGVGCPLGLASTITHGLYKRTPRYPEEL ELLVSQQSPCGRATSSSRVWWDSSFLGGWHLEAGEEVWRVL DERLVRLRDGTRSYFGAFMVGSGSDKTHTCPPCPAPELLGGPSV FLFPPKPKDTLMISRTPEVTCVVVDVSHEDPEVKFNWYVDGVEVH NAKTKPREEQYSSTYRVVSVLTVLHQDWLNGKEYKCKVSNKALP APIEKTISKAKGQPREPQVCTLPPSRDELTKNQVSLSCAVKGFYP SDIAVEWESNGQPENNYKTTPPVLDSDGSFFLVSKLTVDKSRWQ QGNVFSCSVMHEALHNHYTQKSLSLSPGK |
| SEQ-ID:44 | aCD95L-HC-knob | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSSASTKGPSV FPLAPSSKSTSGGTAALGCLVKDYFPEPVTVSWNSGALTSGVHTF PAVLQSSGLYSLSSVVTVPSSSLGTQTYICNVNHKPSNTKVDKKV EPKSCDKTHTCPPCPAPELLGGPSVFLFPPKPKDTLMISRTPEVT CVVVDVSHEDPEVKFNWYVDGVEVHNAKTKPREEQYSSTYRVV SVLTVLHQDWLNGKEYKCKVSNKALPAPIEKTISKAKGQPREPQV YTLPPCRDELTKNQVSLWCLVKGFYPSDIAVEWESNGQPENNYK TTPPVLDSDGSFFLYSKLTVDKSRWQQGNVFSCSVMHEALHNHY TQKSLSLSPGK |
| SEQ-ID:45 | aCD95L-HC-hole | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSSASTKGPSV FPLAPSSKSTSGGTAALGCLVKDYFPEPVTVSWNSGALTSGVHTF PAVLQSSGLYSLSSVVTVPSSSLGTQTYICNVNHKPSNTKVDKKV EPKSCDKTHTCPPCPAPELLGGPSVFLFPPKPKDTLMISRTPEVT CVVVDVSHEDPEVKFNWYVDGVEVHNAKTKPREEQYSSTYRVV SVLTVLHQDWLNGKEYKCKVSNKALPAPIEKTISKAKGQPREPQV CTLPPSRDELTKNQVSLSCAVKGFYPSDIAVEWESNGQPENNYK TTPPVLDSDGSFFLVSKLTVDKSRWQQGNVFSCSVMHEALHNHY TQKSLSLSPGK |
| SEQ-ID:46 | aCD95L-HC-RF-hole | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSSASTKGPSV FPLAPSSKSTSGGTAALGCLVKDYFPEPVTVSWNSGALTSGVHTF PAVLQSSGLYSLSSVVTVPSSSLGTQTYICNVNHKPSNTKVDKKV EPKSCDKTHTCPPCPAPELLGGPSVFLFPPKPKDTLMISRTPEVT CVVVDVSHEDPEVKFNWYVDGVEVHNAKTKPREEQYSSTYRVV SVLTVLHQDWLNGKEYKCKVSNKALPAPIEKTISKAKGQPREPQV CTLPPSRDELTKNQVSLSCAVKGFYPSDIAVEWESNGQPENNYK TTPPVLDSDGSFFLVSKLTVDKSRWQQGNVFSCSVMHEALHNRF TQKSLSLSPGK |
| SEQ-ID:47 | aCD95L-LC | DIQMTQSPSSLSASVGDRVTITCKASQSIRTSLVWYQQKPGKAPK LLIYKASDLPSGVPSRFSGSGSGTDFTLTISSLQPEDFATYYCQSY DFRDTINNGHSFGQGTKVEIKRTVAAPSVFIFPPSDEQLKSGTASV VCLLNNFYPREAKVQWKVDNALQSGNSQESVTEQDSKDSTYSLS STLTLSKADYEKHKVYACEVTHQGLSSPVTKSFNRGEC |
| SEQ-ID:48 | aCEA-HC-knob | EVQLLESGGGLVQPGGSLRLSCATSGFTFTDYYMNWVRQAPGK GLEWLGFIGNKANGYTTEYSASVKGRFTISRDKSKSTLYLQMNTL QAEDSAIYYCTRDRGLRFYFDYWGQGTLVTVSSASTKGPSVFPL APSSKSTSGGTAALGCLVKDYFPEPVTVSWNSGALTSGVHTFPA VLQSSGLYSLSSVVTVPSSSLGTQTYICNVNHKPSNTKVDKKVEP KSCDKTHTCPPCPAPELLGGPSVFLFPPKPKDTLMISRTPEVTCV VVDVSHEDPEVKFNWYVDGVEVHNAKTKPREEQYSSTYRVVSVL TVLHQDWLNGKEYKCKVSNKALPAPIEKTISKAKGQPREPQVYTL PPCRDELTKNQVSLWCLVKGFYPSDIAVEWESNGQPENNYKTTP PVLDSDGSFFLYSKLTVDKSRWQQGNVFSCSVMHEALHNHYTQK SLSLSPGK |
| SEQ-ID:49 | aCEA-HC-hole | EVQLLESGGGLVQPGGSLRLSCATSGFTFTDYYMNWVRQAPGK GLEWLGFIGNKANGYTTEYSASVKGRFTISRDKSKSTLYLQMNTL QAEDSAIYYCTRDRGLRFYFDYWGQGTLVTVSSASTKGPSVFPL APSSKSTSGGTAALGCLVKDYFPEPVTVSWNSGALTSGVHTFPA VLQSSGLYSLSSVVTVPSSSLGTQTYICNVNHKPSNTKVDKKVEP KSCDKTHTCPPCPAPELLGGPSVFLFPPKPKDTLMISRTPEVTCV VVDVSHEDPEVKFNWYVDGVEVHNAKTKPREEQYSSTYRVVSVL TVLHQDWLNGKEYKCKVSNKALPAPIEKTISKAKGQPREPQVCTL PPSRDELTKNQVSLSCAVKGFYPSDIAVEWESNGQPENNYKTTP PVLDSDGSFFLVSKLTVDKSRWQQGNVFSCSVMHEALHNHYTQK SLSLSPGK |
| SEQ-ID:50 | aCEA-HC-RF-hole | EVQLLESGGGLVQPGGSLRLSCATSGFTFTDYYMNWVRQAPGK GLEWLGFIGNKANGYTTEYSASVKGRFTISRDKSKSTLYLQMNTL QAEDSAIYYCTRDRGLRFYFDYWGQGTLVTVSSASTKGPSVFPL APSSKSTSGGTAALGCLVKDYFPEPVTVSWNSGALTSGVHTFPA VLQSSGLYSLSSVVTVPSSSLGTQTYICNVNHKPSNTKVDKKVEP KSCDKTHTCPPCPAPELLGGPSVFLFPPKPKDTLMISRTPEVTCV VVDVSHEDPEVKFNWYVDGVEVHNAKTKPREEQYSSTYRVVSVL TVLHQDWLNGKEYKCKVSNKALPAPIEKTISKAKGQPREPQVCTL PPSRDELTKNQVSLSCAVKGFYPSDIAVEWESNGQPENNYKTTP PVLDSDGSFFLVSKLTVDKSRWQQGNVFSCSVMHEALHNRFTQK SLSLSPGK |
| SEQ-ID:51 | aCEA-LC | QTVLTQSPSSLSVSVGDRVTITCRASSSVTYIHWYQQKPGLAPKS LIYATSNLASGVPSRFSGSGSGTDYTFTISSLQPEDIATYYCQHWS SKPPTFGQGTKVEVKRTVAAPSVFIFPPSDEQLKSGTASVVCLLN NFYPREAKVQWKVDNALQSGNSQESVTEQDSKDSTYSLSSTLTL SKADYEKHKVYACEVTHQGLSSPVTKSFNRGEC |
| SEQ-ID:52 | aPD-L1-HC-knob | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSSASTKGPSVFPLAPS SKSTSGGTAALGCLVKDYFPEPVTVSWNSGALTSGVHTFPAVLQ SSGLYSLSSVVTVPSSSLGTQTYICNVNHKPSNTKVDKKVEPKSC DKTHTCPPCPAPELLGGPSVFLFPPKPKDTLMISRTPEVTCVVVD VSHEDPEVKFNWYVDGVEVHNAKTKPREEQYSSTYRVVSVLTVL HQDWLNGKEYKCKVSNKALPAPIEKTISKAKGQPREPQVYTLPPC RDELTKNQVSLWCLVKGFYPSDIAVEWESNGQPENNYKTTPPVL DSDGSFFLYSKLTVDKSRWQQGNVFSCSVMHEALHNHYTQKSLS |
| | | LSPGK |
| SEQ-ID:53 | aPD-L1-HC-hole | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSSASTKGPSVFPLAPS SKSTSGGTAALGCLVKDYFPEPVTVSWNSGALTSGVHTFPAVLQ SSGLYSLSSVVTVPSSSLGTQTYICNVNHKPSNTKVDKKVEPKSC DKTHTCPPCPAPELLGGPSVFLFPPKPKDTLMISRTPEVTCVVVD VSHEDPEVKFNWYVDGVEVHNAKTKPREEQYSSTYRVVSVLTVL HQDWLNGKEYKCKVSNKALPAPIEKTISKAKGQPREPQVCTLPPS RDELTKNQVSLSCAVKGFYPSDIAVEWESNGQPENNYKTTPPVL DSDGSFFLVSKLTVDKSRWQQGNVFSCSVMHEALHNHYTQKSLS LSPGK |
| SEQ-ID:54 | aPD-L1-HC-RF-hole | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSSASTKGPSVFPLAPS SKSTSGGTAALGCLVKDYFPEPVTVSWNSGALTSGVHTFPAVLQ SSGLYSLSSVVTVPSSSLGTQTYICNVNHKPSNTKVDKKVEPKSC DKTHTCPPCPAPELLGGPSVFLFPPKPKDTLMISRTPEVTCVVVD VSHEDPEVKFNWYVDGVEVHNAKTKPREEQYSSTYRVVSVLTVL HQDWLNGKEYKCKVSNKALPAPIEKTISKAKGQPREPQVCTLPPS RDELTKNQVSLSCAVKGFYPSDIAVEWESNGQPENNYKTTPPVL DSDGSFFLVSKLTVDKSRWQQGNVFSCSVMHEALHNRFTQKSLS LSPGK |
| SEQ-ID:55 | aPD-L1-LC | QSALTQPASVSGSPGQSITISCTGTSSDVGGYNYVSWYQQHPGK APKLMIYDVSNRPSGVSNRFSGSKSGNTASLTISGLQAEDEADYY CSSYTSSSTRVFGTGTKVEIKRTVAAPSVFIFPPSDEQLKSGTASV VCLLNNFYPREAKVQWKVDNALQSGNSQESVTEQDSKDSTYSLS STLTLSKADYEKHKVYACEVTHQGLSSPVTKSFNRGEC |
| SEQ-ID:56 | aCEA-hc-scCD40L-RBD | EVQLLESGGGLVQPGGSLRLSCATSGFTFTDYYMNWVRQAPGK GLEWLGFIGNKANGYTTEYSASVKGRFTISRDKSKSTLYLQMNTL QAEDSAIYYCTRDRGLRFYFDYWGQGTLVTVSSASTKGPSVFPF APSSKSTSGGTAALGCLVKDYFPEPVTVSWNSGALTSGVHTFPA VLQSSGLYSLSSVVTVPSSSLGTQTYICNVNHKPSNTKVDKKVEP KSCDKTHGSGSSSSSSQIAAHVISEASSKTTSVLQWAEKGYYTMS NNLVTLENGKQLTVKRQGLYYIYAQVTFCSNREASSQAPFIASLSL KSPGRFERILLRAANTHSSAKPCGQQSIHLGGVFELQPGASVFVN VTDPSQVSHGTGFTSFGLLKLGSGSGNGSQIAAHVISEASSKTTS VLQWAEKGYYTMSNNLVTLENGKQLTVKRQGLYYIYAQVTFCSN REASSQAPFIASLSLKSPGRFERILLRAANTHSSAKPCGQQSIHLG GVFELQPGASVFVNVTDPSQVSHGTGFTSFGLLKLGSGSGNGSQ IAAHVISEASSKTTSVLQWAEKGYYTMSNNLVTLENGKQLTVKRQ GLYYIYAQVTFCSNREASSQAPFIASLSLKSPGRFERILLRAANTH SSAKPCGQQSIHLGGVFELQPGASVFVNVTDPSQVSHGTGFTSF GLLKL |
| SEQ-ID:57 | aCD95L-hc-scCD40L-RBD | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSSASTKGPSV FPLAPSSKSTSGGTAALGCLVKDYFPEPVTVSWNSGALTSGVHTF PAVLQSSGLYSLSSVVTVPSSSLGTQTYICNVNHKPSNTKVDKKV EPKSCDKTHGSGSSSSSSQIAAHVISEASSKTTSVLQWAEKGYYT MSNNLVTLENGKQLTVKRQGLYYIYAQVTFCSNREASSQAPFIAS LSLKSPGRFERILLRAANTHSSAKPCGQQSIHLGGVFELQPGASV FVNVTDPSQVSHGTGFTSFGLLKLGSGSGNGSQIAAHVISEASSK TTSVLQWAEKGYYTMSNNLVTLENGKQLTVKRQGLYYIYAQVTFC SNREASSQAPFIASLSLKSPGRFERILLRAANTHSSAKPCGQQSIH LGGVFELQPGASVFVNVTDPSQVSHGTGFTSFGLLKLGSGSGNG SQIAAHVISEASSKTTSVLQWAEKGYYTMSNNLVTLENGKQLTVK RQGLYYIYAQVTFCSNREASSQAPFIASLSLKSPGRFERILLRAAN THSSAKPCGQQSIHLGGVFELQPGASVFVNVTDPSQVSHGTGFT SFGLLKL |
| SEQ-ID:58 | aPDL1-hc-scCD40L-RBD | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSSASTKGPSVFPLAPS SKSTSGGTAALGCLVKDYFPEPVTVSWNSGALTSGVHTFPAVLQ SSGLYSLSSVVTVPSSSLGTQTYICNVNHKPSNTKVDKKVEPKSC DKTHGSGSSSSSSQIAAHVISEASSKTTSVLQWAEKGYYTMSNNL VTLENGKQLTVKRQGLYYIYAQVTFCSNREASSQAPFIASLSLKSP GRFERILLRAANTHSSAKPCGQQSIHLGGVFELQPGASVFVNVTD PSQVSHGTGFTSFGLLKLGSGSGNGSQIAAHVISEASSKTTSVLQ WAEKGYYTMSNNLVTLENGKQLTVKRQGLYYIYAQVTFCSNREA SSQAPFIASLSLKSPGRFERILLRAANTHSSAKPCGQQSIHLGGVF ELQPGASVFVNVTDPSQVSHGTGFTSFGLLKLGSGSGNGSQIAA HVISEASSKTTSVLQWAEKGYYTMSNNLVTLENGKQLTVKRQGLY YIYAQVTFCSNREASSQAPFIASLSLKSPGRFERILLRAANTHSSA KPCGQQSIHLGGVFELQPGASVFVNVTDPSQVSHGTGFTSFGLL KL |
| SEQ-ID:101 | aCD95L-hc-scCD27L-RBD | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSSASTKGPSV FPLAPSSKSTSGGTAALGCLVKDYFPEPVTVSWNSGALTSGVHTF PAVLQSSGLYSLSSVVTVPSSSLGTQTYICNVNHKPSNTKVDKKV EPKSCDKTHGSGSSSSSSESLGWDVAELQLNHTGPQQDPRLYW QGGPALGRSFLHGPELDKGQLRIHRDGIYMVHIQVTLAICSSTTAS RHHPTTLAVGICSPASRSISLLRLSFHQGCTIASQRLTPLARGDTL CTNLTGTLLPSRNTDETFFGVQWVRPGSGSGNGSESLGWDVAE LQLNHTGPQQDPRLYWQGGPALGRSFLHGPELDKGQLRIHRDGI YMVHIQVTLAICSSTTASRHHPTTLAVGICSPASRSISLLRLSFHQG CTIASQRLTPLARGDTLCTNLTGTLLPSRNTDETFFGVQWVRPGS GSGNGSESLGWDVAELQLNHTGPQQDPRLYWQGGPALGRSFL HGPELDKGQLRIHRDGIYMVHIQVTLAICSSTTASRHHPTTLAVGIC SPASRSISLLRLSFHQGCTIASQRLTPLARGDTLCTNLTGTLLPSR NTDETFFGVQWVRP |
| SEQ-ID:102 | aPDL1-hc-scCD27L-RBD | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSSASTKGPSVFPLAPS SKSTSGGTAALGCLVKDYFPEPVTVSWNSGALTSGVHTFPAVLQ SSGLYSLSSVVTVPSSSLGTQTYICNVNHKPSNTKVDKKVEPKSC DKTHGSGSSSSSSESLGWDVAELQLNHTGPQQDPRLYWQGGPA LGRSFLHGPELDKGQLRIHRDGIYMVHIQVTLAICSSTTASRHHPT TLAVGICSPASRSISLLRLSFHQGCTIASQRLTPLARGDTLCTNLTG TLLPSRNTDETFFGVQWVRPGSGSGNGSESLGWDVAELQLNHT GPQQDPRLYWQGGPALGRSFLHGPELDKGQLRIHRDGIYMVHIQ VTLAICSSTTASRHHPTTLAVGICSPASRSISLLRLSFHQGCTIASQ RLTPLARGDTLCTNLTGTLLPSRNTDETFFGVQWVRPGSGSGNG SESLGWDVAELQLNHTGPQQDPRLYWQGGPALGRSFLHGPELD KGQLRIHRDGIYMVHIQVTLAICSSTTASRHHPTTLAVGICSPASRS ISLLRLSFHQGCTIASQRLTPLARGDTLCTNLTGTLLPSRNTDETFF GVQWVRP |
| SEQ-ID:103 | aCD95L-hc-scGITRL-RBD | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSSASTKGPSV FPLAPSSKSTSGGTAALGCLVKDYFPEPVTVSWNSGALTSGVHTF PAVLQSSGLYSLSSVVTVPSSSLGTQTYICNVNHKPSNTKVDKKV EPKSCDKTHGSGSSSSSSQPCMAKFGPLPSKWQMASSEPPCVN KVSDWKLEILQNGLYLIYGQVAPNANYNDVAPFEVRLYKNKDMIQ TLTNKSKIQNVGGTYELHVGDTIDLIFNSEHQVLKNNTYWGIILLAN PQFISGSGSGNGSEPCMAKFGPLPSKWQMASSEPPCVNKVSDW KLEILQNGLYLIYGQVAPNANYNDVAPFEVRLYKNKDMIQTLTNKS KIQNVGGTYELHVGDTIDLIFNSEHQVLKNNTYWGIILLANPQFISG SGSGNGSEPCMAKFGPLPSKWQMASSEPPCVNKVSDWKLEILQ NGLYLIYGQVAPNANYNDVAPFEVRLYKNKDMIQTLTNKSKIQNV GGTYELHVGDTIDLIFNSEHQVLKNNTYWGIILLANPQFIS |
| SEQ-ID:104 | aPDL1-hc-scGITRL-RBD | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSSASTKGPSVFPLAPS SKSTSGGTAALGCLVKDYFPEPVTVSWNSGALTSGVHTFPAVLQ SSGLYSLSSVVTVPSSSLGTQTYICNVNHKPSNTKVDKKVEPKSC DKTHGSGSSSSSSQPCMAKFGPLPSKWQMASSEPPCVNKVSD WKLEILQNGLYLIYGQVAPNANYNDVAPFEVRLYKNKDMIQTLTNK SKIQNVGGTYELHVGDTIDLIFNSEHQVLKNNTYWGIILLANPQFIS GSGSGNGSEPCMAKFGPLPSKWQMASSEPPCVNKVSDWKLEIL QNGLYLIYGQVAPNANYNDVAPFEVRLYKNKDMIQTLTNKSKIQN VGGTYELHVGDTIDLIFNSEHQVLKNNTYWGIILLANPQFISGSGS GNGSEPCMAKFGPLPSKWQMASSEPPCVNKVSDWKLEILQNGL YLIYGQVAPNANYNDVAPFEVRLYKNKDMIQTLTNKSKIQNVGGT YELHVGDTIDLIFNSEHQVLKNNTYWGIILLANPQFIS |
| SEQ-ID:59 | aCD137-VH | QVQLQQWGAGLLKPSETLSLTCAVYGGSFSGYYWSWIRQSPEK GLEWIGEINHGGYVTYNPSLESRVTISVDTSKNQFSLKLSSVTAAD TAVYYCARDYGPGNYDWYFDLWGRGTLVTVSS |
| SEQ-ID:60 | aCD137-VL | EIVLTQSPATLSLSPGERATLSCRASQSVSSYLAWYQQKPGQAPR LLIYDASNRATGIPARFSGSGSGTDFTLTISSLEPEDFAVYYCQQR SNWPPALTFGGGTKVEIKR |
| SEQ-ID:61 | aMeso-VH | EVQLVESGGGLVQPGGSLRLSCAASGYTFTTYWMHWVRQAPGK GLEWVGYIRPSTGYTEYNQKFKDRFTISADTSKNTAYLQMNSLRA EDTAVYYCARSRWLLDYWGQGTLVTVSS |
| SEQ-ID:62 | aMeso-VL | DIQMTQSPSSLSASVGDRVTITCKSSQSVLYSSNQKNYLAWFQQ KPGKAPKLLIYWASTRESGVPSRFSGSGSGTDFTLTISSLQPEDF ATYFCHQYLSSYTFGQGTKVEIKR |
| SEQ-ID:63 | aCD25-VH | QVQLVQSGAEVKKPGSSVKVSCKASGYTFTSYRMHWVRQAPGQ GLEWIGYINPSTGYTEYNQKFKDKATITADESTNTAYMELSSLRSE DTAVYYCARGGGVFDYWGQGTTLTVSS |
| SEQ-ID:64 | aCD25-VL | DIQMTQSPSTLSASVGDRVTITCSASSSISYMHWYQQKPGKAPKL LIYTTSNLASGVPARFSGSGSGTEFTLTISSLQPDDFATYYCHQRS TYPLTFGSGTKVEVKR |
| SEQ-ID:65 | aPD1-a-VH | QVQLVESGGGVVQPGRSLRLDCKASGITFSNSGMHWVRQAPGK GLEWVAVIWYDGSKRYYADSVKGRFTISRDNSKNTLFLQMNSLR AEDTAVYYCATNDDYWGQGTLVTVSS |
| SEQ-ID:66 | aPD1-a-VL | EIVLTQSPATLSLSPGERATLSCRASQSVSSYLAWYQQKPGQAPR LLIYDASNRATGIPARFSGSGSGTDFTLTISSLEPEDFAVYYCQQS SNWPRTFGQGTKVEIKR |
| SEQ-ID:67 | aPD1-b-VH | QVQLVQSGVEVKKPGASVKVSCKASGYTFTNYYMYWVRQAPGQ GLEWMGGINPSNGGTNFNEKFKNRVTLTTDSSTTTAYMELKSLQ FDDTAVYYCARRDYRFDMGFDYWGQGTTVTVSS |
| SEQ-ID:68 | aPD1-b-VL | EIVLTQSPATLSLSPGERATLSCRASKGVSTSGYSYLHWYQQKPG QAPRLLIYLASYLESGVPARFSGSGSGTDFTLTISSLEPEDFAVYY CQHSRDLPLTFGGGTKVEIKR |
| SEQ-ID:113 | aCEA-a-VH | EVQLLESGGGLVQPGGSLRLSCATSGFTFTDYYMNWVRQAPGK GLEWLGFIGNKANGYTTEYSASVKGRFTISRDKSKSTLYLQMNTL QAEDSAIYYCTRDRGLRFYFDYWGQGTLVTVSS |
| SEQ-ID:114 | aCEA-a-VL | QTVLTQSPSSLSVSVGDRVTITCRASSSVTYIHWYQQKPGLAPKS LIYATSNLASGVPSRFSGSGSGTDYTFTISSLQPEDIATYYCQHWS SKPPTFGQGTKVEVKR |
| SEQ-ID:115 | aCEA-b-VH | EVQLVESGGGLVQPGRSLRLSCAASGFTVSSYWMHWVRQAPGK GLEWVGFIRNKANGGTTEYAASVKGRFTISRDDSKNTLYLQMNSL RAEDTAVYYCARDRGLRFYFDYWGQGTTVTVSS |
| SEQ-ID:116 | aCEA-b-VL | QAVLTQPASLSASPGASASLTCTLRRGINVGAYSIYWYQQKPGSP PQYLLRYKSDSDKQQGSGVSSRFSASKDASANAGILLISGLQSED EADYYCMIWHSGASAVFGGGTKLTVL |
| SEQ-ID:117 | aCD95L-VH | EVQLVESGGGLVQPGGSLRLSCAASGFSFSDHYWMCWVRQAP GKGLEWVACIYTADSDSYYADSVKGRFTISKDSSKNTLYLQMNSL RAEDTAVYYCARNGAYAGGPYGDLWGQGTLVTVSS |
| SEQ-ID:118 | aCD95L-VL | DIQMTQSPSSLSASVGDRVTITCKASQSIRTSLVWYQQKPGKAPK LLIYKASDLPSGVPSRFSGSGSGTDFTLTISSLQPEDFATYYCQSY DFRDTINNGHSFGQGTKVEIKR |
| SEQ-ID:69 | scCD40L-RBD | QIAAHVISEASSKTTSVLQWAEKGYYTMSNNLVTLENGKQLTVKR QGLYYIYAQVTFCSNREASSQAPFIASLsLKSPGRFERILLRAANTH SSAKPCGQQSIHLGGVFELQPGASVFVNVTDPSQVSHGTGFTSF GLLKLGSGSGNGSQIAAHVISEASSKTTSVLQWAEKGYYTMSNNL VTLENGKQLTVKRQGLYYIYAQVTFCSNREASSQAPFIASLsLKSP GRFERILLRAANTHSSAKPCGQQSIHLGGVFELQPGASVFVNVTD PSQVSHGTGFTSFGLLKLGSGSGNGSQIAAHVISEASSKTTSVLQ WAEKGYYTMSNNLVTLENGKQLTVKRQGLYYIYAQVTFCSNREA SSQAPFIASLsLKSPGRFERILLRAANTHSSAKPCGQQSIHLGGVF ELQPGASVFVNVTDPSQVSHGTGFTSFGLLKL |
| SEQ-ID:70 | scCD27L-RBD | QSLGWDVAELQLNHTGPQQDPRLYWQGGPALGRSFLHGPELDK GQLRIHRDGIYMVHIQVTLAICSSTTASRHHPTTLAVGICSPASRSI SLLRLSFHQGCTIASQRLTPLARGDTLCTNLTGTLLPSRNTDETFF GVQWVRPGSGSGNGSESLGWDVAELQLNHTGPQQDPRLYWQG GPALGRSFLHGPELDKGQLRIHRDGIYMVHIQVTLAICSSTTASRH HPTTLAVGICSPASRSISLLRLSFHQGCTIASQRLTPLARGDTLCTN LTGTLLPSRNTDETFFGVQWVRPGSGSGNGSESLGWDVAELQL NHTGPQQDPRLYWQGGPALGRSFLHGPELDKGQLRIHRDGIYMV HIQVTLAICSSTTASRHHPTTLAVGICSPASRSISLLRLSFHQGCTIA SQRLTPLARGDTLCTNLTGTLLPSRNTDETFFGVQWVRP |
| SEQ-ID:119 | scCD27L-V2-RBD | DVAELQLDHTGPQQDPRLYWQGGPALGRSFLHGPELDKGQLRIH RDGIYMVHIQVTLAICSSTTASRHHPTTLAVGICSPASRSISLLRLS FHQGCTIASQRLTPLARGDTLCTNLTGTLLPSRNTDETFFGVQWV GSGSDVAELQLDHTGPQQDPRLYWQGGPALGRSFLHGPELDKG QLRIHRDGIYMVHIQVTLAICSSTTASRHHPTTLAVGICSPASRSISL LRLSFHQGCTIASQRLTPLARGDTLCTNLTGTLLPSRNTDETFFGV QWVGSGSDVAELQLDHTGPQQDPRLYWQGGPALGRSFLHGPEL DKGQLRIHRDGIYMVHIQVTLAICSSTTASRHHPTTLAVGICSPASR SISLLRLSFHQGCTIASQRLTPLARGDTLCTNLTGTLLPSRNTDETF FGVQWV |
| SEQ-ID:71 | scGITRL-RBD | QPCMAKFGPLPSKWQMASSEPPCVNKVSDWKLEILQNGLYLIYG QVAPNANYNDVAPFEVRLYKNKDMIQTLTNKSKIQNVGGTYELHV GDTIDLIFNSEHQVLKNNTYWGIILLANPQFISGSGSGNGSEPCMA KFGPLPSKWQMASSEPPCVNKVSDWKLEILQNGLYLIYGQVAPN ANYNDVAPFEVRLYKNKDMIQTLTNKSKIQNVGGTYELHVGDTIDL IFNSEHQVLKNNTYWGIILLANPQFISGSGSGNGSEPCMAKFGPLP SKWQMASSEPPCVNKVSDWKLEILQNGLYLIYGQVAPNANYNDV APFEVRLYKNKDMIQTLTNKSKIQNVGGTYELHVGDTIDLIFNSEH QVLKNNTYWGIILLANPQFIS |
| SEQ-ID:72 | scCD137L-RBD | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVGSGSGNGSQGMFAQLVAQNVLLIDG PLSWYSDPGLAGVSLTGGLSYKEDTKELVVAKAGVYYVFFQLELR RWAGEGSGSVSLALHLQPLRSAAGAAALALTVDLPPASSEARNS AFGFQGRLLHLSAGQRLGVHLHTEARARHAWQLTQGATVLGLFR VGSGSGNGSQGMFAQLVAQNVLLIDGPLSWYSDPGLAGVSLTG GLSYKEDTKELVVAKAGVYYVFFQLELRRVVAGEGSGSVSLALHL QPLRSAAGAAALALTVDLPPASSEARNSAFGFQGRLLHLSAGQRL GVHLHTEARARHAWQLTQGATVLGLFRV |
| SEQ-ID:105 | scCD137L-V2-RBD | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGNGSGSGMFAQLVAQNVLLID GPLSWYSDPGLAGVSLTGGLSYKEDTKELVVAKAGVYYVFFQLEL RRWAGEGSGSVSLALHLQPLRSAAGAAALALTVDLPPASSEARN SAFGFQGRLLHLSAGQRLGVHLHTEARARHAWQLTQGATVLGLF RVTPEGSGNGSGSGMFAQLVAQNVLLIDGPLSWYSDPGLAGVSL TGGLSYKEDTKELVVAKAGVYYVFFQLELRRVVAGEGSGSVSLAL HLQPLRSAAGAAALALTVDLPPASSEARNSAFGFQGRLLHLSAGQ RLGVHLHTEARARHAWQLTQGATVLGLFRVTPE |
| SEQ-ID:106 | scCD137L-V3-RBD | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPEGSGSGMFAQLVAQNVLLIDGPLS WYSDPGLAGVSLTGGLSYKEDTKELVVAKAGVYYVFFQLELRRV VAGEGSGSVSLALHLQPLRSAAGAAALALTVDLPPASSEARNSAF GFQGRLLHLSAGQRLGVHLHTEARARHAWQLTQGATVLGLFRVT PEGSGSGMFAQLVAQNVLLIDGPLSWYSDPGLAGVSLTGGLSYK EDTKELVVAKAGVYYVFFQLELRRVVAGEGSGSVSLALHLQPLRS AAGAAALALTVDLPPASSEARNSAFGFQGRLLHLSAGQRLGVHLH TEARARHAWQLTQGATVLGLFRVTPE |
| SEQ-ID:107 | scCD137L-V4-RBD | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPGSGNGSGSGMFAQLVAQNVLLIDG PLSWYSDPGLAGVSLTGGLSYKEDTKELVVAKAGVYYVFFQLELR RWAGEGSGSVSLALHLQPLRSAAGAAALALTVDLPPASSEARNS AFGFQGRLLHLSAGQRLGVHLHTEARARHAWQLTQGATVLGLFR VTPGSGNGSGSGMFAQLVAQNVLLIDGPLSWYSDPGLAGVSLTG GLSYKEDTKELVVAKAGVYYVFFQLELRRVVAGEGSGSVSLALHL QPLRSAAGAAALALTVDLPPASSEARNSAFGFQGRLLHLSAGQRL GVHLHTEARARHAWQLTQGATVLGLFRVTP |
| SEQ-ID:108 | scCD137L-V5-RBD | QGMFAQLVAQNVLLIDGPLSWYSDPGLAGVSLTGGLSYKEDTKE LVVAKAGVYYVFFQLELRRVVAGEGSGSVSLALHLQPLRSAAGAA ALALTVDLPPASSEARNSAFGFQGRLLHLSAGQRLGVHLHTEARA RHAWQLTQGATVLGLFRVTPGSGSGMFAQLVAQNVLLIDGPLSW YSDPGLAGVSLTGGLSYKEDTKELVVAKAGVYYVFFQLELRRVVA GEGSGSVSLALHLQPLRSAAGAAALALTVDLPPASSEARNSAFGF QGRLLHLSAGQRLGVHLHTEARARHAWQLTQGATVLGLFRVTPG SGSGMFAQLVAQNVLLIDGPLSWYSDPGLAGVSLTGGLSYKEDT KELVVAKAGVYYVFFQLELRRVVAGEGSGSVSLALHLQPLRSAAG AAALALTVDLPPASSEARNSAFGFQGRLLHLSAGQRLGVHLHTEA RARHAWQLTQGATVLGLFRVTP |
| SEQ-ID:73 | scLIGHT-RBD | EVNPAAHLTGANSSLTGSGGPLLWETQLGLAFLRGLSYHDGALV VTKAGYYYIYSKVQLGGVGCPLGLASTITHGLYKRTPRYPEELELL VSQQSPCGRATSSSRVWWDSSFLGGVVHLEAGEEVVVRVLDER LVRLRDGTRSYFGAFMVGSGSGNGSNPAAHLTGANSSLTGSGG PLLWETQLGLAFLRGLSYHDGALVVTKAGYYYIYSKVQLGGVGCP LGLASTITHGLYKRTPRYPEELELLVSQQSPCGRATSSSRVWWD SSFLGGVVHLEAGEEVVVRVLDERLVRLRDGTRSYFGAFMVGSG SGNGSNPAAHLTGANSSLTGSGGPLLWETQLGLAFLRGLSYHDG ALVVTKAGYYYIYSKVQLGGVGCPLGLASTITHGLYKRTPRYPEEL ELLVSQQSPCGRATSSSRVWWDSSFLGGWHLEAGEEVWRVL DERLVRLRDGTRSYFGAFMV |
| SEQ-ID:74 | aPD-L1-VH | EVQLLESGGGLVQPGGSLRLSCAASGFTFSSYIMMWVRQAPGK GLEWVSSIYPSGGITFYADTVKGRFTISRDNSKNTLYLQMNSLRAE DTAVYYCARIKLGTVTTVDYWGQGTLVTVSS |
| SEQ-ID:75 | aPD-L1-VL | QSALTQPASVSGSPGQSITISCTGTSSDVGGYNYVSWYQQHPGK APKLMIYDVSNRPSGVSNRFSGSKSGNTASLTISGLQAEDEADYY CSSYTSSSTRVFGTGTKVEIKR |
| SEQ-ID:76 | Streptag-ll element-1 | SSSSSSAWSHPQFEK |
| SEQ-ID:77 | Streptag-ll element-2 | GSSSSSSAWSHPQFEK |

### Example 4: Targeting Increases Agonistic Activity of SAB Molecules

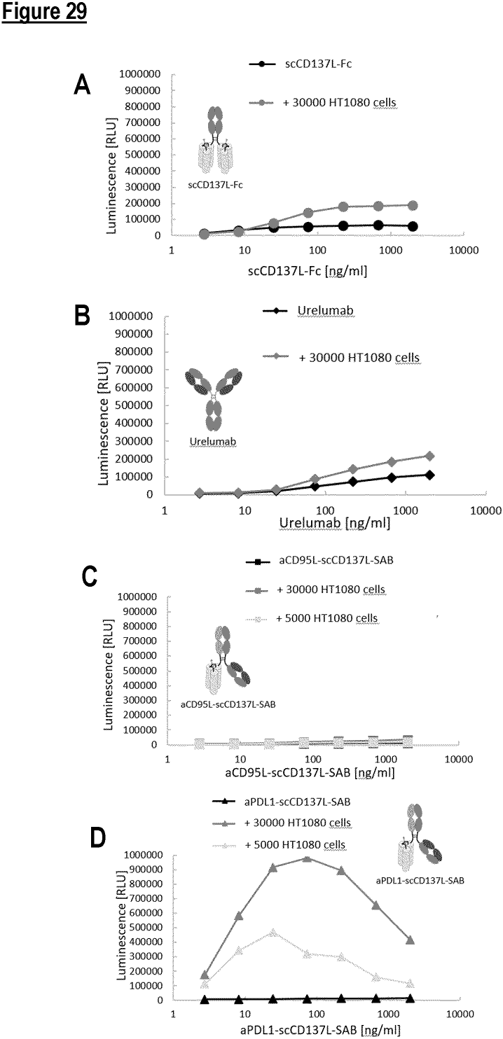

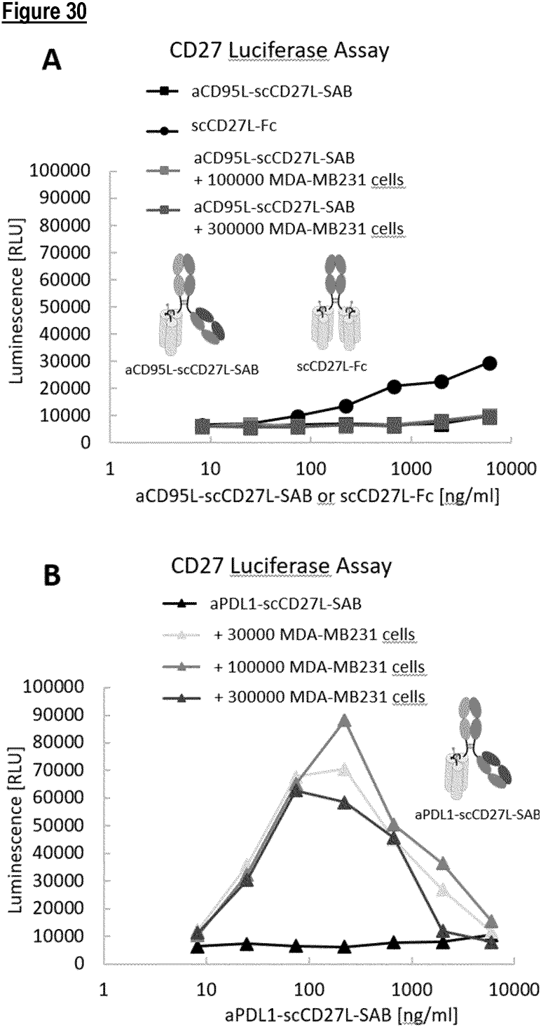

The contribution of the targeting domain for the agonistic activity of the bispecific molecules has been demonstrated. In a CD137 Luciferase assay, the enormous increase in the agonistic activity of aPDL1-scCD137L-SAB by the addition of HT1080 cells is evident (see FIG. 29). Almost every HT1080 cell expresses PD-L1 whereas they are negative for CD95L expression. Therefore it is not surprising that these cells fail to relevantly increase the agonistic activity of aCD95L-scCD137L-SAB (a non-targeting control). However, the massive increase in agonism by the PD-L1 targeting construct aPDL1-scCD137L-SAB (approx. 16-fold higher than the activity observed for the hexavalent scCD137L-Fc) is surprising and underlines the potential of these molecules as potent co-stimulators targeting PD-L1 expressing tumors. This increased agonistic activity of aPDL1-scCD137L-SAB has also been demonstrated by the addition of further PD-L1 expressing cancer cell lines AsPC-1, LN-18 and MDA-MB231 (not shown). The bell-shaped concentration dependency is regularly observed for co-stimulatory agonists. The fact, that in this case the bell-shaped concentration dependency is very pronounced, can be explained by the high degree of agonism: there is an optimum for the number of agonistic molecules crosslinked by PD-L1 expressing cells and a higher number of ligand trimers leads to a decrease of receptors in the ligand/receptor complexes.

Similarly, in a CD27 Luciferase assay, the enormous increase in the agonistic activity of aPDL1-scCD27L-SAB by the addition of MDA-MB231 cells is evident (see FIG. 30). Almost every MDA-MB231 cell expresses PD-L1 whereas they are negative for CD95L expression. Therefore it is not surprising that these cells fail to increase the agonistic activity of aCD95L-scCD27L-SAB (a non-targeting control). However, the massive increase in agonism by the PD-L1 targeting construct aPDL1-scCD27L-SAB (approx. 3-fold higher than the activity observed for the hexavalent scCD27L-Fc) is surprising and underlines the potential of these molecules as potent co-stimulators targeting PD-L1 expressing tumors. This increased agonistic activity of aPDL1-scCD27L-SAB has also been demonstrated by the addition of further PD-L1 expressing cancer cell lines LN-18 and HT1080 (not shown).

### Example 5: Bispecific CD137L Molecules Activate T Cell Proliferation

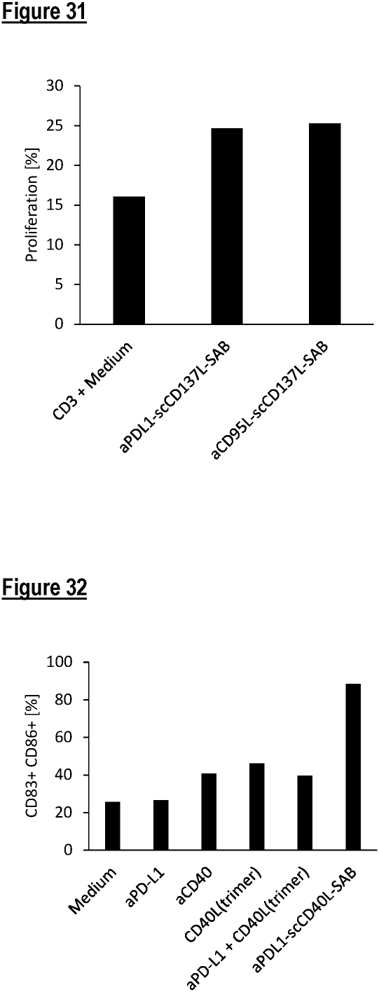

The biological activity of the scCD137L bispecific molecules is demonstrated in FIG. 31 employing a T cell activation assay. The bispecific molecules aPDL1-scCD137L-SAB and aCD95L-scCD137L-SAB lead to a similar proliferation of T cells in the presence of anti-CD3 stimulation.

### Example 6: aPD-L1- scCD40L-SAB Bispecific Shows Excellent Stimulation of Dendritic Cells

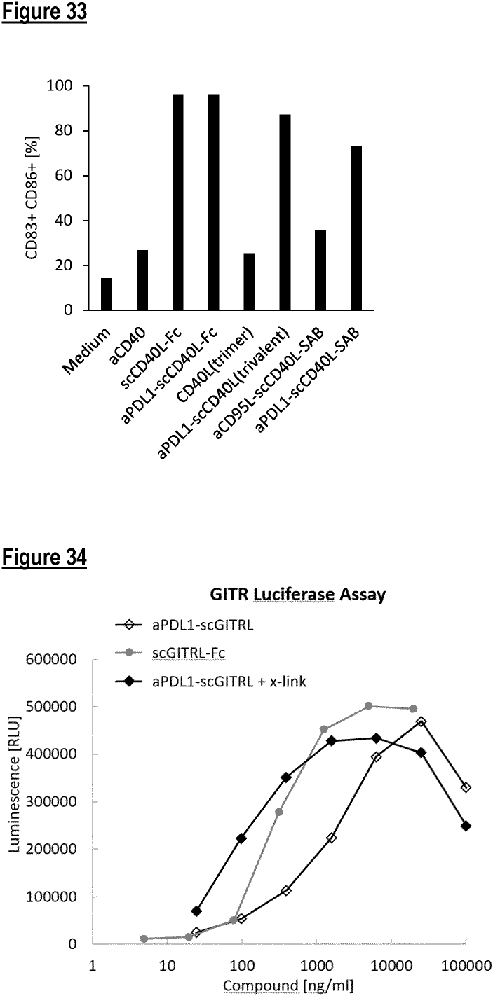

The biological activity of the scCD40L bispecific molecules and further CD40 agonists is demonstrated in FIG. 32 and FIG. 33 employing an immature dendritic cell activation assay. In the experiment displayed in FIG. 32, the bispecific molecule aPDL1-scCD40L-SAB combining trivalent scCD40L with the anti-PD-L1 antibody fragment is the most potent activator of dendritic cells with an activation level (CD86+ / CD83+) of 88.31%. Thus, combining both moieties in one molecule is far more effective than having these moieties added as two separate molecules, i.e. aPD-L1 antibody + CD40L(trimer) which show an activation level of only 39.48 %. aCD40 monoclonal antibody and CD40L(trimer) both show a moderate level of activity (40.59 % and 46.10 %), whereas aPD-L1 monoclonal antibody has the same activity as medium control.

In the experiment displayed in FIG. 33, the bispecific molecules combining trivalent scCD40L with the anti-PD-L1 antibody fragment are very potent activators of dendritic cells with an activation level (CD86+ /CD83+) similar to that of scCD40L-Fc: 73.00% for aPDL1-scCD40L-SAB, 87.18% for aPDL1-scCD40L(trivalent) and 96.12 % for aPDL1-scCD40L-Fc. In contrast, the bispecific molecule aCD95L-scCD40L-SAB which targets CD95L instead of PD-L1 has only a moderate level of activation due to the lack of CD95L expression on monocytes, which do express PD-L1. Low activation levels are also seen for aCD40 monoclonal antibody and CD40L(trimer).

### Example 7: GITR Luciferase Assay

The biological activity of the aPDL1-scGITRL bispecific molecule is demonstrated in FIG. 34 employing a GITR Luciferase assay. Activity of the trivalent GITR agonist aPDL1-scGITRL is clearly enhanced by cross-linking with anti-human Fc (x-link) to the level observed for the hexavalent GITR agonist scGITRL-Fc.

The application is further characterized by its claims and items 1-11 below.

Item 1: A multispecific TNF family fusion protein assembly comprising at least,
   (a) a single-chain TNF-SF receptor binding domain superfamily ligand fused to
   (b) a first peptide linker fused to
   (c) a first hetero-dimerization domain and
   (d) an antigen binding or interacting protein moiety fused to
   (e) a second peptide linker fused to
   (f) a second hetero-dimerization domain
Item 2: A multispecific TNF family fusion protein assembly comprising at least
   (a) a single-chain TNF-SF receptor binding domain fused to
   (b) a first peptide linker fused to
   (c) a first hetero-dimerization domain and a
   (d) and a second single-chain TNF-SF receptor binding domain fused to
   (e) a second peptide linker fused to
   (f) a second hetero-dimerization domain
Item 3: A multispecific TNF family fusion protein assembly comprising at least
   (a) a functional Fab domain of an antibody fused to
   (b) a single-chain TNF-SF receptor binding domain, wherein the c-terminal end of the constant heavy chain domain of the Fab fragment (a) is fused to the single-chain TNF-SF receptor binding via a peptide linker.
Item 4: A multispecific TNF family fusion protein assembly comprising
   (a) assembly consisting of SEQ-ID33 and SEQ-ID 46 and SEQ-ID: 47,
   (b) assembly consisting of SEQ-ID37 and SEQ-ID 46 and SEQ-ID: 47,
   (c) assembly consisting of SEQ-ID41 and SEQ-ID 46 and SEQ-ID: 47,
Item 5: A multispecific TNF family fusion protein assembly selected from the list comprising
   (a) assembly consisting of SEQ-ID33 and SEQ-ID 54 and SEQ-ID: 55,
   (b) assembly consisting of SEQ-ID37 and SEQ-ID 54 and SEQ-ID: 55
   (c) assembly consisting of SEQ-ID41 and SEQ-ID 54 and SEQ-ID: 55
Item 6: A multispecific TNF family fusion protein assembly selected from the list comprising
   (a) assembly consisting of SEQ-ID33 and SEQ-ID 50 and SEQ-ID: 51,
   (b) assembly consisting of SEQ-ID37 and SEQ-ID 50 and SEQ-ID: 51
   (c) assembly consisting of SEQ-ID41 and SEQ-ID 50 and SEQ-ID: 51
Item 7: A nucleic acid molecule encoding the protein moiety of Item 1 part a) - c) and a nucleic acid encoding the protein moiety of Item 1 part d) - e).
Item 8. A nucleic acid molecule encoding the protein moiety of Item 2 part a) - c) and a nucleic acid encoding the protein moiety of Item 2 part d) - e).
Item 9: A nucleic acid molecule encoding a protein of any one of Items 3 or nucleic acid molecules for coexpression of protein assemblies of any one of Items 4-6
Item 10: A host cell comprising a nucleic acids of any one of Items 7-9.
Item 11: A pharmaceutical composition comprising at least a multispecific protein assembly of any one of claims 1-6 or a nucleic acid of 7-9.

1-42. (canceled)

43. A bispecific TNF superfamily fusion protein assembly comprising at least
(a) a single-chain TNF-SF receptor binding domain fused to, wherein the single-chain TNF-SF receptor binding domain is a single-chain CD27 receptor binding domain of SEQ ID NO:70 or SEQ ID NO: 119,
(b) a first peptide linker which is a hinge-linker according to SEQ ID NO: 12 fused to
(c) a first (hetero-)dimerization domain which is an IgG1 based Fc-domain, preferably of SEQ ID NO:20, SEQ ID NO:22 or SEQ ID NO:24, and
(d) an antigen binding or interacting protein moiety fused to
(e) a second peptide linker fused to
(f) a second (hetero-)dimerization domain.

44. The bispecific TNF superfamily fusion protein assembly of claim 43, wherein hetero-dimerization of dimerization domains c) and f) is enforced by CH3-domains, wherein the second (hetero-)dimerization domain (f) is independently selected from IgG1 or IgG2 or IgG3 or IgG4 or IgA or IgD derived CH3-domains, and which are additionally stabilized by hinge interchain cysteines.

45. The bispecific TNF superfamily fusion protein assembly of claim 43, wherein the antigen binding or interacting protein moiety d) is an IgG antibody-derived heavy and light chain, or an antibody fragment selected from the group consisting of Fv, Fab, Fab′, Fab′-SH, F(ab′)2; diabodies, triabodies, tetrabodies, cross-Fab fragments; or a single-chain antibody (e.g. scFv) or a single domain antibody.

46. The bispecific TNF superfamily fusion protein assembly of claim 43, wherein the antigen binding or interacting protein moiety d) is an IgG antibody-derived heavy and light chain with specificity selected from the group consisting of anti-PD-L1, anti-CD137, anti-Mesothelin, anti-CD25, anti-PD-1, anti-CEA or anti-CD95L.

47. The bispecific TNF superfamily fusion protein assembly of claim 43, wherein the antigen binding or interacting protein moiety d) is an IgG antibody-derived heavy and light chain that has the specificity selected from the group consisting of anti-PD-L1(SEQ-ID:74 aPD-L1-VH, SEQ-ID:75 aPD-L1-VL), or anti-CD137 (SEQ-ID:59 aCD137-VH, SEQ-ID:60 aCD137-VL), or anti-Mesothelin (SEQ-ID:61 aMeso-VH, SEQ-ID:62 aMeso-VL), or anti-CD25 (SEQ-ID:63 aCD25-VH, SEQ-ID:64 aCD25-VL), or anti-PD-1 (SEQ-ID:65 aPD1-a-VH, SEQ-ID:66 aPD1-a-VL, SEQ-ID:67 aPD1-b-VH, SEQ-ID:68 aPD1-b-VL), or anti-CEA (SEQ-ID:113 aCEA-a-VH, SEQ-ID:114 aCEA-a-VL, SEQ-ID:115 aCEA-b-VH, SEQ-ID:116 aCEA-b-VL; SEQ-ID:117 aCD95L-VH, SEQ-ID:118 aCD95L-VL.

48. The bispecific TNF superfamily fusion protein assembly of claim 43, wherein the trivalent single-chain TNFSF-RBD-Fc is a scCD27L-Fc of SEQ-ID:37 (scCD27L-Fc-knob_b) and wherein the IgG derived antibody of d) comprises SEQ-ID:55 (aPD-L1-LC) and/or SEQ-ID:54 (aPD-L1-HC-RF-hole).

49. The bispecific TNF superfamily fusion protein assembly of claim 43, wherein the trivalent single-chain TNFSF-RBD-Fc is a scCD27L-Fc of SEQ-ID:37 (scCD27L-Fc-knob_b) and wherein the IgG derived antibody of d) comprises SEQ-ID:47 (aCD95L-LC) and/or SEQ-ID:46 (aCD95L-HC-RF-hole).

50. The bispecific TNF superfamily fusion protein assembly of claim 43, wherein the trivalent single-chain TNFSF-RBD-Fc is a scCD27L-Fc of SEQ-ID:37 (scCD27L-Fc-knob_b) and wherein the IgG derived antibody of d) comprises SEQ-ID:51 (aCEA-LC) and/or SEQ-ID:50 (aCEA-HC-RF-hole).

51. The bispecific TNF superfamily fusion protein assembly of claim 43, wherein the dimerizing CH3 domain (f) is selected from CH3 domains represented as part of SEQ-ID:44, SEQ-ID:45, SEQ-ID:48, SEQ-ID:49, SEQ-ID:52, SEQ-ID:53, SEQ-ID:28, SEQ-ID:29, SEQ-ID:30, SEQ-ID:31, SEQ-ID:111, SEQ-ID:112, SEQ-ID:20, SEQ-ID:21, SEQ-ID:22, SEQ-ID:23, SEQ-ID:24, SEQ-ID:25, SEQ-ID: 109, or SEQ-ID:110.

52. A nucleic acid molecule encoding the part a) - c) protein moieties of claim 43 and/or a nucleic acid molecule encoding the part d) - e) protein moieties of claim 43.

53. The nucleic acid molecule of claim 52 for expression or coexpression of proteins or protein assemblies.

54. A vector comprising at least one nucleic acid molecule according to claim 52.

55. A host cell comprising the nucleic acid of claim 52.

56. A pharmaceutical composition comprising the protein assembly of claim 43.
